SEQUENCE LISTING (50 sequences)

SEQ ID NO: 1 - protein, 277 aa, Pavlova sp. CCMP459
MMLAAGYLLVLSAARQSFQQDIDNPNGAYSTSWTGLPIVMSVVYLSGVFGLTKYFENRKPMTGLKDYMFTYNLYQVIINVWCVVAFLLEVRRAGMSLIGNKVDLGPNSFRLGFVTWVHYNNKYVELLDTLWMVLRKKTQQVSFLHVYHHVLLMWAWFVVVKLGNGGDAYFGGLMNSIIHVMMYSYYTMALLGWSCPWKRYLTQAQLVQFCICLAHSTWAAVTGAYPWRICLVEVWVMVSMLVLFTRFYRQAYAKEAKAKEAKKLAQEASQAKAVKAE

SEQ ID NO: 2 - DNA, 897 bp, Euglena gracilis
atggcggatagcccagtcatcaacctcagcaccatgtggaaacccctttcactgatgggacatgtctggaagcaggcacaacaggagggcagcatttcggcctatgctgattctgttcggattcctctcattatgtccgttttatacttatcaatgatcttcgtggggtgccgctggatgaagaaccgtgagccctttgagatcaaaacatacatgtttgcgtataacctgtatcagaccttgatgaacctttgcatcgtgttgggattcttgtaccaggtgcatgccactgggatgcgcttttggggaagtggtgtcgaccgaagcccgaaaggtttgggcattggcttcttcatttatgcccactaccacaacaagtatgtggaatattttgatacactttttatggtgctgcgaaagaagaacaaccagatttctttccttcacgtgtatcatcatgccctgttgacatgggcttggtttgctgttgtgtatttcgcacctggaggtgatggctggtttggagcttgctacaattcttccatccatgtcctgatgtactcttactacttgcttgcaacttttggcatcagttgcccatggaagaagatcttgacacagctccagatggttcagttctgtttctgttttacacattccatttatgtgtggatttgcgggtcagagatctacccacggcctctgactgctttgcagtcgttcgtgatggtcaatatgttggtgctgtttggcaatttctatgtcaagcaatactcccaaaagaacggcaagccggagaacggagccacccctgagaacggagcgaagccgcaaccttgcgagaacggcacggtggaaaagcgagaggcgccccgatctgtcggcatgggacgctga

SEQ ID NO: 3 - protein, 298 aa, Euglena gracilis
MADSPVINLSTMWKPLSLMGHVWKQAQQEGSISAYADSVRIPLIMSVLYLSMIFVGCRWMKNREPFEIKTYMFAYNLYQTLMNLCIVLGFLYQVHATGMRFWGSGVDRSPKGLGIGFFIYAHYHNKYVEYFDTLFMVLRKKNNQISFLHVYHHALLTWAWFAVVYFAPGGDGWFGACYNSSIHVLMYSYYLLATFGISCPWKKILTQLQMVQFCFCFTHSIYVWICGSEIYPRPLTALQSFVMVNMLVLFGNFYVKQYSQKNGKPENGATPENGAKPQPCENGTVEKREAPRSVGMGR

SEQ ID NO: 4 - DNA, 2382 bp, Euglena gracilis
atggcggatagcccagtcatcaacctcagcaccatgtggaaacccctttcactgatggctttggaccttgccgttttgggacatgtctggaagcaggcacaacaggagggcagcatttcggcctatgctgattctgtttggactcctctcattatgtccggtttatacttatcaatgatcttcgtggggtgccgctggatgaagaaccgtgaaccctttgagatcaaaacatacatgtttgcgtataacctgtatcagaccttgatgaacctttgcatcgtgttgggattcttgtaccaggtgcatgccactgggatgcgcttttggggaagtggtgtcgaccgaagcccaaaaggtttgggcattggcttcttcatttatgcccactaccacaacaagtatgtggaatattttgatacactttttatggtgctgcgaaagaagaacaaccagatttctttccttcacgtgtatcatcatgccctgttgacatgggcttggtttgctgttgtgtatttcgcacctggaggtgatggctggtttggagcttgctacaattcttccatccatgtcctgatgtactcttactacttgcttgcaacttttggcatcagttgcccatggaagaagatcttgacacagctccagatggttcaattctgtttctgttttacacattccatttatgtgtggatttgcgggtcagagatctacccacggcctctgactgctttgcagtcgttcgtgatggtcaatatgttggtgctgtttggcaatttctatgtcaagcaatactcccaaaagaacggcaagccggagaacggagccacccctgagaacggagcgaagccgcaaccttgcgagaacggcacggtggaaaagcgagagaatgacaccgccaacgttcggcccgcccgtccagctggactcccgccggccacgtactacgactccctggcagtgtcggggcagggcaaggagcggctgttcaccaccgatgaggtgaggcggcacatcctccccaccgatggctggctgacgtgccacgaaggagtctacgatgtcactgatttccttgccaagcaccctggtggcggtgtcatcacgctgggccttggaagggactgcacaatcctcgtcgagtcataccaccctgctgggcgcccggacaaggtgatggagaagtaccgcattggtacgctgcaggaccccaagacgttctatgcttggggagagtccgatttctaccctgagttgaagcgccgggcccttgcaaggctgaaggaggctggtcaggcgcggcgcggcggccttggggtgaaggccctcctggtgctcaccctcttcttcgtgtcgtggtacatgtgggtggcccacaagtccttcctctgggccgccgtctggggcttcgccggctcccacgtcgggctgagcatccagcacgacggcaaccacggcgcgttcagccgcagcacactggtgaaccgcctggcggggtggggcatggacttgatcggcgcgtcgtcaacggtgtgggagtaccagcacgtcatcggccaccaccagtacaccaacctcgtgtcggacacgctattcagtctgcctgagaacgatccggacgtcttctccagctacccgctgatgcgcatgcacccggatacggcgtggcagccgcaccaccgcttccagcacctgttcgcgttcccactgttcgccctgatgacaatcagcaaggtgctgaccagcgatttcgctgtctgcctcagcatgaagaaggggtccatcgactgctcctccaggctcgtcccactggaggggcagctgctgttctggggggccaagctggcgaacttcctgttgcagattgtgttgccatgctacctccacgggacagctatgggcctggccctcttctctgttgcccaccttgtgtcgggggagtacctcgcgatctgcttcatcatcaaccacatcagcgagtcttgtgagtttatgaatacaagctttcaaaccgccgcccggaggacagagatgcttcaggcagcccatcaggcagcggaggccaagaaggtgaagcccacccctccaccgaacgattgggctgtgacacaggtccaatgctgcgtgaattggagatcaggtggcgtgttggccaatcacctctctggaggcttgaaccaccagatcgagcatcatctgttccccagcatctcgcatgccaactaccccatcatcgcccgtgttgtgaaggaggtgtgcgaggagtatgggttgccgtacaagaactacgtcacgttctgggatgcagtctgtggcatggttcagcacctccggttgatgggtgctccaccggtgccaacgaacggggacaaaaagtcataa

SEQ ID NO: 5 - protein, 793 aa, Euglena gracilis
MADSPVINLSTMWKPLSLMALDLAVLGHVWKQAQQEGSISAYADSVWTPLIMSGLYLSMIFVGCRWMKNREPFEIKTYMFAYNLYQTLMNLCIVLGFLYQVHATGMRFWGSGVDRSPKGLGIGFFIYAHYHNKYVEYFDTLFMVLRKKNNQISFLHVYHHALLTWAWFAVVYFAPGGDGWFGACYNSSIHVLMYSYYLLATFGISCPWKKILTQLQMVQFCFCFTHSIYVWICGSEIYPRPLTALQSFVMVNMLVLFGNFYVKQYSQKNGKPENGATPENGAKPQPCENGTVEKRENDTANVRPARPAGLPPATYYDSLAVSGQGKERLFTTDEVRRHILPTDGWLTCHEGVYDVTDFLAKHPGGGVITLGLGRDCTILVESYHPAGRPDKVMEKYRIGTLQDPKTFYAWGESDFYPELKRRALARLKEAGQARRGGLGVKALLVLTLFFVSWYMWVAHKSFLWAAVWGFAGSHVGLSIQHDGNHGAFSRSTLVNRLAGWGMDLIGASSTVWEYQHVIGHHQYTNLVSDTLFSLPENDPDVFSSYPLMRMHPDTAWQPHHRFQHLFAFPLFALMTISKVLTSDFAVCLSMKKGSIDCSSRLVPLEGQLLFWGAKLANFLLQIVLPCYLHGTAMGLALFSVAHLVSGEYLAICFIINHISESCEFMNTSFQTAARRTEMLQAAHQAAEAKKVKPTPPPNDWAVTQVQCCVNWRSGGVLANHLSGGLNHQIEHHLFPSISHANYPIIARVVKEVCEEYGLPYKNYVTFWDAVCGMVQHLRLMGAPPVPTNGDKKS

SEQ ID NO: 6 - protein, 541 aa, Euglena gracilis
  MISC_FEATURE (1)..(541): NCBI Accession No. AAQ19605 (GI 33466346), locus AAQ19605, CDS AY278558
MLVLFGNFYVKQYSQKNGKPENGATPENGAKPQPCENGTVEKRENDTANVRPTRPAGPPPATYYDSLAVSGQGKERLFTTDEVRRHILPTDGWLTCHEGVYDVTDFLAKHPGGGVITLGLGRDCTILIESYHPAGRPDKVMEKYRIGTLQDPKTFYAWGESDFYPELKRRALARLKEAGQARRGGLGVKALLVLTLFFVSWYMWVAHKSFLWAAVWGFAGSHVGLSIQHDGNHGAFSRNTLVNRLAGWGMDLIGASSTVWEYQHVIGHHQYTNLVSDTLFSLPENDPDVFSSYPLMRMHPDTAWQPHHRFQHLFAFPLFALMTISKVLTSDFAVCLSMKKGSIDCSSRLVPLEGQLLFWGAKLANFLLQIVLPCYLHGTAMGLALFSVAHLVSGEYLAICFIINHISESCEFMNTSFQTAARRTEMLQAAHQAAEAKKVKPTPPPNDWAVTQVQCCVNWRSGGVLANHLSGGLNHQIEHHLFPSISHANYPTIAPVVKEVCEEYGLPYKNYVTFWDAVCGMVQHLRLMGAPPVPTNGDKKS

SEQ ID NO: 7 - DNA, 2382 bp, Euglena gracilis
atggcggatagcccagtcatcaacctcagcaccatgtggaaacccctttcactgatggctttggaccttgccattttgggacatgtctggaagcaggcacaacaggagggcagcatttcggcctatgctgattctgtttggactcctctcattatgtccgttttatacttatcaatgatcttcgtggggtgccgctggatgaagaaccgtgaaccctttgagatcaaaacatacatgtttgcgtataacctgtatcagaccttgatgaacctttgcatcgtgttgggattcttgtaccaggtgcatgccactgggatgcgcttttggggaagtggtgtcgaccgaagcccgaaaggtttgggcattggcttcttcatttatgcccactaccacaacaagtatgtggaatattttgatacactttttatggtgctgcgaaagaagaacaaccagatttctttccttcacgtgtatcatcatgccctgttgacatgggcttggtttgctgttgtgtatttcgcacctggaggtgatggctggtttggagcttgctacaattcttccatccatgtcctgatgtactcttactacttgcttgcaacttttggcatcagttgcccatggaagaagatcttgacacagctccagatggttcaattctgtttctgttttacacattccatttatgtgtggatttgcgggtcagagatctacccacggcctctgactgctttgcagtcgttcgtgatggtcaatatgttggtgctgtttggcaatttctatgtcaagcaatactcccaaaagaacggcaagccggagaacggagccacccctgagaacggagcgaagccgcaaccttgcgagaacggcacggtggaaaagcgagagaatgacaccgccaacgttcggcccacccgtccagctggacccccgccggccacgtactacgactccctggcagtgtcggggcagggcaaggagcggctgttcaccaccgatgaggtgaggcggcacatcctccccaccgatggctggctgacgtgccacgaaggagtctacgatgtcactgatttccttgccaagcaccctggtggcggtgtcatcacgctgggccttggaagggactgcacaatcctcatcgagtcataccaccctgctgggcgcccggacaaggtgatggagaagtaccgcattggtacgctgcaggaccccaagacgttctatgcttggggagagtccgatttctaccctgagttgaagcgccgggcccttgcaaggctgaaggaggctggtcaggcgcggcgcggcggccttggggtgaaggccctcctggtgctcaccctcttcttcgtgtcgtggtacatgtgggtggcccacaagtccttcctctgggccgccgtctggggcttcgccggctcccacgtcgggctgagcatccagcacgatggcaaccacggcgcgttcagccgcaacacactggtgaaccgcctggcggggtggggcatggacttgatcggcgcgtcgtccacggtgtgggagtaccagcacgtcatcggccaccaccagtacaccaacctcgtgtcggacacgctattcagtctgcctgagaacgatccggacgtcttctccagctacccgctgatgcgcatgcacccggatacggcgtggcagccgcaccaccgcttccagcacctgttcgcgttcccactgttcgccctgatgacaatcagcaaggtgctgaccagcgatttcgctgtctgcctcagcatgaagaaggggtccatcgactgctcctccaggctcgtcccactggaggggcagctgctgttctggggggccaagctggcgaacttcctgttgcagattgtgttgccatgctacctccacgggacagctatgggcctggccctcttctctgttgctcaccttgtgtcgggggagtacctcgcgatctgcttcatcatcaaccacatcagcgagtcttgtgagtttatgaatacaagctttcaaaccgccgcccggaggacagagatgcttcaggcagcacatcaggcagcggaggccaagaaggtgaagcccacccctccaccgaacgattgggctgtgacacaggtccaatgctgcgtgaattggagatcaggtggcgtgttggccaatcacctctctggaggcttgaaccaccagatcgagcatcatctgttccccagcatctcgcatgccaactaccccaccatcgcccctgttgtgaaggaggtgtgcgaggagtacgggttgccgtacaagaattacgtcacgttctgggatgcagtctgtggcatggttcagcacctccggttgatgggtgctccaccggtgccaacgaacggggacaaaaagtcataa

SEQ ID NO: 8 - protein, 793 aa, Euglena gracilis
MADSPVINLSTMWKPLSLMALDLAILGHVWKQAQQEGSISAYADSVWTPLIMSVLYLSMIFVGCRWMKNREPFEIKTYMFAYNLYQTLMNLCIVLGFLYQVHATGMRFWGSGVDRSPKGLGIGFFIYAHYHNKYVEYFDTLFMVLRKKNNQISFLHVYHHALLTWAWFAVVYFAPGGDGWFGACYNSSIHVLMYSYYLLATFGISCPWKKILTQLQMVQFCFCFTHSIYVWICGSEIYPRPLTALQSFVMVNMLVLFGNFYVKQYSQKNGKPENGATPENGAKPQPCENGTVEKRENDTANVRPTRPAGPPPATYYDSLAVSGQGKERLFTTDEVRRHILPTDGWLTCHEGVYDVTDFLAKHPGGGVITLGLGRDCTILIESYHPAGRPDKVMEKYRIGTLQDPKTFYAWGESDFYPELKRRALARLKEAGQARRGGLGVKALLVLTLFFVSWYMWVAHKSFLWAAVWGFAGSHVGLSIQHDGNHGAFSRNTLVNRLAGWGMDLIGASSTVWEYQHVIGHHQYTNLVSDTLFSLPENDPDVFSSYPLMRMHPDTAWQPHHRFQHLFAFPLFALMTISKVLTSDFAVCLSMKKGSIDCSSRLVPLEGQLLFWGAKLANFLLQIVLPCYLHGTAMGLALFSVAHLVSGEYLAICFIINHISESCEFMNTSFQTAARRTEMLQAAHQAAEAKKVKPTPPPNDWAVTQVQCCVNWRSGGVLANHLSGGLNHQIEHHLFPSISHANYPTIAPVVKEVCEEYGLPYKNYVTFWDAVCGMVQHLRLMGAPPVPTNGDKKS

SEQ ID NO: 9 - DNA, 2569 bp, Euglena gracilis
gcggccgctagacatgccaccagttgtggtatggttaaccgaatatgaacaaacacccgattccagtccaagattgctcaataaggaacagggtgggggagcaacttgtgagagtttcgacgccatgggtgcctacagattaagtagatccttcttgtccaacttgttgaccttgcgctcggcacattctgagagctggtttcgcagccggtagatttcgtcgttggcctgttgcaactgggtccgcagctcggagtcgcaaagattgttcgctgtgaggatctcagcacggatttcttctaattcagaagccgcagcacggagttgattcccaagggtatcaaggcggccatatgcttgctccaataatttctcaacacggtcgggggtgatgctcagcttaacagattcttctggtggttttggaagctctccagcaggctccacggggggcgcagggggcgcggcgggaaccagtggaagagttggcaacgtcgacagggggtactgtgcttgagggccaggaatcacggacgggacttgcggaatgtaagatggaccagtaggaaatgacggaaatgttcgagcaaatccgccaaaacccgtgtacgcaggtgccggggcgtaattaacaatcgacagcggccgcgaattcgcggccgctcacattccatttatgtgtggatttgcgggtcagagatctacccacggcctctgactgctttgcagtcgttcgtgatggtcaatatgttggtgctgtttggcaatttctatgtcaagcaatactcccaaaagaacggcaagccggagaacggagccacccctgagaacggagcgaagccgcaaccttgcgagaacggcacggtggaaaagcgagagaatgacaccgccaacgttcggcccacccgtccagctggacccccgccggccacgtactacgactccctggcagtgtcggggcagggcaaggagcggctgttcaccaccgatgaggtgaggcggcacatcctccccaccgatggctggctgacgtgccacgaaggagtctacgatgtcactgatttccttgccaagcaccctggtggcggtgtcatcacgctgggccttggaagggactgcacaatcctcatcgagtcataccaccctgctgggcgcccggacaaggtgatggagaagtaccgcattggtacgctgcaggaccccaagacgttctatgcttggggagagtccgatttctaccctgagttgaagcgccgggcccttgcaaggctgaaggaggctggtcaggcgcggcgcggcggccttggggtgaaggccctcctggtgctcaccctcttcttcgtgtcgtggtacatgtgggtggcccacaagtccttcctctgggccgccgtctggggcttcgccggctcccacgtcgggctgagcatccagcacgatggcaaccacggcgcgttcagccgcaacacactggtgaaccgcctggcggggtggggcatggacttgatcggcgcgtcgtccacggtgtgggagtaccagcacgtcatcggccaccaccagtacaccaacctcgtgtcggacacgctattcagtctgcctgagaacgatccggacgtcttctccagctacccgctgatgcgcatgcacccggatacggcgtggcagccgcaccaccgcttccagcacctgttcgcgttcccactgttcgccctgatgacaatcagcaaggtgctgaccagcgatttcgctgtctgcctcagcatgaagaaggggtccatcgactgctcctccaggctcgtcccactggaggggcagctgctgttctggggggccaagctggcgaacttcctgttgcagattgtgttgccatgctacctccacgggacagctatgggcctggccctcttctctgttgctcaccttgtgtcgggggagtacctcgcgatctgcttcatcatcaaccacatcagcgagtcttgtgagtttatgaatacaagctttcaaaccgccgcccggaggacagagatgcttcaggcagcacatcaggcagcggaggccaagaaggtgaagcccacccctccaccgaacgattgggctgtgacacaggtccaatgctgcgtgaattggagatcaggtggcgtgttggccaatcacctctctggaggcttgaaccaccagatcgagcatcatctgttccccagcatctcgcatgccaactaccccaccatcgcccctgttgtgaaggaggtgtgcgaggagtacgggttgccgtacaagaattacgtcacgttctgggatgcagtctgtggcatggttcagcacctccggttgatgggtgctccaccggtgccaacgaacggggacaaaaagtcataagccacgacatcatttggggctcactccgtgcagccttttcttgggctgcccacgaagatgcgcgatgaggcacctggtggttgccctccgccggcctcggaaaacggttcgacgcctgctccttcagcccagagcactccggcgaagagtgaaagagcactgacctgaattttatgatgacccatttagcggccgc

SEQ ID NO: 10 - DNA, 1626 bp, Euglena gracilis
atgttggtgctgtttggcaatttctatgtcaagcaatactcccaaaagaacggcaagccggagaacggagccacccctgagaacggagcgaagccgcaaccttgcgagaacggcacggtggaaaagcgagagaatgacaccgccaacgttcggcccacccgtccagctggacccccgccggccacgtactacgactccctggcagtgtcggggcagggcaaggagcggctgttcaccaccgatgaggtgaggcggcacatcctccccaccgatggctggctgacgtgccacgaaggagtctacgatgtcactgatttccttgccaagcaccctggtggcggtgtcatcacgctgggccttggaagggactgcacaatcctcatcgagtcataccaccctgctgggcgcccggacaaggtgatggagaagtaccgcattggtacgctgcaggaccccaagacgttctatgcttggggagagtccgatttctaccctgagttgaagcgccgggcccttgcaaggctgaaggaggctggtcaggcgcggcgcggcggccttggggtgaaggccctcctggtgctcaccctcttcttcgtgtcgtggtacatgtgggtggcccacaagtccttcctctgggccgccgtctggggcttcgccggctcccacgtcgggctgagcatccagcacgatggcaaccacggcgcgttcagccgcaacacactggtgaaccgcctggcggggtggggcatggacttgatcggcgcgtcgtccacggtgtgggagtaccagcacgtcatcggccaccaccagtacaccaacctcgtgtcggacacgctattcagtctgcctgagaacgatccggacgtcttctccagctacccgctgatgcgcatgcacccggatacggcgtggcagccgcaccaccgcttccagcacctgttcgcgttcccactgttcgccctgatgacaatcagcaaggtgctgaccagcgatttcgctgtctgcctcagcatgaagaaggggtccatcgactgctcctccaggctcgtcccactggaggggcagctgctgttctggggggccaagctggcgaacttcctgttgcagattgtgttgccatgctacctccacgggacagctatgggcctggccctcttctctgttgctcaccttgtgtcgggggagtacctcgcgatctgcttcatcatcaaccacatcagcgagtcttgtgagtttatgaatacaagctttcaaaccgccgcccggaggacagagatgcttcaggcagcacatcaggcagcggaggccaagaaggtgaagcccacccctccaccgaacgattgggctgtgacacaggtccaatgctgcgtgaattggagatcaggtggcgtgttggccaatcacctctctggaggcttgaaccaccagatcgagcatcatctgttccccagcatctcgcatgccaactaccccaccatcgcccctgttgtgaaggaggtgtgcgaggagtacgggttgccgtacaagaattacgtcacgttctgggatgcagtctgtggcatggttcagcacctccggttgatgggtgctccaccggtgccaacgaacggggacaaaaagtcataa

SEQ ID NO: 11 - protein, 300 aa, Ostreococcus tauri
MSASGALLPAIASAAYAYATYAYAFEWSHANGIDNVDAREWIGALSLRLPAIATTMYLLFCLVGPRLMAKREAFDPKGFMLAYNAYQTAFNVVVLGMFAREISGLGQPVWGSTMPWSDRKSFKILLGVWLHYNNKYLELLDTVFMVARKKTKQLSFLHVYHHALLIWAWWLVCHLMATNDCIDAYFGAACNSFIHIVMYSYYLMSALGIRCPWKRYITQAQMLQFVIVFAHAVFVLRQKHCPVTLPWAQMFVMTNMLVLFGNFYLKAYSNKSRGDGASSVKPAETTRAPSVRRTRSRKID

SEQ ID NO: 12 - protein, 358 aa, Thalassiosira pseudonana
MCSSPPSQSKTTSLLARYTTAALLLLTLTTWCHFAFPAATATPGLTAEMHSYKVPLGLTVFYLLSLPSLKYVTDNYLAKKYDMKSLLTESMVLYNVAQVLLNGWTVYAIVDAVMNRDHPFIGSRSLVGAALHSGSSYAVWVHYCDKYLEFFDTYFMVLRGKMDQVSFLHIYHHTTIAWAWWIALRFSPGGDIYFGALLNSIIHVLMYSYYALALLKVSCPWKRYLTQAQLLQFTSVVVYTGCTGYTHYYHTKHGADETQPSLGTYYFCCGVQVFEMVSLFVLFSIFYKRSYSKKNKSGGKDSKKNDDGNNEDQCHKAMKDISEGAKEVVGHAAKDAGKLVATASKAVKRKGTRVTGAM

SEQ ID NO: 13 - protein, 515 aa, Thraustochytrium aureum
  MISC_FEATURE (1)..(515): NCBI Accession No. AAN75707(GI 25956288), locus AAN75707, CDS AF391543
MTVGFDETVTMDTVRNHNMPDDAWCAIHGTVYDITKFSKVHPGGDIIMLAAGKEATILFETYHIKGVPDAVLRKYKVGKLPQGKKGETSHMPTGLDSASYYSWDSEFYRVLRERVAKKLAEPGLMQRARMELWAKAIFLLAGFWGSLYAMCVLDPHGGAMVAAVTLGVFAAFVGTCIQHDGSHGAFSKSRFMNKAAGWTLDMIGASAMTWEMQHVLGHHPYTNLIEMENGLAKVKGADVDPKKVDQESDPDVFSTYPMLRLHPWHRQRFYHKFQHLYAPLIFGFMTINKVISQDVGVVLRKRLFQIDANCRYGSPWNVARFWIMKLLTTLYMVALPMYMQGPAQGLKLFFMAHFTCGEVLATMFIVNHIIEGVSYASKDAVKGVMAPPRTVHGVTPMQVTQKALSAAESTKSDADKTTMIPLNDWAAVQCQTSVNWAVGSWFWNHFSGGLNHQIEHHCFPQNPHTVNVYISGIVKETCEEYGVPYQAEISLFSAYFKMLSHLRTLGNEDLTAWST

SEQ ID NO: 14 - protein, 509 aa, Schizochytrium aggregatum
MTVGGDEVYSMAQVRDHNTPDDAWCAIHGEVYELTKFARTHPGGDIILLAAGKEATILFETYHVRPISDAVLRKYRIGKLAAAGKDEPANDSTYYSWDSDFYKVLRQRVVARLEERKIARRGGPEIWIKAAILVSGFWSMLYLMCTLDPNRGAILAAIALGIVAAFVGTCIQHDGNHGAFAFSPFMNKLSGWTLDMIGASAMTWEMQHVLGHHPYTNLIEMENGTQKVTHADVDPKKADQESDPDVFSTYPMLRLHPWHRKRFYHRFQHLYAPLLFGFMTINKVITQDVGVVLSKRLFQIDANCRYASKSYVARFWIMKLLTVLYMVALPVYTQGLVDGLKLFFIAHFSCGELLATMFIVNHIIEGVSYASKDSVKGTMAPPRTVHGVTPMHDTRDALGKEKAATKHVPLNDWAAVQCQTSVNWSIGSWFWNHFSGGLNHQIEHHLFPGLTHTTYVYIQDVVQATCAEYGVPYQSEQSLFSAYFKMLSHLRALGNEPMPSWEKDHPKSK

SEQ ID NO: 15 - protein, 550 aa, Thalassiosira pseudonana
  MISC_FEATURE (1)..(550): NCBI Accession No. AAX14506 (GI 60173017), locus AAX14506, CDS AY817156
MGNGNLPASTAQLKSTSKPQQQHEHRTISKSELAQHNTPKSAWCAVHSTPATDPSHSNNKQHAHLVLDITDFASRHPGGDLILLASGKDASVLFETYHPRGVPTSLIQKLQIGVMEEEAFRDSFYSWTDSDFYTVLKRRVVERLEERGLDRRGSKEIWIKALFLLVGFWYCLYKMYTTSDIDQYGIAIAYSIGMGTFAAFIGTCIQHDGNHGAFAQNKLLNKLAGWTLDMIGASAFTWELQHMLGHHPYTNVLDGVEEERKERGEDVALEEKDQESDPDVFSSFPLMRMHPHHTTSWYHKYQHLYAPPLFALMTLAKVFQQDFEVATSGRLYHIDANVRYGSVWNVMRFWAMKVITMGYMMGLPIYFHGVLRGVGLFVIGHLACGELLATMFIVNHVIEGVSYGTKDLVGGASHGDEKKIVKPTTVLGDTPMEKTREEALKSNSNNNKKKGEKNSVPSVPFNDWAAVQCQTSVNWSPGSWFWNHFSGGLSHQIEHHLFPSICHTNYCHIQDVVESTCAEYGVPYQSESNLFVAYGKMISHLKFLGKAKCE

SEQ ID NO: 16 - protein, 433 aa, Isochrysis galbana
  MISC_FEATURE (1)..(433): NCBI Accession No. AAV33631 (GI 54307110), locus AAV33631, CDS AY630574
MCNAAQVETQALRAKEAAKPTWTKIHGRTVDVETFRHPGGNILDLFLGMDATTAFETFHGHHKGAWKMLKTLPEKEVAAADIPAQKEEHVAEMTRLMASWRERGLFKPRPVASSIYGLCVIFAIAASVACAPYAPVLAGIAVGTCWAQCGFLQHMGGHREWGRTWSFAFQHLFEGLLKGGSASWWRNRHNKHHAKTNVLGEDGDLRTTPFFAWDPTLAKKVPDWSLRTQAFTFLPALGAYVFVFAFTVRKYSVVKRLWHEVALMVAHYALFSWALSAAGASLSSGLTFYCTGYAWQGIYLGFFFGLSHFAVERVPSTATWLESTMMGTVDWGGSSAFCGYLSGFLNIQIEHHMAPQMPMENLRQIRADCKAAAHKFGLPYRELTFVAATKLMMSGLYRTGKDELKLRADRRKFTRAQAYMGAASALVDTLKAD

SEQ ID NO: 17 - DNA, 2523 bp, Euglena anabaena
atggccgaaggcaagagcgatgggcctgtggtgacccttcaaagcatgtggaaaccgcttgctttgatggcagtagatgtcggcatattggtcaatgtccgccgcaaggctttcactgagtttgatgggcacagcaacgtttttgcagatccagtttacattccatttgtgatgaatctcttctacttgaccatgatctttgctgggtgccgttggatgaagactcgcgagccctttgagatcaagtcatatatgtttgcatacaatgcatatcagacaatgatgaacttcctcattgtcgtcgggttcatgtatgaggtgcacagcacagggatgcgatattgggggtccaggatcgacacctccaccaagggcttgggcctcggtttcctgatctatgcccactaccacaacaaatatgtggagtatgtcgacacgttgttcatgatcctgcgcaagaaaaacaaccagatctcgttcctccacgtctaccaccattcgcttttgacttgggcctggtgggccgtggtctactgggccccgggaggagatgcctggttcggagcatgctacaattccttcatccacgtgctgatgtactcctactacctgtttgcaacgtttggcatcaggtgcccctggaagaagatgctgacccagttgcagatggtgcaattctgcttctgctttgcccacgcgatgtatgttggatggctgggccatgaagtctacccgcgctggttgacggcgctgcaggcatttgtgatgctaaacatgctggtgctgttcggcaacttctacatgaagtcgtactccaaggccagcaagctggagccggcctcccccgtgtcccccgcctccctcgcccagaaaccgttcgagaacgccaaggtgaagcctgggggccccggcaagccaagcgagattgcgtcgctgccaccgccaattcgaccagtcgggaacccacctgcagcctactacgatgccctggcgacctcgggcaccgggcaggaccgcaagttcaccatgcgggaggtggcccgccatattgtgccgaccgacgggtggttggcatgccatgacggtgtctacgacatcaccgagttcatagggaaacatcctggcggcgatgttatttctctcggattgggcagggactccacaatcctggttgagtcgtaccaccctgccggaaggccagacaaggtcatggaaaagtaccgcatcgggacgctccaggaccaccgcacgttctacgactggcaggcctccgcgttctacgccgagctgaagcagcgggtggtgcagacgctaaaggaggccggccaaccgcggcgtgggggcctgtcggtcaaagcggcgctggtcatggcagcgttcgcggcgtcgttctacctcatggtgacccagggatccttcttctgggccgccgtctggggcctcgccggctcccacattggcctcagcatccagcacgacgggaaccacggggctttcagtaagagtggtcggctgaaccgcctcgcaggctggggcatggacgttatcggggcctcctcgacggcctgggagtaccagcacgtcatcgggcaccaccagtacaccaacctggtatcggatcccgagttcgcgctgcctgagaacgacccggacgtcttcggcacctatccgctgatgcggatgcatccggacaccccttggaagccgcaccaccagctgcagcatatgtacgcgttcccgctgttcgccctgatgaccatcagcaaggtcatcatcagtgacttcacgttctgcctcgccaagcggcgcgggccgatcgacttctccgccaggctcgtgccacttgaggggcagatgctcttctggggcgcgaagatcatggggttcctgatgcagatagtcctgccgtgctatctgcatggcatcgcccatgggctggcgctgttcatcacagcccacctggtgtcgggagagtacctcgcggtctgcttcatcatcaaccacatctctgagtcatgcgactatttgaatccaagttccgtcatcgctgcgcggcggacggaaatgctgaagcaggcggagcaggaggccaaggcaaagcagaagcaccccaccccaccgcccaacgactgggccgcgtctcaggtactgtgctgcgtaaactggcgctctggtggatatttctcaaaccacctctcaggcgggctgaaccaccagatcgagcaccacctcttccccagcatctcacatgcgaactatccgaccattgccccagttgtgaaaggggtgtgcgaggagtacggcctcccctacaagaactactcccagttctctgacgctatctatggaatggtggagcacctcagggcgatgggcacgaagcccgaagacaacggcaagctggcgccactgccgggctccctggaggacgtgtgcccggtcctgagtgccgccgttgctgcccaacctgacggaagcaccgacggcagcgctgcgggttgtccagcagtagccacactggca

SEQ ID NO: 18 - protein, 841 aa, Euglena anabaena
MAEGKSDGPVVTLQSMWKPLALMAVDVGILVNVRRKAFTEFDGHSNVFADPVYIPFVMNLFYLTMIFAGCRWMKTREPFEIKSYMFAYNAYQTMMNFLIVVGFMYEVHSTGMRYWGSRIDTSTKGLGLGFLIYAHYHNKYVEYVDTLFMILRKKNNQISFLHVYHHSLLTWAWWAVVYWAPGGDAWFGACYNSFIHVLMYSYYLFATFGIRCPWKKMLTQLQMVQFCFCFAHAMYVGWLGHEVYPRWLTALQAFVMLNMLVLFGNFYMKSYSKASKLEPASPVSPASLAQKPFENAKVKPGGPGKPSEIASLPPPIRPVGNPPAAYYDALATSGTGQDRKFTMREVARHIVPTDGWLACHDGVYDITEFIGKHPGGDVISLGLGRDSTILVESYHPAGRPDKVMEKYRIGTLQDHRTFYDWQASAFYAELKQRVVQTLKEAGQPRRGGLSVKAALVMAAFAASFYLMVTQGSFFWAAVWGLAGSHIGLSIQHDGNHGAFSKSGRLNRLAGWGMDVIGASSTAWEYQHVIGHHQYTNLVSDPEFALPENDPDVFGTYPLMRMHPDTPWKPHHQLQHMYAFPLFALMTISKVIISDFTFCLAKRRGPIDFSARLVPLEGQMLFWGAKIMGFLMQIVLPCYLHGIAHGLALFITAHLVSGEYLAVCFIINHISESCDYLNPSSVIAARRTEMLKQAEQEAKAKQKHPTPPPNDWAASQVLCCVNWRSGGYFSNHLSGGLNHQIEHHLFPSISHANYPTIAPVVKGVCEEYGLPYKNYSQFSDAIYGMVEHLRAMGTKPEDNGKLAPLPGSLEDVCPVLSAAVAAQPDGSTDGSAAGCPAVATLA

SEQ ID NO: 19 - DNA, 2523 bp, Euglena anabaena
atggccgaaggcaagagcgatgggcctgtggtgacccttcaaagcatgtggaaaccgcttgctctgatggcaatagatgtcggcatattggtcaatgtccgccgcaaggctttcactgagtttgatgggcacagcaacgttttcgcagatccagtttacattccatttgtgatgaatctcttctacttgaccatgatctttgctgggtgccgttggatgaagactcgcgaaccctttgagatcaagtcatatatgtttgcatacaatgcatatcagacaatgatgaacttcctcattgtcgtcgggttcatgtatgaggtgcacagcacagggatgcgatattgggggtccaggatcgacacctccaccaagggcttgggcctcggtttcctgatctatgcccactaccacaacaaatacgtggagtatgtcgacacgttgttcatgatcctgcgcaagaaaaacaaccagatctcgttcctccacgtctaccaccattcgcttttgacttgggcctggtgggccgtggtctactgggccccaggaggagatgcctggttcggagcatgctacaattccttcatccacgtgctgatgtactcctactacctgtttgcaacgtttggcatcaggtgcccctggaagaagatgctgacccagttgcagatggtgcaattctgcttctgctttgcccacgcgatgtatgttggatggctgggccatgaagtctacccgcgctggttgacggcgctacaggcatttgtgatgctaaacatgctggtgctgttcggcaacttctacatgaagtcgtactccaaggccagcaagctggagccggcctcccccgtgtcccccgcctccctcgcccagaagccgttcgagaacgccaaggtgaagcctgggggccccggcaagccaagcgagattgcgtcgctgccaccgccaattcgaccagtcgggaacccacctgcagcctactacgatgccctggcgacctcgggcaccgggcaggaccgcaagttcaccatgcgggaggtggcccgccatattgtgccgaccgatgggtggttggcgtgccatgacggtgtctacgacatcaccgagttcatagggaaacatcctggcggcgatgttatttctctcggattgggcagggactccacaatcctggttgagtcgtaccaccctgccggaaggccagacaaggtcatggaaaagtaccgcatcgggacgctccaggaccaccgcacgttctacgactggcaggcctccgcgttctacgccgagctgaagcagcgggtggtgcagacgctaaaggaggccggccaaccgcggcgtgggggcctgtcggtcaaagcggcgctggtcatggcggcgttcgcagcgtcgttctacctcatggtgacccagggatccttcttctgggccgccgtctggggcctcgccggctcccacattggcctcagcatccagcacgacgggaaccacggggctttcagtaagagtggtcggctgaaccgcctcgcgggctggggcatggacgtcatcggggcctcctcgacggcctgggagtaccagcacgtcatcgggcaccaccagtacaccaacctggtatcggatcccgagttcgcgctgcctgagaacgacccggacgtcttcggcacctatccgctgatgcggatgcatccggacaccccttggaagccgcaccaccagctgcagcatgtgtacgcgttcccgctgttcgccctgatgaccatcagcaaggtcatcatcagcgacttcacgttctgcctcgccaagcggcgcgggccgatcgacttctccgccaggctcgtgccacttgaggggcagatgctcttctggggggcgaagatcatggggttcctgatgcagatagtcctgccgtgctatctgcatggcatcgcccatgggctggcgctgttcatcacagcccacctggtgtcgggagagtacctcgcggtctgcttcatcatcaaccacatctctgagtcatgcgactatttgaatccaagttccgtcatcgctgcgcggaggacggaaatgctgaagcaggcggagcaggaggccaaggcaaagcagaagcaccccaccccaccgcccaacgactgggccgcgtctcaggtactgtgctgcgtaaactggcgctctggtggctatttctcaaaccacctctcaggcgggctgaaccaccagatcgagcaccacctcttccccagcatctcacatgcgaactatccgaccattgccccagttgtgaaaggggtgtgcgaggagtacggcctcccctacaagaactactcccagttctccgacgctctctatggaatggtggagcacctcagggcgatgggcacgaagccggcagacaacgacaagctggcgcccaccgcgggctccctggaggacgtgtgcccggtcttgagcgccgccgttgctgcccaacctgacggaagcaccgacggcagcgctgcgggttgtccagcagtagccacactggca

SEQ ID NO: 20 - protein, 841 aa, Euglena anabaena
MAEGKSDGPVVTLQSMWKPLALMAIDVGILVNVRRKAFTEFDGHSNVFADPVYIPFVMNLFYLTMIFAGCRWMKTREPFEIKSYMFAYNAYQTMMNFLIVVGFMYEVHSTGMRYWGSRIDTSTKGLGLGFLIYAHYHNKYVEYVDTLFMILRKKNNQISFLHVYHHSLLTWAWWAVVYWAPGGDAWFGACYNSFIHVLMYSYYLFATFGIRCPWKKMLTQLQMVQFCFCFAHAMYVGWLGHEVYPRWLTALQAFVMLNMLVLFGNFYMKSYSKASKLEPASPVSPASLAQKPFENAKVKPGGPGKPSEIASLPPPIRPVGNPPAAYYDALATSGTGQDRKFTMREVARHIVPTDGWLACHDGVYDITEFIGKHPGGDVISLGLGRDSTILVESYHPAGRPDKVMEKYRIGTLQDHRTFYDWQASAFYAELKQRVVQTLKEAGQPRRGGLSVKAALVMAAFAASFYLMVTQGSFFWAAVWGLAGSHIGLSIQHDGNHGAFSKSGRLNRLAGWGMDVIGASSTAWEYQHVIGHHQYTNLVSDPEFALPENDPDVFGTYPLMRMHPDTPWKPHHQLQHVYAFPLFALMTISKVIISDFTFCLAKRRGPIDFSARLVPLEGQMLFWGAKIMGFLMQIVLPCYLHGIAHGLALFITAHLVSGEYLAVCFIINHISESCDYLNPSSVIAARRTEMLKQAEQEAKAKQKHPTPPPNDWAASQVLCCVNWRSGGYFSNHLSGGLNHQIEHHLFPSISHANYPTIAPVVKGVCEEYGLPYKNYSQFSDALYGMVEHLRAMGTKPADNDKLAPTAGSLEDVCPVLSAAVAAQPDGSTDGSAAGCPAVATLA

SEQ ID NO: 21 - DNA, 2523 bp, Euglena anabaena
atggccgaaggcaagagcgatgggcctgtggtgacccttcaaagcatgtggaaaccgcttgctttgatggcagtagatgtcggcatattggtcaatgtccgccgcaaggctttcactgagtttgatgggcacagcaacgtttttgcagatccagtttacattccatttgtgatgaatctcttctacttgaccatgatctttgctgggtgccgttggatgaagactcgcgagccctttgagatcaagtcatatatgtttgcatacaatgcatatcagacaatgatgaacttcctcattgtcgtcgggttcatgtatgaggtgcacagcacagggatgcgatattgggggtccaggatcgacacctccaccaagggcttgggcctcggtttcctgatctatgcccactaccacaacaaatatgtggagtatgtcgacacgttgttcatgatcctgcgcaagaaaaacaaccagatctcgttcctccacgtctaccaccattcgcttttgacttgggcctggtgggccgtggtctactgggccccgggaggagatgcctggttcggagcatgctacaattccttcatccacgtgctgatgtactcctactacctgtttgcaacgtttggcatcaggtgcccctggaagaagatgctgacccagttgcagatggtgcaattctgcttctgctttgcccacgcgatgtatgttggatggctgggccatgaagtctacccgcgctggttgacggcgctgcaggcatttgtgatgctaaacatgctggtgctgttcggcaacttctacatgaagtcgtactccaaggccagcaagctggagccggcctcccccgcgtcccccgcctccctcgcccagaaaccgttcgagaacgccaaggtgaagcctgggggccccggcaagccaagcgagattgcgtcgctgccaccgccaattcgaccagtcgggaacccacctgcagcctactacgatgccctggcgacctcgggcaccgggcaggaccgcaagttcaccatgcgggaggtggcccgccatattgtgccgaccgacgggtggttggcatgccatgacggtgtctacgacatcaccgagttcatagggaaacatcctggcggcgatgttatttctctcggattgggcagggactccacaatcctggttgagtcgtaccaccctgccggaaggccagacaaggtcatggaaaagtaccgcatcgggacgctccaggaccaccgcacgttctacgactggcaggcctccgcgttctacgccgagctgaagcagcgggtggtgcagacgctaaaggaggccggccaaccgcggcgtgggggcctgtcggtcaaagcggcgctggtcatggcagcgttcgcggcgtcgttctacctcatggtgacccagggatccttcttctgggccgccgtctggggcctcgccggctcccacattggcctcagcatccagcacgacgggaaccacggggctttcagtaagagtggtcggctgaaccgcctcgcaggctggggcatggacgttatcggggcctcctcgacggcctgggagtaccagcacgtcatcgggcaccaccagtacaccaacctggtatcggatcccgagttcgcgctgcctgagaacgacccggacgtcttcggcacctatccgctgatgcggatgcatccggacaccccttggaagccgcaccaccagctgcagcatatgtacgcgttcccgctgttcgccctgatgaccatcagcaaggtcatcatcagtgacttcacgttctgcctcgccaagcggcgcgggccgatcgacttctccgccaggctcgtgccacttgaggggcagatgctcttctggggcgcgaagatcatggggttcctgatgcagatagtcctgccgtgctatctgcatggcatcgcccatgggctggcgctgttcatcacagcccacctggtgtcgggagagtacctcgcggtctgcttcatcatcaaccacatctctgagtcatgcgactatttgaatccaagttccgtcatcgctgcgcggcggacggaaatgctgaagcaggcggagcaggaggccaaggcaaagcagaagcaccccaccccaccgcccaacgactgggccgcgtctcaggtactgtgctgcgtaaactggcgctctggtggatatttctcaaaccacctctcaggcgggctgaaccaccagatcgagcaccacctcttccccagcatctcacatgcgaactatccgaccattgccccagttgtgaaaggggtgtgcgaggagtacggcctcccctacaagaactactcccagttctctgacgctatctatggaatggtggagcacctcagggcgatgggcacgaagcccgaagacaacggcaagctggcgccactgccgggctccctggaggacgtgtgcccggtcctgagtgccgccgttgctgcccaacctgacggaagcaccgacggcagcgctgcgggttgtccagcagtagccacactggca

SEQ ID NO: 22 - protein, 841 aa, Euglena anabaena
MAEGKSDGPVVTLQSMWKPLALMAVDVGILVNVRRKAFTEFDGHSNVFADPVYIPFVMNLFYLTMIFAGCRWMKTREPFEIKSYMFAYNAYQTMMNFLIVVGFMYEVHSTGMRYWGSRIDTSTKGLGLGFLIYAHYHNKYVEYVDTLFMILRKKNNQISFLHVYHHSLLTWAWWAVVYWAPGGDAWFGACYNSFIHVLMYSYYLFATFGIRCPWKKMLTQLQMVQFCFCFAHAMYVGWLGHEVYPRWLTALQAFVMLNMLVLFGNFYMKSYSKASKLEPASPASPASLAQKPFENAKVKPGGPGKPSEIASLPPPIRPVGNPPAAYYDALATSGTGQDRKFTMREVARHIVPTDGWLACHDGVYDITEFIGKHPGGDVISLGLGRDSTILVESYHPAGRPDKVMEKYRIGTLQDHRTFYDWQASAFYAELKQRVVQTLKEAGQPRRGGLSVKAALVMAAFAASFYLMVTQGSFFWAAVWGLAGSHIGLSIQHDGNHGAFSKSGRLNRLAGWGMDVIGASSTAWEYQHVIGHHQYTNLVSDPEFALPENDPDVFGTYPLMRMHPDTPWKPHHQLQHMYAFPLFALMTISKVIISDFTFCLAKRRGPIDFSARLVPLEGQMLFWGAKIMGFLMQIVLPCYLHGIAHGLALFITAHLVSGEYLAVCFIINHISESCDYLNPSSVIAARRTEMLKQAEQEAKAKQKHPTPPPNDWAASQVLCCVNWRSGGYFSNHLSGGLNHQIEHHLFPSISHANYPTIAPVVKGVCEEYGLPYKNYSQFSDAIYGMVEHLRAMGTKPEDNGKLAPLPGSLEDVCPVLSAAVAAQPDGSTDGSAAGCPAVATLA

SEQ ID NO: 23 - DNA, 2442 bp, Euglena anabaena
atggccgaaggcaagagcgatgggcctgtggtgacccttcaaagcatgtggaaaccgcttgctttgatggcagtagatgtcggcatattggtcaacgtccgccgcaaggctttcactgagtttgatgggcacagcaacgtttttgcagatcccgtttacattccatttgtgatgaatctcttctacttgaccatgatctttgctgggtgccgttggatgaagactcgcgaaccctttgagatcaagtcatatatgtttgcatacaatgcatatcagacgatgatgaacttcctcattgtcgtcgggttcatgtatgaggtgcacagcacagggatgcggtattgggggtccaggatcgacacctccaccaagggcttgggcctcggtttcctgatctatgcccactaccacaacaaatacgtggagtatgtcgacacgttgttcatgatcctgcgcaagaaaaacaaccagatctcgttcctccacgtctaccaccattcgcttttgacttgggcctggtgggccgtggtctactgggccccaggaggagatgcctggttcggagcatgctacaattcctttatccacgtgctgatgtactcctactacctgtttgcaacgtttggcatcaggtgcccctggaagaagatgctgacccagttgcagatggtgcaattctgcttctgctttgcccacgcgatgtatgttggatggctgggccatgaagtctacccgcgctggttgacggcgctacaggcatttgtgatgctaaacatgctggtgctgttcggcaacttctacatgaagtcgtactccaaggccagcaagctggagccggcctcccccgtgtcccccgcctccctcgcccagaagccgttcgagaacgccaaggtgaagcctgggggccccggcaagccaagcgagattgcgtcgctgccaccgccaattcgaccagtcgggaacccacctgcagcctactacgatgccctggcgacctcgggcaccgggcaggaccgcaagttcaccatgcgggaggtggcccgccatattgtgccgaccgatgggtggttggcgtgccatgacggtgtctacgacatcaccgagttcatagggaaacatcctggcggcgatgttatttctctcggattgggcagggactccacaatcctggttgagtcgtaccaccctgccggaaggccagacaaggtcatggaaaagtaccgcatcgggacgctccaggaccaccgcacgttctacgactggcaggcctccgcgttctacgccgagctgaagcagcgggtggtgcagacgctaaaggaggccggccaaccgcggcgtgggggcctgtcggtcaaagcggcgctggtcatggcggcgttcgcagcgtcgttctacctcatggtgacccagggatccttcttctgggccgccgtctggggcctcgccggctcccacattggcctcagcatccagcacgacgggaaccacggggctttcagtaagagtggtcggctgaaccgcctcgcgggctggggcatggacgtcatcggggcctcctcgacggcctgggagtaccagcacgtcatcgggcaccaccagtacaccaacctggtatcggatcccgagttcgcgctgcctgagaacgacccggacgtcttcggcacctatccgctgatgcggatgcatccggacaccccttggaagccgcaccaccagctgcagcatatgtacgcgttcccgctgttcgccctgatgaccatcagcaaggtcatcatcagcgacttcacgttctgcctcgccaagcggcgcgggccgatcgacttctccgccaggctcgtgccacttgaggggcagatgctcttctggggggcgaagatcatggggttcctgatgcagatagtcctgccgtgctatctgcatggcatcgcccatgggctggcgctgttcatcacagcccacctggtgtcgggagagtacctcgcggtctgcttcatcatcaaccacatctctgagtcatgcgactatttgaatccaagttccgtcatcgctgcgcggcggacggaaatgctgaagcaggcggagcaggaggccaaggcaaagcagaagcaccccaccccaccgcccaacgactgggccgcgtctcaggtactgtgctgcgtaaactggcgctctggtggatatttctcaaaccacctctcaggcgggctgaaccaccagatcgagcaccacctcttccccagcatctcacatgcgaactatccgaccattgccccagttgtgaaaagggtgtgcgaggagtacggcctcccctacaagaactactcccagttctctgacctctctatggaatggtggagcacctcagggcgatgggcacgaagccggcagacaacgacaagctggcgccaccactgggctccctggaggacgtgtgcccggtcc

SEQ ID NO: 24 - protein, 814 aa, Euglena anabaena
MAEGKSDGPVVTLQSMWKPLALMAVDVGILVNVRRKAFTEFDGHSNVFADPVYIPFVMNLFYLTMIFAGCRWMKTREPFEIKSYMFAYNAYQTMMNFLIVVGFMYEVHSTGMRYWGSRIDTSTKGLGLGFLIYAHYHNKYVEYVDTLFMILRKKNNQISFLHVYHHSLLTWAWWAVVYWAPGGDAWFGACYNSFIHVLMYSYYLFATFGIRCPWKKMLTQLQMVQFCFCFAHAMYVGWLGHEVYPRWLTALQAFVMLNMLVLFGNFYMKSYSKASKLEPASPVSPASLAQKPFENAKVKPGGPGKPSEIASLPPPIRPVGNPPAAYYDALATSGTGQDRKFTMREVARHIVPTDGWLACHDGVYDITEFIGKHPGGDVISLGLGRDSTILVESYHPAGRPDKVMEKYRIGTLQDHRTFYDWQASAFYAELKQRVVQTLKEAGQPRRGGLSVKAALVMAAFAASFYLMVTQGSFFWAAVWGLAGSHIGLSIQHDGNHGAFSKSGRLNRLAGWGMDVIGASSTAWEYQHVIGHHQYTNLVSDPEFALPENDPDVFGTYPLMRMHPDTPWKPHHQLQHMYAFPLFALMTISKVIISDFTFCLAKRRGPIDFSARLVPLEGQMLFWGAKIMGFLMQIVLPCYLHGIAHGLALFITAHLVSGEYLAVCFIINHISESCDYLNPSSVIAARRTEMLKQAEQEAKAKQKHPTPPPNDWAASQVLCCVNWRSGGYFSNHLSGGLNHQIEHHLFPSISHANYPTIAPVVKRVCEEYGLPYKNYSQFSDLSMEWWSTSGRWARSRQTTTSWRHHWAPWRTCARS

SEQ ID NO: 25 - DNA, 777 bp, Euglena gracilis
  misc_feature (1)..(777): delta-9 elongase
atggaggtggtgaatgaaatagtctcaattgggcaggaagttttacccaaagttgattatgcccaactctggagtgatgccagtcactgtgaggtgctttacttgtccatcgcatttgtcatcttgaagttcactcttggcccccttggtccaaaaggtcagtctcgtatgaagtttgttttcaccaattacaaccttctcatgtccatttattcgttgggatcattcctctcaatggcatatgccatgtacaccatcggtgttatgtctgacaactgcgagaaggcttttgacaacaacgtcttcaggatcaccacgcagttgttctatttgagcaagttcctggagtatattgactccttctatttgccactgatgggcaagcctctgacctggttgcaattcttccatcatttgggggcaccgatggatatgtggctgttctataattaccgaaatgaagctgtttggatttttgtgctgttgaatggtttcatccactggatcatgtacggttattattggaccagattgatcaagctgaagttccccatgccaaaatccctgattacatcaatgcagatcattcaattcaatgttggtttctacattgtctggaagtacaggaacattccctgttatcgccaagatgggatgaggatgtttggctggttcttcaattacttttatgttggcacagtcttgtgtttgttcttgaatttctatgtgcaaacgtatatcgtcaggaagcacaagggagccaaaaagattcagtga

SEQ ID NO: 26 - DNA, 1260 bp, Tetruetreptia pomquetensis CCMP1491
  misc_feature (1)..(1260): U.S. Patent Application No. 11/876,115 (filed October 22, 2007)
atgtctcctaagcggcaagctctgccaatcacaattgatggcgcaacttatgatgtgtctgcttgggtcaatcaccaccctggaggagctgacattatcgagaactatcgcaaccgcgatgcgaccgatgtcttcatggtgatgcactctcaagaagccgtcgccaagttgaagagaatgcctgttatggagccttcctctcctgacacacctgttgcacccaagcctaagcgtgatgagccccaggaggatttccgcaagttgcgggaggaattcatctccaagggtatgttcgagacgagtttcctttggtatttttacaagacttcaactaccgtcggtttgatggtcctttccatcttgatgaccgtgtacacgaattggtatttcaccgctgctttggttcttggcgtgtgctaccaacagctaggctggttgtcccacgactattgccatcaccaggttttcacaaaccgcaagattaacgacgctttcggtctctttttcggtaacgtgatgcagggatactcacagacttggtggaaggataggcacaatggtcaccatgccgccaccaatgtggtcggccatgacccagatattgataacctccccatcctggcttggtctcccgaagatgtcaagagggctactccttcgactcggaatctcatcaagtaccagcagtactacttcattcccaccattgcatcccttaggttcatctggtgcctccaatccatcggcggcgtcatgtcctacaagagcgaggagaggaacctgtactacaagcgccagtacactaaggaggcgattggtctggccctccactgggtgctcaaggccactttctattgcagtgccatgcctagctttgccaccggtttgggatgcttcttgatctccgagctgctcggaggatttggcattgccatcgttgtgtttctgaatcactatcctttggacaaggttgaggagactgtctgggatgagcacgggttcagcgccagccagatccacgagacgttgaacattaagcccggccttctcaccgattgggtctttggtggtctcaactaccagattgagcaccacttgtggcccaacatgcccaggcacaacctcacggcagcttccctggaggtgcagaagttgtgcgccaagcacaacctgccctacagggccccagccatcatccccggggttcagaaattggtcagcttcttaggcgagattgcccagctggctgctgtccctgaa

SEQ ID NO: 27 - DNA, 4300 bp, Artificial Sequence
  plasmid pLF114-10
taatacgactcactatagggcgaattgggcccgacgtcgcatgctcccggccgccatggcggccgcgggaattcgattggcggccgcaccatgtctcctaagcggcaagctctgccaatcacaattgatggcgcaacttatgatgtgtctgcttgggtcaatcaccaccctggaggagctgacattatcgagaactatcgcaaccgcgatgcgaccgatgtcttcatggtgatgcactctcaagaagccgtcgccaagttgaagagaatgcctgttatggagccttcctctcctgacacacctgttgcacccaagcctaagcgtgatgagccccaggaggatttccgcaagttgcgggaggaattcatctccaagggtatgttcgagacgagtttcctttggtatttttacaagacttcaactaccgtcggtttgatggtcctttccatcttgatgaccgtgtacacgaattggtatttcaccgctgctttggttcttggcgtgtgctaccaacagctaggctggttgtcccacgactattgccatcaccaggttttcacaaaccgcaagattaacgacgctttcggtctctttttcggtaacgtgatgcagggatactcacagacttggtggaaggataggcacaatggtcaccatgccgccaccaatgtggtcggccatgacccagatattgataacctccccatcctggcttggtctcccgaagatgtcaagagggctactccttcgactcggaatctcatcaagtaccagcagtactacttcattcccaccattgcatcccttaggttcatctggtgcctccaatccatcggcggcgtcatgtcctacaagagcgaggagaggaacctgtactacaagcgccagtacactaaggaggcgattggtctggccctccactgggtgctcaaggccactttctattgcagtgccatgcctagctttgccaccggtttgggatgcttcttgatctccgagctgctcggaggatttggcattgccatcgttgtgtttctgaatcactatcctttggacaaggttgaggagactgtctgggatgagcacgggttcagcgccagccagatccacgagacgttgaacattaagcccggccttctcaccgattgggtctttggtggtctcaactaccagattgagcaccacttgtggcccaacatgcccaggcacaacctcacggcagcttccctggaggtgcagaagttgtgcgccaagcacaacctgccctacagggccccagccatcatccccggggttcagaaattggtcagcttcttaggcgagattgcccagctggctgctgtccctgaatgagcggccgcaatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattg

SEQ ID NO: 28 - DNA, 31 bp, Artificial Sequence
  oligonucleotide MGW511
gaattcgcggccgctcactgccagggagtcg

SEQ ID NO: 29 - protein, 11 aa, Artificial Sequence
  conserved motif at C-terminal for C20elo domains
VLFXXFYXXXY

SEQ ID NO: 30 - protein, 4 aa, Euglena gracilis
KNGK

SEQ ID NO: 31 - protein, 5 aa, Euglena gracilis
PENGA

SEQ ID NO: 32 - protein, 7 aa, Euglena gracilis
PCENGTV

SEQ ID NO: 33 - DNA, 54 bp, Euglena gracilis
cccgcccgtccagctggactcccgccggccacgtactacgactccctggcagtg

SEQ ID NO: 34 - protein, 18 aa, Euglena gracilis
PARPAGLPPATYYDSLAV

SEQ ID NO: 35 - DNA, 54 bp, Euglena gracilis
cccacccgtccagctggacccccgccggccacgtactacgactccctggcagtg

SEQ ID NO: 36 - protein, 18 aa, Euglena gracilis
PTRPAGPPPATYYDSLAV

SEQ ID NO: 37 - DNA, 2379 bp, Euglena gracilis
atggcggatagcccagtcatcaacctcagcaccatgtggaaacccctttcactgatggctttggaccttgccgttttgggacatgtctggaagcaggcacaacaggagggcagcatttcggcctatgctgattctgtttggactcctctcattatgtccggtttatacttatcaatgatcttcgtggggtgccgctggatgaagaaccgtgaaccctttgagatcaaaacatacatgtttgcgtataacctgtatcagaccttgatgaacctttgcatcgtgttgggattcttgtaccaggtgcatgccactgggatgcgcttttggggaagtggtgtcgaccgaagcccaaaaggtttgggcattggcttcttcatttatgcccactaccacaacaagtatgtggaatattttgatacactttttatggtgctgcgaaagaagaacaaccagatttctttccttcacgtgtatcatcatgccctgttgacatgggcttggtttgctgttgtgtatttcgcacctggaggtgatggctggtttggagcttgctacaattcttccatccatgtcctgatgtactcttactacttgcttgcaacttttggcatcagttgcccttggaagaagatcttgacacagctccagatggttcaattctgtttctgttttacacattccatttatgtgtggatttgcgggtcagagatctacccacggcctctgactgctttgcagtcgttcgtgatggtcaatatgttggtgctgtttggcaatttctatgtcaagcaatactcccaaaagaacggcaagccggagaacggagccacccctgagaacggagcgaagccgcaaccttgcgagaacggcacggtggaaaagcgagagaatgacaccgccaacgttcggcccgcccgtccagctggactcccgccggccacgtactacgactccctggcagtgtcggggcagggcaaggagcggctgttcaccaccgatgaggtgaggcggcacatcctccccaccgatggctggctgacgtgccacgaaggagtctacgatgtcactgatttccttgccaagcaccctggtggcggtgtcatcacgctgggccttggaagggactgcacaatcctcgtcgagtcataccaccctgctgggcgcccggacaaggtgatggagaagtaccgcattggtacgctgcaggaccccaagacgttctatgcttggggagagtccgatttctaccctgagttgaagcgccgggcccttgcaaggctgaaggaggctggtcaggcgcggcgcggcggccttggggtgaaggccctcctggtgctcaccctcttcttcgtgtcgtggtacatgtgggtggcccacaagtccttcctctgggccgccgtctggggcttcgccggctcccacgtcgggctgagcatccagcacgacggcaaccacggcgcgttcagccgcagcacactggtgaaccgcctggcggggtggggcatggacttgatcggcgcgtcgtcaacggtgtgggagtaccagcacgtcatcggccaccaccagtacaccaacctcgtgtcggacacgctattcagtctgcctgagaacgatccggacgtcttctccagctacccgctgatgcgcatgcacccggatacggcgtggcagccgcaccaccgcttccagcacctgttcgcgttcccactgttcgccctgatgacaatcagcaaggtgctgaccagcgatttcgctgtctgcctcagcatgaagaaggggtccatcgactgctcctccaggctcgtcccactggaggggcagctgctgttctggggggccaagctggcgaacttcctgttgcagattgtgttgccatgctacctccacgggacagctatgggcctggccctcttctctgttgcccaccttgtgtcgggggagtacctcgcgatctgcttcatcatcaaccacatcagcgagtcttgtgagtttatgaatacaagctttcaaaccgccgcccggaggacagagatgcttcaggcagcccatcaggcagcggaggccaagaaggtgaagcccacccctccaccgaacgattgggctgtgacacaggtccaatgctgcgtgaattggagatcaggtggcgtgttggccaatcacctctctggaggcttgaaccaccagatcgagcatcatctgttccccagcatctcgcatgccaactaccccatcatcgcccgtgttgtgaaggaggtgtgcgaggagtatgggttgccgtacaagaactacgtcacgttctgggatgcagtctgtggcatggttcagcacctccggttgatgggtgctccaccggtgccaacgaacggggacaaaaagtca

SEQ ID NO: 38 - DNA, 3668 bp, Artificial Sequence
  Plasmid pLF121-1
gtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcatagatcttttctccatcactgatagggagtggtaaaataactccatcaatgatagagtgtcaacaacatgaccaaaatcccttaacgtgagttacgcgtattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggttacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagggaacgacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagttggtttttttcggtctaaaatggaagcagccaaagaattggtttccatcgtccaagaggagctccccaaggtggactatgcccagctttggcaggatgccagcagctgtgaggtcctttacctctcggtggcattcgtggcgatcaagttcatgctgcgcccactggacctgaagcgccaggccaccttgaagaagctgttcacagcatacaacttcctcatgtcgatctattcctttggctccttcctggccatggcctatgccctatcagtaactggcatcctctccggcgactgtgagacggcgttcaacaacgatgtgttcaggatcacaactcagctgttctacctcagcaagttcgtagagtacatcgactccttctaccttccccttatggacaagccactgtcgttccttcagttcttccatcatttgggggcccccattgacatgtggctattctacaaataccgcaacgaaggagtctggatctttgtcctgttgaatgggttcattcactggatcatgtacggttactattggacgcggctcatcaagctgaacttccccatgcccaagaacctgatcacctccatgcagatcatccagttcaatgtcgggttctacatcgtctggaagtaccgcaatgtgccatgctaccgccaggatgggatgcgcatgtttgcctggatcttcaactactggtatgtcgggacggtcttgctgctgttcctcaacttttacgtgcagacgtacatccggaagccgaggaagaaccgagggaagaaggagtaggccacatggcgcctgcgctggaggaaacggtacgctcggatggtgcactgcacttgcactccgccgtttctagcctcccctcgctctaaccactgcggcatgcctgcttgaggcgtgacgttgcctcgtatgatacagtttacacccttcccacagcccacggagctggtgactgtttccagcgtctgcagatcattgatctggtgcaatgtgcacagaccaagcccctctaacgtcttgcggtgtaccgctcgacactcactgcaagagacagatggctgagcatgttatagccccttacattctacccttcgtcccaacctgaccgtcacattcnnnnnnnnnnnnnnnnnnnnnnnnnnnnnnnnnnnnnnnnacccaactttctt

SEQ ID NO: 39 - DNA, 774 bp, Euglena anabaena
atggaagcagccaaagaattggtttccatcgtccaagaggagctccccaaggtggactatgcccagctttggcaggatgccagcagctgtgaggtcctttacctctcggtggcattcgtggcgatcaagttcatgctgcgcccactggacctgaagcgccaggccaccttgaagaagctgttcacagcatacaacttcctcatgtcgatctattcctttggctccttcctggccatggcctatgccctatcagtaactggcatcctctccggcgactgtgagacggcgttcaacaacgatgtgttcaggatcacaactcagctgttctacctcagcaagttcgtagagtacatcgactccttctaccttccccttatggacaagccactgtcgttccttcagttcttccatcatttgggggcccccattgacatgtggctattctacaaataccgcaacgaaggagtctggatctttgtcctgttgaatgggttcattcactggatcatgtacggttactattggacgcggctcatcaagctgaacttccccatgcccaagaacctgatcacctccatgcagatcatccagttcaatgtcgggttctacatcgtctggaagtaccgcaatgtgccatgctaccgccaggatgggatgcgcatgtttgcctggatcttcaactactggtatgtcgggacggtcttgctgctgttcctcaacttttacgtgcagacgtacatccggaagccgaggaagaaccgagggaagaaggag

SEQ ID NO: 40 - protein, 258 aa, Euglena anabaena
MEAAKELVSIVQEELPKVDYAQLWQDASSCEVLYLSVAFVAIKFMLRPLDLKRQATLKKLFTAYNFLMSIYSFGSFLAMAYALSVTGILSGDCETAFNNDVFRITTQLFYLSKFVEYIDSFYLPLMDKPLSFLQFFHHLGAPIDMWLFYKYRNEGVWIFVLLNGFIHWIMYGYYWTRLIKLNFPMPKNLITSMQIIQFNVGFYIVWKYRNVPCYRQDGMRMFAWIFNYWYVGTVLLLFLNFYVQTYIRKPRKNRGKKE

SEQ ID NO: 41 - DNA, 31 bp, Artificial Sequence
  EaD9-5Bbs primer
gaagacaccatggaagcagccaaagaattgg

SEQ ID NO: 42 - DNA, 30 bp, Artificial Sequence
  EaD9-3fusion primer
agctggacgggcgggctccttcttccctcg

SEQ ID NO: 43 - DNA, 30 bp, Artificial Sequence
  EgDHAsyn1Link-5fusion primer
cgagggaagaaggagcccgcccgtccagct

SEQ ID NO: 44 - DNA, 852 bp, Artificial Sequence
  EaD9elo1-EgDHAsyn1Link
gaagacaccatggaagcagccaaagaattggtttccatcgtccaagaggagctccccaaggtggactatgcccagctttggcaggatgccagcagctgtgaggtcctttacctctcggtggcattcgtggcgatcaagttcatgctgcgcccactggacctgaagcgccaggccaccttgaagaagctgttcacagcatacaacttcctcatgtcgatctattcctttggctccttcctggccatggcctatgccctatcagtaactggcatcctctccggcgactgtgagacggcgttcaacaacgatgtgttcaggatcacaactcagctgttctacctcagcaagttcgtagagtacatcgactccttctaccttccccttatggacaagccactgtcgttccttcagttcttccatcatttgggggcccccattgacatgtggctattctacaaataccgcaacgaaggagtctggatctttgtcctgttgaatgggttcattcactggatcatgtacggttactattggacgcggctcatcaagctgaacttccccatgcccaagaacctgatcacctccatgcagatcatccagttcaatgtcgggttctacatcgtctggaagtaccgcaatgtgccatgctaccgccaggatgggatgcgcatgtttgcctggatcttcaactactggtatgtcgggacggtcttgctgctgttcctcaacttttacgtgcagacgtacatccggaagccgaggaagaaccgagggaagaaggagcccgcccgtccagctggactcccgccggccacgtactacgactccctggcagtgagcggccgcgaattc

SEQ ID NO: 45 - DNA, 5559 bp, Artificial Sequence
  Plasmid pLF124
ggccgcaagtatgaactaaaatgcacgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaaggaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtaaagcaatggttgaccgtgtgcttagcttcttttattttatttttttatcagcaaagaataaataaaataaaatgagacacttcagggatgtttcaacggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccctagaactagtggatccttcatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactccatggaagcagccaaagaattggtttccatcgtccaagaggagctccccaaggtggactatgcccagctttggcaggatgccagcagctgtgaggtcctttacctctcggtggcattcgtggcgatcaagttcatgctgcgcccactggacctgaagcgccaggccaccttgaagaagctgttcacagcatacaacttcctcatgtcgatctattcctttggctccttcctggccatggcctatgccctatcagtaactggcatcctctccggcgactgtgagacggcgttcaacaacgatgtgttcaggatcacaactcagctgttctacctcagcaagttcgtagagtacatcgactccttctaccttccccttatggacaagccactgtcgttccttcagttcttccatcatttgggggcccccattgacatgtggctattctacaaataccgcaacgaaggagtctggatctttgtcctgttgaatgggttcattcactggatcatgtacggttactattggacgcggctcatcaagctgaacttccccatgcccaagaacctgatcacctccatgcagatcatccagttcaatgtcgggttctacatcgtctggaagtaccgcaatgtgccatgctaccgccaggatgggatgcgcatgtttgcctggatcttcaactactggtatgtcgggacggtcttgctgctgttcctcaacttttacgtgcagacgtacatccggaagccgaggaagaaccgagggaagaaggagcccgcccgtccagctggactcccgccggccacgtactacgactccctggcagtgagc

SEQ ID NO: 46 - DNA, 5559 bp, Artificial Sequence; identical to SEQ ID NO: 45
  Plasmid pKR1177

SEQ ID NO: 47 - DNA, 7916 bp, Artificial Sequence
  Plasmid pKR1179
gatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccttcatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactccatggaagcagccaaagaattggtttccatcgtccaagaggagctccccaaggtggactatgcccagctttggcaggatgccagcagctgtgaggtcctttacctctcggtggcattcgtggcgatcaagttcatgctgcgcccactggacctgaagcgccaggccaccttgaagaagctgttcacagcatacaacttcctcatgtcgatctattcctttggctccttcctggccatggcctatgccctatcagtaactggcatcctctccggcgactgtgagacggcgttcaacaacgatgtgttcaggatcacaactcagctgttctacctcagcaagttcgtagagtacatcgactccttctaccttccccttatggacaagccactgtcgttccttcagttcttccatcatttgggggcccccattgacatgtggctattctacaaataccgcaacgaaggagtctggatctttgtcctgttgaatgggttcattcactggatcatgtacggttactattggacgcggctcatcaagctgaacttccccatgcccaagaacctgatcacctccatgcagatcatccagttcaatgtcgggttctacatcgtctggaagtaccgcaatgtgccatgctaccgccaggatgggatgcgcatgtttgcctggatcttcaactactggtatgtcgggacggtcttgctgctgttcctcaacttttacgtgcagacgtacatccggaagccgaggaagaaccgagggaagaaggagcccgcccgtccagctggactcccgccggccacgtactacgactccctggcagtgagcggccgcaagtatgaactaaaatgcacgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaaggaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtaaagcaatggttgaccgtgtgcttagcttcttttattttatttttttatcagcaaagaataaataaaataaaatgagacacttcagggatgtttcaacg

SEQ ID NO: 48 - DNA, 9190 bp, Artificial Sequence
  Plasmid pKR1183
ggccgcaagtatgaactaaaatgcacgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaaggaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtaaagcaatggttgaccgtgtgcttagcttcttttattttatttttttatcagcaaagaataaataaaataaaatgagacacttcagggatgtttcaacggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccttcatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactccatggaagcagccaaagaattggtttccatcgtccaagaggagctccccaaggtggactatgcccagctttggcaggatgccagcagctgtgaggtcctttacctctcggtggcattcgtggcgatcaagttcatgctgcgcccactggacctgaagcgccaggccaccttgaagaagctgttcacagcatacaacttcctcatgtcgatctattcctttggctccttcctggccatggcctatgccctatcagtaactggcatcctctccggcgactgtgagacggcgttcaacaacgatgtgttcaggatcacaactcagctgttctacctcagcaagttcgtagagtacatcgactccttctaccttccccttatggacaagccactgtcgttccttcagttcttccatcatttgggggcccccattgacatgtggctattctacaaataccgcaacgaaggagtctggatctttgtcctgttgaatgggttcattcactggatcatgtacggttactattggacgcggctcatcaagctgaacttccccatgcccaagaacctgatcacctccatgcagatcatccagttcaatgtcgggttctacatcgtctggaagtaccgcaatgtgccatgctaccgccaggatgggatgcgcatgtttgcctggatcttcaactactggtatgtcgggacggtcttgctgctgttcctcaacttttacgtgcagacgtacatccggaagccgaggaagaaccgagggaagaaggagcccgcccgtccagctggactcccgccggccacgtactacgactccctggcagtgagcggccgcaccatgtctcctaagcggcaagctctgccaatcacaattgatggcgcaacttatgatgtgtctgcttgggtcaatcaccaccctggaggagctgacattatcgagaactatcgcaaccgcgatgcgaccgatgtcttcatggtgatgcactctcaagaagccgtcgccaagttgaagagaatgcctgttatggagccttcctctcctgacacacctgttgcacccaagcctaagcgtgatgagccccaggaggatttccgcaagttgcgggaggaattcatctccaagggtatgttcgagacgagtttcctttggtatttttacaagacttcaactaccgtcggtttgatggtcctttccatcttgatgaccgtgtacacgaattggtatttcaccgctgctttggttcttggcgtgtgctaccaacagctaggctggttgtcccacgactattgccatcaccaggttttcacaaaccgcaagattaacgacgctttcggtctctttttcggtaacgtgatgcagggatactcacagacttggtggaaggataggcacaatggtcaccatgccgccaccaatgtggtcggccatgacccagatattgataacctccccatcctggcttggtctcccgaagatgtcaagagggctactccttcgactcggaatctcatcaagtaccagcagtactacttcattcccaccattgcatcccttaggttcatctggtgcctccaatccatcggcggcgtcatgtcctacaagagcgaggagaggaacctgtactacaagcgccagtacactaaggaggcgattggtctggccctccactgggtgctcaaggccactttctattgcagtgccatgcctagctttgccaccggtttgggatgcttcttgatctccgagctgctcggaggatttggcattgccatcgttgtgtttctgaatcactatcctttggacaaggttgaggagactgtctgggatgagcacgggttcagcgccagccagatccacgagacgttgaacattaagcccggccttctcaccgattgggtctttggtggtctcaactaccagattgagcaccacttgtggcccaacatgcccaggcacaacctcacggcagcttccctggaggtgcagaagttgtgcgccaagcacaacctgccctacagggccccagccatcatccccggggttcagaaattggtcagcttcttaggcgagattgcccagctggctgctgtccctgaatgagc

SEQ ID NO: 49 - DNA, 99 bp, Euglena anabaena
cctgggggccccggcaagccaagcgagattgcgtcgctgccaccgccaattcgaccagtcgggaacccacctgcagcctactacgatgccctggcgacc

SEQ ID NO: 50 - protein, 33 aa, Euglena anabaena
PGGPGKPSEIASLPPPIRPVGNPPAAYYDALAT

(19) United States
(12) Patent Application Publication
(10) Pub. No.: US 2011/0008868 A1
(43) Pub. Date: Jan. 13, 2011

(54) MULTIZYMES

(75) Inventors: Howard Glenn Damude, Hockessin, DE (US); Steven Gutteridge, Wilmington, DE (US); Anthony J. Kinney, Wilmington, DE (US); Michael W. Lassner, Urbandale, IA (US); Daniel L. Siehl, Menlo Park, CA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/922,500

(22) PCT Filed: Apr. 3, 2009
(86) PCT No.: PCT/US09/39373

§ 371 Date: Sep. 14, 2010

Related U.S. Application Data
(60) Provisional application No. 61/041,956, filed on Apr. 3, 2008.

(51) Int. Cl. C12N 9/02; C12N 9/16

(52) U.S. Cl. 435/189; 435/193

(57) ABSTRACT

Multizymes (i.e., single polypeptides having at least two independent and separable enzymatic activities) are disclosed for a variety of applications beyond the synthesis of long chain polyunsaturated fatty acids.

This application claims the benefit of U.S. Provisional Application No. 61/041956, filed Apr. 3, 2008, the entire content of which is herein incorporated by reference.

### FIELD OF THE INVENTION

This invention is in the field of biotechnology. More specifically, this invention pertains to polynucleotide sequences encoding novel multizymes uses outside the area of fatty acid biosynthesis.

### BACKGROUND OF THE INVENTION

Multizymes and their use in the production of long chain polyunsaturated fatty acids constitute the subject matter of Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety.

Multizymes are defined in the related non-provisional filing as comprising a single polypeptide having at least two independent and separable enzymatic activities. The concept of a multizyme originated with a unique DHA synthase gene that was isolated from Euglena and Euglenoid homologs. This DHA synthase was found to be a fusion of a delta-4 desaturase and a C20 condensing enzyme (an Elo-type elongase). This was the first time that two enzymes with very different activities/functions were found to be fused in this way.

Another unique aspect of this DHA synthase gene was the linker region that joined the two genes encoding these enzymes. Initially, multizymes were studied with respect to synthesized long chain polyunsaturated fatty acids. However, it has since been found that this unique linker can be used to make multizymes whose applications extend far beyond the synthesis of long chain polyunsaturated fatty acids.

The present invention focuses on making multizymes with uses extending beyond the realm of fatty acid biosynthesis.

### SUMMARY OF THE INVENTION

The present invention concerns a multizyme comprising a single polypeptide having at least two independent and separable activities wherein the independent and separable activities are joined together using a linker derived from a sequence of a DHA synthase locus of a Euglenoid wherein said linker can be natural or synthetic provided that the multizyme does not comprise (i) a fatty acid desaturase linked to a fatty acid elongae or (ii) a fatty acid desaturase linked to another fatty acid desaturase. In a second embodiment, the multizyme of the invention can be used to increase total flux of all combined reactions of the multizyme when compared with the total flux of the combined reactions when each reaction is produced separately when not linked together as the multizyme.

In a third embodiment, the multizyme of the invention can be used in a reaction sequence to lower an amount of at least one intermediate produced by the multizyme when compared to the amount of substantially the same intermediate produced by each individual enzymatic activity in a reaction sequence when not linked together as a multizyme.

In a fourth embodiment, the multizyme of the invention can be used in a reaction to reduce the extent of any feedback inhibition of at least one enzymatic activity of the multizyme when compared to the amount of feedback inhibition of a corresponding enzymatic activity that occurs when said individual enzymatic activity is not part of a multizyme.

In a fifth embodiment, the multizyme of the invention can be used in a reaction to protect an activity of the multizyme from competitive inhibition by a substrate other than that produced by a preceding enzymatic activity of the multizyme.

In a sixth embodiment, the multizyme of the invention can be used such that a product of a reaction of the multizyme is substantially used in a subsequent reaction of the multizyme.

In a seventh embodiment, the multizyme of the invention can be used such that at least one independent and separable activity of the multizyme is prevented from associating with polypeptide complexes other than the multizyme of which it is a part.

In an eighth embodiment, the multizyme of the invention can be used such that a product of an enzymatic reaction of the multizyme is channeled into a transporter activity which is comprised by the multizyme.

In a ninth embodiment, the multizyme of the invention can be used such that at least one activity of the multizyme that is capable of multiple functions when expressed as an individual polypeptide is restricted to a single function when it is part of the multizyme.

In a tenth embodiment, the multizyme of the invention can be used such that at least two enzymatic activities which when linked together have resistance to an herbicide inhibitor of at least one of the individual activities. For example, the multizyme can comprise a 4-hydroxyphenyl pyruvate dioxygenase linked to an activity that produces hydropxyphenylpyruvate or it can comprises a 4-hydroxyphenyl pyruvate dioxygenase linked to a prephenate dehydrogenase or a bifunctional chorismate mutase.

In an eleventh embodiment, expression of the multizyme of the invention in a transgenic plant confers an increase in seed yield or drought resistance of said plant.

Other embodiments include the following:

a multizyme comprising a 1-hydroxy-2-methyl-2-(E)-butenyl4-diphosphate reductase linked to adenosine phosphate-isopentenyltransferase;

a multizyme comprising an herbicide resistant acetolactate synthase peptide linked to a second herbicide resistant acetolactate synthase peptide;

a multizyme comprising a soluble nitrate reductase linked to a nitrate transporter peptide; or

a multizyme comprising an acyl-ACP-thioesterase protein linked to a beta-ketoacyl-ACP synthetase II protein.

### BRIEF DESCRIPTION OF THE DRAWINGS AND SEQUENCE LISTINGS

The invention can be more fully understood from the following detailed description and the accompanying drawings and Sequence Listing, which form a part of this application.

FIG. 1 is a representative omega-3 and omega-6 fatty acid pathway providing for the conversion of myristic acid through various intermediates to DHA.

FIG. 2 shows a Clustal W alignment between a portion of the coding sequence of EgDHAsyn2 (SEQ ID NO:7), the cDNA sequence of the Euglena gracilis delta-4 desaturase (SEQ ID NO:9) (NCBI Accession No. AY278558 (GI 33466345), locus AY278558, Meyer et al., Biochemistry 42(32):9779-9788 (2003)) and the coding sequence of the Euglena gracilis delta-4 desaturase (SEQ ID NO:10) (Meyer et al., supra). EgDHAsyn2_CDS is nt 621-940 of SEQ ID NO:7, EgD4_cDNA is nt 621-931 of SEQ ID NO:9, and EgD4_CDS is nt 1-184 of SEQ ID NO:7.

FIGS. 3A and 3B show a Clustal W alignment between the amino acid sequence of EgDHAsyn1 (SEQ ID NO:5), EgDHAsyn2 (SEQ ID NO:8) and EgC20elo1 (SEQ ID NO:3).

FIG. 4 shows an alignment of an interior fragment of EgDHAsyn1 (EgDHAsyn1_ NCT; amino acids 253-365 of SEQ ID NO:5) and EgDHAsyn2 (EgDHAsyn2_NCT; amino acids 253-365 of SEQ ID NO:8) spanning both the C20 elongase region and the delta-4 desaturase domain (based on homology) with the C-termini of C20 elongases (EgC20elo1_CT, amino acids 246-298 of SEQ ID NO:3; PavC20elo_CT, amino acids 240-277 of SEQ ID NO:1; OtPUFAelo2_CT, amino acids 256-300 of SEQ ID NO:11; TpPUFAelo2_CT, amino acids 279-358 of SEQ ID NO:26) and the N-termini of delta-4 desaturases (EgD4_NT, amino acids 1-116 of SEQ ID NO:6; TaD4_NT, amino acids 1-47 of SEQ ID NO:13; SaD4_NT, amino acids 1-47 of SEQ ID NO:14; TpD4_NT, amino acids 1-82 of SEQ ID NO:15; IgD4_NT, amino acids 1-43 of SEQ ID NO:16).

FIGS. 5A, 5B and 5C show a Clustal W alignment of the amino acid sequences for EaDHAsyn1 (SEQ ID NO:18), EaDHAsyn2 (SEQ ID NO:20), EaDHAsyn3 (SEQ ID NO:22) and EaDHAsyn4 (SEQ ID NO:24).

FIG. 6. shows a schematic representation of the cytokinin biosynthetic pathway with native enzymes (top) and the cytokinin biosynthetic pathway with a HMBDPR-IPT multizyme (bottom).

FIG. 7 shows the Modified Hoagland's solution-16× concentration for semi-hydroponics maize growth.

FIG. 8 shows the effect of different nitrate concentrations on the growth and development of Gaspe Flint derived maize lines.

FIG. 9 shows a schematic representation of the ARA biosynthetic pathway. LA=linoleic acid, EDA=eicosadienoic acid, JUP=juniperonic acid, HGLA=dihomo-gamma-linolenic acid, ARA=arachidonic acid.

The sequence descriptions summarize the Sequences Listing attached hereto. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

SEQ ID NOs:1-50 are primers, ORFs encoding genes or proteins (or portions thereof), or plasmids, as identified in Table 1.

TABLE 1
Summary Of Nucleic Acid And Protein SEQ ID Numbers

| | Nucleic acid | Protein |
|---|---|---|
| Description and Abbreviation | SEQ ID NO. | SEQ ID NO. |
| Pavlova sp. CCMP459 C20-polyunsaturated fatty | — | 1 (277 AA) |
| acid elongase (GenBank Accession No. AAV33630) | | |
| Euglena gracilis clone eeg1c.pk005.p14.f coding | 2 (897 bp) | 3 (298 AA) |
| sequence (“EgC20elo1”) | | |
| Euglena gracilis clone eeg1c.pk016.e6.f coding | 4 (2382 bp) | 5 (793 AA) |
| sequence (DHA synthase 1 or “EgDHAsyn1”) | | |
| Euglena gracilis delta-4 fatty acid desaturase | — | 6 (541 AA) |
| (GenBank Accession No. AAQ19605) | | |
| Euglena gracilis clone eeg1c-1 coding sequence | 7 (2382 bp) | 8 (793 AA) |
| (DHA synthase 2 or “EgDHAsyn2”) | | |
| Euglena gracilis delta-4 desaturase cDNA | 9 (2569 bp) | — |
| sequence (GenBank Accession No. AY278558) | | |
| Euglena gracilis delta-4 desaturase-coding | 10 (1626 bp) | — |
| sequence (GenBank Accession No. AY278558) | | |
| Ostreococcus tauri PUFA elongase 2 (GenBank | — | 11 (300 AA) |
| Accession No. AAV67798) | | |
| Thalassiosira pseudonana PUFA elongase 2 | — | 12 (358 AA) |
| (GenBank Accession No. AAV67800) | | |
| Thraustochytrium aureum delta-4 desaturase | — | 13 (515 AA) |
| (GenBank Accession No. AAN75707) | | |
| Schizochytrium aggregatum delta-4 desaturase | — | 14 (509 AA) |
| (PCT Publication No. WO 2002/090493) | | |
| Thalassiosira pseuduonana delta-4 fatty acid | — | 15 (550 AA) |
| desaturase (GenBank Accession No. AAX14506) | | |
| Isochrysis galbana delta-4 desaturase (GenBank | — | 16 (433 AA) |
| Accession No. AAV33631) | | |
| Euglena anabaena coding sequence (DHA | 17 (2523 bp) | 18 (841 AA) |
| synthase 1 or “EaDHAsyn1”) | | |
| Euglena anabaena coding sequence (DHA | 19 (2523 bp) | 20 (841 AA) |
| synthase 2 or “EaDHAsyn2”) | | |
| Euglena anabaena coding sequence (DHA | 21 (2523 bp) | 22 (841 AA) |
| synthase 3 or “EaDHAsyn3”) | | |
| Euglena anabaena coding sequence (DHA | 23 (2442 bp) | 24 (814 AA) |
| synthase 4 or “EaDHAsyn4”) | | |
| Euglena gracilis delta-9 elongase (“EgD9elo” or | 25 (777 bp) | — |
| “EgD9e” or EgD9E”) | | |
| Tetruetreptia pomquetensis CCMP1491 delta-8 | 26 (1260 bp) | — |
| desaturase (“TpomD8”) | | |
| Plasmid pLF114-10 | 27 (4300 bp) | — |
| MWG511 primer | 28 | — |
| Conserved motif at C-terminal for C20 elongase domains | — | 29 (11 AA) |
| NG motif located at the C-terminus of each of the | — | 30 |
| C20 elongase domains of EgDHAsyn1, EgDHAsyn2 | | |
| and EgC20elo1 | | |
| PENGA motif located at the C-terminus of each of | — | 31 |
| the C20 elongase domains of EgDHAsyn1, | | |
| EgDHAsyn2 and EgC20elo1 | | |
| PCENGTV motif located at the C-terminus of each | | 32 |
| of the C20 elongase domains of EgDHAsyn1, | | |
| EgDHAsyn2 and EgC20elo1 | | |
| Euglena gracilis EgDHAsyn1 proline-rich linker | 33 (54 bp) | 34 (18 AA) |
| Euglena gracilis EgDHAsyn2 proline-rich linker | 35 (54 bp) | 36 (18 AA) |
| Euglena gracilis EgDHAsyn1* (internal Ncol site removed) | 37 (2379 bp) | — |
| Plasmid pLF121-1 | 38 (3668 bp) | — |
| Euglena anabaena delta-9 elongase 1 (“EaD9Elo1”); | 39 (774 bp) | 40 (258 AA) |
| also referred to herein as “EaD9E” and “EaD9e” | | |
| EaD9-5Bbs primer | 41 | — |
| EaD9-3fusion primer | 42 | — |
| EgDHAsyn1Link-5fusion primer | 43 | — |
| EaD9Elo1-EgDHAsyn1Link linker | 44 (852 bp) | — |
| Plasmid pLF124 | 45 (5559 bp) | — |
| Plasmid pKR1177 | 46 (5559 bp) | — |
| Plasmid pKR1179 | 47 (7916 bp) | — |
| Plasmid pKR1183 | 48 (9190 bp) | — |
| Euglena anabaena EaDHAsyn1 proline-rich linker | 49 (99 bp) | 50 (33 AA) |

### DETAILED DESCRIPTION OF THE INVENTION

The disclosure of each reference set forth herein is hereby incorporated by reference in its entirety.

As was discussed above, the concept of a multizyme originated with a unique DHA synthase gene that was isolated from Euglena and Euglenoid homologs. This DHA synthase was found to be a fusion of a delta-4 desaturase and a C20 condensing enzyme (an Elo-type elongase). This was the first time that two enzymes with very different activities/functions were found to be fused in this way.

Accordingly, the present invention is concerned with making a variety of multizymes using the linker(s) described herein for a plethora of applications extending beyond fatty acid biosynthesis and, more particularly, beyond their use in the production of long chain polyunsaturated fatty acids that constitutes the subject matter of Applicants' Assignee's application Ser. No. 12/061,738 (filed Apr. 3, 2008 which published Oct. 16, 2008; Attorney Docket No. BB-1585 USNA), the subject matter of which is hereby incorporated by reference in its entirety.

### Definitions

All References Cited Herein are Hereby Incorporated in their Entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

The term “invention” or “present invention” as used herein is not meant to be limiting to any one specific embodiment of the invention but applies generally to any and all embodiments of the invention as described in the claims and specification.

In the context of this disclosure, a number of terms and abbreviations are used. The following definitions are provided.

“Open reading frame” is abbreviated ORF.

“Polymerase chain reaction” is abbreviated PCR.

“American Type Culture Collection” is abbreviated ATCC.

“Polyunsaturated fatty acid(s)” is abbreviated PUFA(s).

“Triacylglycerols” are abbreviated TAGs.

The terms “down-regulate or down-regulation”, as used herein, refer to a reduction or decrease in the level of expression of a gene or polynucleotide.

The term “multizyme” refers to a single polypeptide having at least two independent and separable enzymatic activities. Preferably, the multizyme comprises a first enzymatic activity linked to a second enzymatic activity.

The term “fusion protein” is used interchangeably with the term “multizyme”. Thus, a “fusion protein” refers to a single polypeptide having at least two independent and separable enzymatic activities.

The term “fusion gene” refers to a polynucleotide or gene that encodes a multizyme. A fusion gene can be constructed by linking at least two DNA fragments, wherein each DNA fragment encodes for an independent and separate enzyme activity. An example of a fusion gene is described herein below in Example 4, in which the Hybrid1-HGLA Synthase fusion gene was constructed by linking the Euglena anabaena delta-9 elongase (EaD9Elo1; SEQ ID NO:39) and the Tetruetreptia pomquetensis CCMP1491 delta-8 desaturase (TpomD8; SEQ ID NO:26) using the Euglena gracilis DHA synthase 1 proline-rich linker (EgDHAsyn1 Link; SEQ ID NO:33). This and other examples of fusion genes are described in Applicants' Assignee's Application No. 12/061,738 (filed Apr. 3, 2008, which published Oct. 16, 2008; Attorney Docket No. BB-1585 USNA), the subject matter of which is hereby incorporated by reference in its entirety.

A “domain” or “functional domain” is a discrete, continuous part or subsequence of a polypeptide that can be associated with a function (e.g. enzymatic activity). As used herein, the term “domain” includes but is not limited to fatty acid biosynthetic enzymes and portions of fatty acid biosynthetic enzymes that retain enzymatic activity.

“DHA synthase” is an example of a multizyme. Specifically, a DHA synthase comprises a C20 elongase linked to a delta-4 desaturase using any of the linkers described herein.

The term “linker” refers to the bond or link between two or more polypeptides each having independent and separable enzymatic activities.

The preferred linker is a linker derived from a sequence of DHA synthase locus of a Euglenoid wherein the linker can be natural or synthetic. An example of a linker is shown in SEQ ID NO:34 (the EgDHAsyn1 proline-rich linker). Those skilled in the art will appreciate that it may be possible to make suitable amino acid substitutions in the linker such that it can function like a linker derived from a sequence of DHA synthase Euglenoid locus. Furthermore, it is possible to increase the distance between the polypeptides by using 2×, 3×, etc. the linker whether it is the sequence set forth in SEQ ID NO:34 or a comparable linker with one or more amino acid substitutions as described herein.

The term “Euglenoid” refers to Euglenophyceae which is a group of unicellular colorless or photosynthetic flagellates (“euglenoids”) found living in freshwater, marine, soil, and parasitic environments. The class is characterized by solitary unicells, wherein most are free-swimming and have two flagella (one of which may be nonemergent) arising from an anterior invagination known as a reservoir. Photosynthetic euglenoids contain one to many grass-green chloroplasts, which vary from minute disks to expanded plates or ribbons. Colorless euglenoids depend on osmotrophy or phagotrophy for nutrient assimilation. About 1000 species have been described and classified into about 40 genera and 6 orders. Examples of Euglenophyceae include, but are by no means limited to, the following genera: Euglena, Eutreptiella and Tetruetreptia.

The term “fatty acids” refers to long-chain aliphatic acids (alkanoic acids) of varying chain lengths, from about C₁₂ to C₂₂ (although both longer and shorter chain-length acids are known). The predominant chain lengths are between C₁₆ and C₂₂. Additional details concerning the differentiation between “saturated fatty acids” versus “unsaturated fatty acids”, “monounsaturated fatty acids” versus “polyunsaturated fatty acids” (or “PUFAs”), and “omega-6 fatty acids” (ω-6 or n-6) versus “omega-3 fatty acids” (omega-3 or n-3) are provided in U.S. Pat. No. 7,238,482.

Fatty acids are described herein by a simple notation system of “X:Y”, wherein X is the total number of carbon (C) atoms in the particular fatty acid and Y is the number of double bonds. The number following the fatty acid designation indicates the position of the double bond from the carboxyl end of the fatty acid with the “c” affix for the cis-configuration of the double bond (e.g., palmitic acid (16:0), stearic acid (18:0), oleic acid (18:1, 9c), petroselinic acid (18:1, 6c), LA (18:2, 9c,12c), GLA (18:3, 6c,9c,12c) and ALA (18:3, 9c,12c,15c)). Unless otherwise specified, 18:1, 18:2 and 18:3 refer to oleic, LA and ALA fatty acids, respectively. If not specifically written as otherwise, double bonds are assumed to be of the cis configuration. For instance, the double bonds in 18:2 (9,12) would be assumed to be in the cis configuration.

Nomenclature used to describe PUFAs in the present disclosure is shown below in Table 2. In the column titled “Shorthand Notation”, the omega-reference system is used to indicate the number of carbons, the number of double bonds and the position of the double bond closest to the omega carbon, counting from the omega carbon (which is numbered 1 for this purpose). The remainder of the table summarizes the common names of omega-3 and omega-6 fatty acids and their precursors, the abbreviations that will be used throughout the remainder of the specification, and each compounds' chemical name.

TABLE 2
Nomenclature of Polyunsaturated Fatty Acids and Precursors

| Common | | | Shorthand |
|---|---|---|---|
| Name | Abbreviation | Chemical Name | Notation |
| myristic | — | tetradecanoic | 14:0 |
| palmitic | PA or | hexadecanoic | 16:0 |
| | Palmitate | | |
| palmitoleic | — | 9-hexadecenoic | 16:1 |
| stearic | — | octadecanoic | 18:0 |
| oleic | — | cis-9-octadecenoic | 18:1 |
| linoleic | LA | cis-9,12-octadecadienoic | 18:2 ω-6 |
| gamma- | GLA | cis-6,9,12- | 18:3 ω-6 |
| linolenic | | octadecatrienoic | |
| eicosadienoic | EDA | cis-11,14-eicosadienoic | 20:2 ω-6 |
| dihomo- | DGLA or | cis-8,11,14-eicosatrienoic | 20:3 ω-6 |
| gamma- | HGLA (used | | |
| linolenic | interchangeably | | |
| | herein) | | |
| sciadonic | SCI | cis-5,11,14-eicosatrienoic | 20:3b ω-6 |
| arachidonic | ARA | cis-5,8,11,14- | 20:4 ω-6 |
| | | eicosatetraenoic | |
| alpha- | ALA | cis-9,12,15- | 18:3 ω-3 |
| linolenic | | octadecatrienoic | |
| stearidonic | STA | cis-6,9,12,15- | 18:4 ω-3 |
| | | octadecatetraenoic | |
| eicosatrienoic | ETrA or | cis-11,14,17- | 20:3 ω-3 |
| | ERA | eicosatrienoic | |
| eicosa- | ETA | cis-8,11,14,17- | 20:4 ω-3 |
| tetraenoic | | eicosatetraenoic | |
| juniperonic | JUP | cis-5,11,14,17- | 20:4b ω-3 |
| | | eicosatrienoic | |
| eicosa- | EPA | cis-5,8,11,14,17- | 20:5 ω-3 |
| pentaenoic | | eicosapentaenoic | |
| docosa- | DRA | cis-10,13,16- | 22:3 ω-3 |
| trienoic | | docosatrienoic | |
| docosa- | DTA | cis-7,10,13,16- | 22:4 ω-3 |
| tetraenoic | | docosatetraenoic | |
| docosa- | DPAn-6 | cis-4,7,10,13,16- | 22:5 ω-6 |
| pentaenoic | | docosapentaenoic | |
| docosa- | DPA | cis-7,10,13,16,19- | 22:5 ω-3 |
| pentaenoic | | docosapentaenoic | |
| docosa- | DHA | cis-4,7,10,13,16,19- | 22:6 ω-3 |
| hexaenoic | | docosahexaenoic | |

A metabolic, or biosynthetic, pathway, in a biochemical sense, can be regarded as a series of chemical reactions occurring within a cell, catalyzed by enzymes, to achieve either the formation of a metabolic product to be used or stored by the cell, or the initiation of another metabolic pathway (then called a flux generating step). Many of these pathways are elaborate, and involve a step by step modification of the initial substance to shape it into a product having the exact chemical structure desired.

The term “PUFA biosynthetic pathway” refers to a metabolic process that converts oleic acid to LA, EDA, GLA, DGLA, ARA, DTA, DPAn-6, ALA, STA, ETrA, ETA, EPA, DPA and DHA. This process is well described in the literature (e.g., see PCT Publication No. WO 2006/052870). Simplistically, this process involves elongation of the carbon chain through the addition of carbon atoms and desaturation of the molecule through the addition of double bonds, via a series of special desaturation and elongation enzymes (i.e., “PUFA biosynthetic pathway enzymes”) present in the endoplasmic reticulum membrane. More specifically, “PUFA biosynthetic pathway enzyme” refers to any of the following enzymes (and genes which encode said enzymes) associated with the biosynthesis of a PUFA, including: a delta-4 desaturase, a delta-5 desaturase, a delta-6 desaturase, a delta-12 desaturase, a delta-15 desaturase, a delta-17 desaturase, a delta-9 desaturase, a delta-8 desaturase, a delta-9 elongase, a C_{14/16} elongase, a C_{16/18} elongase, a C_{18/20} elongase, a C_{20/22} elongase, a DHA synthase and/or a multizyme of the instant invention.

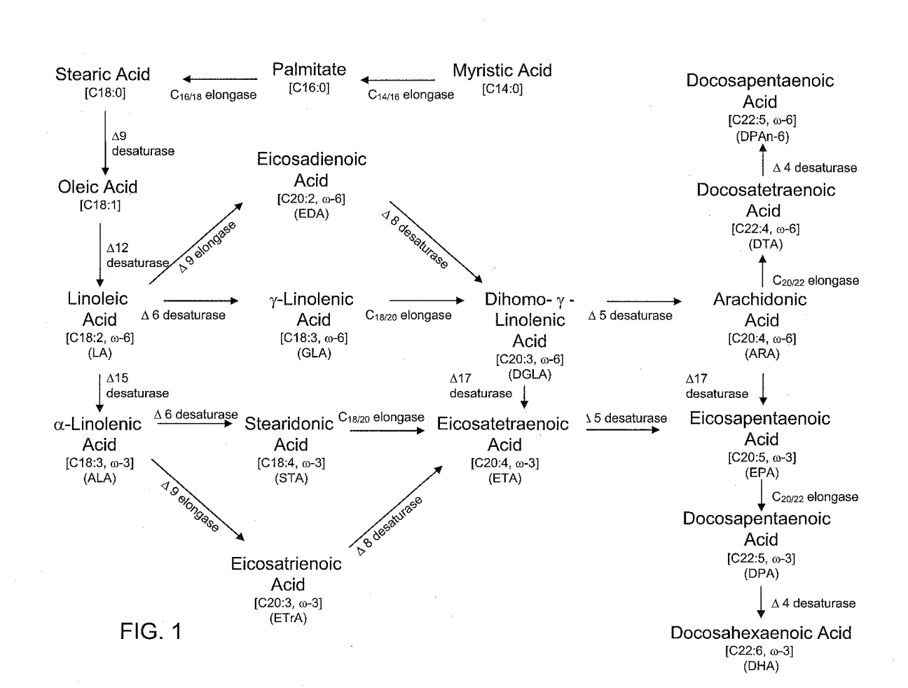

The term “omega-3/omega-6 fatty acid biosynthetic pathway” refers to a set of genes which, when expressed under the appropriate conditions encode enzymes that catalyze the production of either or both omega-3 and omega-6 fatty acids. Typically the genes involved in the omega-3/omega-6 fatty acid biosynthetic pathway encode PUFA biosynthetic pathway enzymes. A representative pathway is illustrated in FIG. 1, providing for the conversion of myristic acid through various intermediates to DHA, which demonstrates how both omega-3 and omega-6 fatty acids may be produced from a common source. The pathway is naturally divided into two portions where one portion will generate omega-3 fatty acids and the other portion, omega-6 fatty acids.

The term “functional” as used herein in context with the omega-3/omega-6 fatty acid biosynthetic pathway means that some (or all) of the genes in the pathway express active enzymes, resulting in in vivo catalysis or substrate conversion. It should be understood that “omega-3/omega-6 fatty acid biosynthetic pathway” or “functional omega-3/omega-6 fatty acid biosynthetic pathway” does not imply that all the PUFA biosynthetic pathway enzyme genes are required, as a number of fatty acid products will only require the expression of a subset of the genes of this pathway.

The term “delta-6 desaturase/delta-6 elongase pathway” refers to a PUFA biosynthetic pathway that minimally includes at least one delta-6 desaturase and at least one C_{18/20} elongase, thereby enabling biosynthesis of DGLA and/or ETA from LA and ALA, respectively, with GLA and/or STA as intermediate fatty acids. With expression of other desaturases and elongases, ARA, DTA, DPAn-6, EPA, DPA, and DHA may also be synthesized.

The term “delta-9 elongase/delta-8 desaturase pathway” refers to a PUFA biosynthetic pathway that minimally comprises at least one delta-9 elongase and at least one delta-8 desaturase, thereby enabling biosynthesis of DGLA and/or ETA from LA and ALA, respectively, with EDA and/or ETrA as intermediate fatty acids With expression of other desaturases and elongases, ARA, DTA, DPAn-6, EPA, DPA and DHA may also be synthesized. This pathway may be advantageous as the biosynthesis of GLA and/or STA is excluded.

The term “intermediate fatty acid” refers to any fatty acid produced in a fatty acid metabolic pathway that can be further converted to an intended product fatty acid in this pathway by the action of other metabolic pathway enzymes. For instance, when EPA is produced using the delta-9 elongase/delta-8 desaturase pathway, EDA, ETrA, DGLA, ETA and ARA can be produced and are considered “intermediate fatty acids” since these fatty acids can be further converted to EPA via action of other metabolic pathway enzymes.

The term “by-product fatty acid” refers to any fatty acid produced in a fatty acid metabolic pathway that is not the intended fatty acid product of the pathway nor an “intermediate fatty acid” of the pathway. For instance, when EPA is produced using the delta-9 elongase/delta-8 desaturase pathway, sciadonic acid (SCI) and juniperonic acid (JUP) also can be produced by the action of a delta-5 desaturase on either EDA or ETrA, respectively. They are considered to be “by-product fatty acids” since neither can be further converted to EPA by the action of other metabolic pathway enzymes.

The terms “triacylglycerol”, “oil” and “TAGs” refer to neutral lipids composed of three fatty acyl residues esterified to a glycerol molecule (and such terms will be used interchangeably throughout the present disclosure herein). Such oils can contain long-chain PUFAs, as well as shorter saturated and unsaturated fatty acids and longer chain saturated fatty acids. Thus, “oil biosynthesis” generically refers to the synthesis of TAGs in the cell.

“Percent (%) PUFAs in the total lipid and oil fractions” refers to the percent of PUFAs relative to the total fatty acids in those fractions. The term “total lipid fraction” or “lipid fraction” both refer to the sum of all lipids (i.e., neutral and polar) within an oleaginous organism, thus including those lipids that are located in the phosphatidylcholine (PC) fraction, phosphatidylethanolamine (PE) fraction and triacylglycerol (TAG or oil) fraction. However, the terms “lipid” and “oil” will be used interchangeably throughout the specification.

The terms “conversion efficiency” and “percent substrate conversion” refer to the efficiency by which a particular enzyme (e.g., a desaturase) can convert substrate to product. The conversion efficiency is measured according to the following formula: ([product]/[substrate+product])*100, where ‘product’ includes the immediate product and all products in the pathway derived from it.

“Desaturase” is a polypeptide that can desaturate, i.e., introduce a double bond, in one or more fatty acids to produce a fatty acid or precursor of interest. Despite use of the omega-reference system throughout the specification to refer to specific fatty acids, it is more convenient to indicate the activity of a desaturase by counting from the carboxyl end of the substrate using the delta-system. For example, delta-8 desaturases will desaturate a fatty acid between the eighth and ninth carbon atom numbered from the carboxyl-terminal end of the molecule and can, for example, catalyze the conversion of EDA to DGLA and/or ETrA to ETA. Other useful fatty acid desaturases include, for example: (1) delta-5 desaturases that catalyze the conversion of DGLA to ARA and/or ETA to EPA; (2) delta-6 desaturases that catalyze the conversion of LA to GLA and/or ALA to STA; (3) delta-4 desaturases that catalyze the conversion of DPA to DHA and/or DTA to DPAn-6; (4) delta-12 desaturases that catalyze the conversion of oleic acid to LA; (5) delta-15 desaturases that catalyze the conversion of LA to ALA and/or GLA to STA; (6) delta-17 desaturases that catalyze the conversion of ARA to EPA and/or DGLA to ETA; and (7) delta-9 desaturases that catalyze the conversion of palmitic acid to palmitoleic acid (16:1) and/or stearic acid to oleic acid (18:1). In the art, delta-15 and delta-17 desaturases are also occasionally referred to as “omega-3 desaturases”, “w-3 desaturases”, and/or “n-3 desaturases”, based on their ability to convert omega-6 fatty acids into their omega-3 counterparts (e.g., conversion of LA into ALA and ARA into EPA, respectively). It can be desirable to empirically determine the specificity of a particular fatty acid desaturase by transforming a suitable host with the gene for the fatty acid desaturase and determining its effect on the fatty acid profile of the host.

The term “delta-4 desaturase” refers to an enzyme that will desaturate a fatty acid between the fourth and fifth carbon atom numbered from the carboxyl-terminal end of the molecule and that can, for example, catalyze the conversion of DPA to DHA and/or DTA to DPAn-6. For the purposes herein, the term “EgDHAsyn1” refers to a DHA synthase enzyme (SEQ ID N0:5) isolated from Euglena gracilis, encoded by SEQ ID NO:4 herein.

The term “EgDHAsyn2” refers to a DHA synthase enzyme (SEQ ID NO:8) isolated from Euglena gracilis, encoded by SEQ ID NO:7 herein. The term “EaDHAsyn1” refers to a DHA synthase enzyme (SEQ ID NO:18) isolated from Euglena anabaena, encoded by SEQ ID NO:17 herein. The term “EaDHAsyn2” refers to a DHA synthase enzyme (SEQ ID NO:20) isolated from Euglena anabaena, encoded by SEQ ID NO:19 herein. The term “EaDHAsyn3” refers to a DHA synthase enzyme (SEQ ID NO:22) isolated from Euglena anabaena, encoded by SEQ ID NO:21 herein. The term “EaDHAsyn4” refers to an enzyme (SEQ ID NO:24) isolated from Euglena anabaena, encoded by SEQ ID NO:23 herein.

The term “elongase system” refers to a suite of four enzymes that are responsible for elongation of a fatty acid carbon chain to produce a fatty acid that is two carbons longer than the fatty acid substrate that the elongase system acts upon. More specifically, the process of elongation occurs in association with fatty acid synthase, whereby CoA is the acyl carrier (Lassner et al., Plant Cell 8:281-292 (1996)). In the first step, which has been found to be both substrate-specific and also rate-limiting, malonyl-CoA is condensed with a long-chain acyl-CoA to yield carbon dioxide (CO₂) and a β-ketoacyl-CoA (where the acyl moiety has been elongated by two carbon atoms). Subsequent reactions include reduction to β-hydroxyacyl-CoA, dehydration to an enoyl-CoA and a second reduction to yield the elongated acyl-CoA. Examples of reactions catalyzed by elongase systems are the conversion of GLA to DGLA, STA to ETA, LA to EDA, ALA to ETrA and EPA to DPA.

For the purposes herein, an enzyme catalyzing the first condensation reaction (i.e., conversion of malonyl-CoA and long-chain acyl-CoA to β-ketoacyl-CoA) will be referred to generically as an “elongase”. In general, the substrate selectivity of elongases is somewhat broad but segregated by both chain length and the degree of unsaturation. Accordingly, elongases can have different specificities. For example, a C_{14/16} elongase will utilize a C₁₄ substrate (e.g., myristic acid); a C_{16/18} elongase will utilize a C₁₆ substrate (e.g., palmitate); a C_{18/20} elongase will utilize a C₁₈ substrate (e.g., GLA, STA); and a C_{20/22} elongase will utilize a C₂₀ substrate (e.g., ARA, EPA). Similarly, a “delta-9 elongase” is able to catalyze the conversion of LA to EDA and/or ALA to ETrA.

It is should be noted that some elongases have broad specificity and thus a single enzyme may be capable of catalyzing several elongase reactions. Thus, for example, a delta-9 elongase may also act as a C_{16/18} elongase, C_{18/20} elongase and/or C_{20/22} elongase and may have alternate, but not preferred, specificities for delta-5 and delta-6 fatty acids such as EPA and/or GLA, respectively.

The term “C20 elongase” as used herein refers to an enzyme which utilizes a C20 substrate such as EPA or ARA, for example. The term “C20/delta-5 elongase” refers to an enzyme that utilizes a C20 substrate with a delta-5 double bond.

Similarly for the purposes herein, the term “EgD9elo” or “EgD9e” refers to a delta-9 elongase isolated from Euglena gracilis (see SEQ ID NO:25; also see U.S. application Ser. No. 11/601,563 (filed Nov. 16, 2006, which published as US-2007-0118929-A1 on May 24, 2007)).

As used herein, “nucleic acid” means a polynucleotide and includes a single or double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” or “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric genes to produce the desired phenotype in a transformed plant. Chimeric genes can be designed for use in suppression by linking a nucleic acid fragment or subfragment thereof, whether or not it encodes an active enzyme, in the sense or antisense orientation relative to a plant promoter sequence.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential in the structure, the stability, or the activity of a protein. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. Moreover, the skilled artisan recognizes that substantially similar nucleic acid sequences encompassed by this invention are also defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth et al., Anal. Biochem. 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995). Hybridization and/or wash conditions can be applied for at least 10, 30, 60, 90, 120, or 240 minutes.

“Sequence identity” or “identity” in the context of nucleic acid or polypeptide sequences refers to the nucleic acid bases or amino acid residues in two sequences that are the same when aligned for maximum correspondence over a specified comparison window.

Thus, “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, CABIOS. 5:151-153 (1989); Higgins, D. G. et al., Comput. Appl. Biosci. 8:189-191 (1992)) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, supra; Higgins, D. G. et al., supra) and found in the MegAlign™ v6.1 program of the LASERGENE bioinformatics computing suite (DNASTAR Inc., Madison, Wis.). Default parameters for multiple alignment correspond to GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB. After alignment of the sequences using the Clustal W program, it is possible to obtain a “percent identity” by viewing the “sequence distances” table in the same program.

“BLASTN method of alignment” is an algorithm provided by the National Center for Biotechnology Information (NCBI) to compare nucleotide sequences using default parameters.

It is well understood by one skilled in the art that many levels of sequence identity are useful in identifying polypeptides and/or nucleotides, from other species, wherein such polypeptides and/or nucleotides have the same or similar function or activity. Useful examples of percent identities include, but are not limited to, 50%, 55%, 60%, 65%, 70%, 75%, 80%, 85%, 90%, or 95%, or any integer percentage from 50% to 100%. Indeed, any integer amino acid identity and/or nucleotide identity from 50% to 100% may be useful in describing the present invention, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Also, of interest is any full-length or partial complement of a nucleotide sequence comprised withing an isolated nucleotide fragment.

“Gene” refers to a nucleic acid fragment that expresses a specific protein and can include either the coding region alone or the coding region in addition to the regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genome” as it applies to plant cells encompasses not only chromosomal DNA found within the nucleus, but organelle DNA found within subcellular components (e.g., mitochondrial, plastid) of the cell.

A “codon-optimized gene” is a gene having its frequency of codon usage designed to mimic the frequency of preferred codon usage of the host cell.

An “allele” is one of several alternative forms of a gene occupying a given locus on a chromosome. When all the alleles present at a given locus on a chromosome are the same that plant is homozygous at that locus. If the alleles present at a given locus on a chromosome differ that plant is heterozygous at that locus.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”. New promoters of various types useful in plant cells are constantly being discovered; numerous examples may be found in the compilation by Okamuro, J. K., and Goldberg, R. B. Biochemistry of Plants 15:1-82 (1989).

“Translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Mol. Biotechnol. 3:225-236 (1995)).

“3′ non-coding sequences”, “transcription terminator” or “termination sequences” refer to DNA sequences located downstream of a coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. Plant Cell 1:671-680 (1989).

“RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks or reduces the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory: Cold Spring Harbor, N.Y. (1989). Transformation methods are well known to those skilled in the art and are described infra.

“PCR” or “polymerase chain reaction” is a technique for the synthesis of large quantities of specific DNA segments and consists of a series of repetitive cycles (Perkin Elmer Cetus Instruments, Norwalk, Conn.). Typically, the double-stranded DNA is heat denatured, the two primers complementary to the 3′ boundaries of the target segment are annealed at low temperature and then extended at an intermediate temperature. One set of these three consecutive steps is referred to as a “cycle”.

The term “recombinant” refers to an artificial combination of two otherwise separated segments of sequence, e.g., by chemical synthesis or by the manipulation of isolated segments of nucleic acids by genetic engineering techniques.

A “plasmid” or “vector” is an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing an expression cassette(s) into a cell. “Expression cassette” refers to a fragment of DNA containing a foreign gene and having elements in addition to the foreign gene that allow for enhanced expression of that gene in a foreign host. “Transformation cassette” refers to a fragment of DNA containing a foreign gene and having elements in addition to the foreign gene that facilitate transformation of a particular host cell.

The terms “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The term “expression”, as used herein, refers to the production of a functional end-product (e.g., an mRNA or a protein [either precursor or mature]).

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms.

As used herein, “transgenic” refers to a plant or a cell which comprises within its genome a heterologous polynucleotide. Preferably, the heterologous polynucleotide is stably integrated within the genome such that the polynucleotide is passed on to successive generations. The heterologous polynucleotide may be integrated into the genome alone or as part of an expression construct. Transgenic is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020). Co-suppression constructs in plants previously have been designed by focusing on overexpression of a nucleic acid sequence having homology to an endogenous mRNA, in the sense orientation, which results in the reduction of all RNA having homology to the overexpressed sequence (Vaucheret et al., Plant J. 16:651-659 (1998); Gura, Nature 404:804-808 (2000)). The overall efficiency of this phenomenon is low, and the extent of the RNA reduction is widely variable. More recent work has described the use of “hairpin” structures that incorporate all, or part, of an mRNA encoding sequence in a complementary orientation that results in a potential “stem-loop” structure for the expressed RNA (PCT Publication No. WO 99/53050; PCT Publication No. WO 02/00904). This increases the frequency of co-suppression in the recovered transgenic plants. Another variation describes the use of plant viral sequences to direct the suppression, or “silencing”, of proximal mRNA encoding sequences (PCT Publication No. WO 98/36083). Both of these co-suppressing phenomena have not been elucidated mechanistically, although genetic evidence has begun to unravel this complex situation (Elmayan et al., Plant Cell 10:1747-1757 (1998)).

The term “oleaginous” refers to those organisms that tend to store their energy source in the form of lipid (Weete, In: Fungal Lipid Biochemistry, 2^{nd} Ed., Plenum, 1980). A class of plants identified as oleaginous are commonly referred to as “oilseed” plants. Examples of oilseed plants include, but are not limited to: soybean (Glycine and Soja sp.), flax (Linum sp.), rapeseed (Brassica sp.), maize, cotton, safflower (Carthamus sp.) and sunflower (Helianthus sp.).

Within oleaginous microorganisms the cellular oil or TAG content generally follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol. 57:419-25 (1991)). The term “oleaginous yeast” refers to those microorganisms classified as yeasts that make oil. It is not uncommon for oleaginous microorganisms to accumulate in excess of about 25% of their dry cell weight as oil. Examples of oleaginous yeast include, but are no means limited to, the following genera: Yarrowia, Candida, Rhodotorula, Rhodosporidium, Cryptococcus, Trichosporon and Lipomyces.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores.

“Progeny” comprises any subsequent generation of a plant.

In one embodiment, the present invention concerns a multizyme comprising a single polypeptide having at least two independent and separable activities wherein the independent and separable activities are joined together using a linker derived from a sequence of a DHA synthase locus of a Euglenoid wherein said linker can be natural or synthetic provided that the multizyme does not comprise (i) a fatty acid desaturase linked to a fatty acid elongase or (ii) a fatty acid desaturase linked to another fatty acid desaturase.

Examples of suitable activities that can be linked together include, but are not limited to nitrite reductase and glutamine synthetase; sucrose:sucrose fructosyltransferase (SST) and fructan:fructan fructosyltransrerase (FFT); a prokaryotic prephenate dehydrogenase or bifunctional chorismate mutase/prephenate dehydrogenase or a plant prephenate dehydrogenase (PDH) linked to a HPPD (4-hydroxyphenylpyruvate dioxygenase) activity; 1-Hydroxy-2-methyl-2-(E)-butenyl 4-diphosphate reductase (HMBDP reductase, also referred to as IDS; EC 1.17.1.2) linked to an adenosine phosphate isopentenyltransferase (IPT; EC 2.5.1.27); a herbicide resistant plant acetolactate synthase subunit linked to another herbicide resistant plant acetolactate synthase subunit; a soluble nitrate reductase (NR) to a nitrate transporter (NT); or an acyl-ACP thioesterase linked to a soluble beta-ketopacylsynthase II.

Examples of suitable transferases include but are not limited to acyl transferases such as glycerol-3-phosphate O-acyltransferase (also called glycerol-phosphate acyl transferase or glycerol-3-phosphate acyl transferase; GPAT), 2-acylglycerol O-acyltransferase, 1-acylglycerol-3-phosphate O-acyltransferase (also called 1-acylglycerol-phosphate acyltransferase or lyso-phosphatidic acid acyltransferase; AGPAT or LPAAT or LPAT), 2-acylglycerol-3-phosphate O-acyltransferase, 1-acylglycerophosphocholine O-acyltransferase (also called lyso-lecithin acyltransferase or lyso-phosphatidylcholine acyltransferase; AGPCAT or LLAT or LPCAT), 2-acylglycerophosphocholine O-acyltransferase, diacylglycerol O-acyltransferase (also called diglyceride acyltransferase; DAGAT or DGAT) and phospholipid:diacylglycerol acyltransferase (PDAT).

An example of a suitable acyl CoA synthetase includes but is not limited to long-chain-fatty-acid-CoA ligase (also called acyl-activating enzyme or acyl-CoA synthetase).

An example of a suitable thioesterase includes but is not limited to oleoyl-[acyl-carrier-protein]hydrolase (also called acyl-[acyl-carrier-protein]hydrolase, acyl-ACP-hydrolase or acyl-ACP-thioesterase).

The link used to form the multizyme should be derived from a sequence of a DHA synthase locus of a Euglenoid. The linker can be natural or synthetic provided that the multizyme does not comprise (i) a fatty acid desaturase linked to a fatty acid elongase; or (ii) a fatty acid desaturase linked to another fatty acid desaturase.

It is possible that amino acid substitutions could be made while retaining the ability to form a multizyme as if it was a linker derived from a sequence of a DHA synthase locus of a Euglenoid. Furthermore, the Euglenoid DHA synthase linker or substituted linker can be used at least once or more than once to extend the linkage between at least two polypeptides, if so needed.

As is discussed above, multizymes were discovered when a unique DHA synthase gene that was isolated from Euglena and Euglenoid homologs. This DHA synthase was found to be a fusion of a delta-4 desaturase and a C20 condensing enzyme (an Elo-type elongase). This was the first time that two enzymes with very different activities/functions were found to be fused in this way. Data described herein confirm that linking of the two domains within each synthase results in increased efficiency or flux, as compared to efficiency or flux observed when the enzymatic domains exist as independent entities, i.e., not linked together in a multizyme.

Nucleotide sequences encoding DHA synthases have been isolated from Euglena gracilis and Euglena anabaena, as summarized below in Table 3.

TABLE 3
Summary Of Euglena DHA Synthases

| DHA Synthase | | Nucleotide | Amino Acid |
|---|---|---|---|
| Designation | Organism | SEQ ID NO | SEQ ID NO |
| EgDHAsyn1 | E. gracilis | 4 | 5 |
| EgDHAsyn1* | E. gracilis | 37 | 5 |
| EgDHAsyn2 | E. gracilis | 7 | 8 |
| EaDHAsyn1 | E. anabaena | 17 | 18 |
| EaDHAsyn2 | E. anabaena | 19 | 20 |
| EaDHAsyn3 | E. anabaena | 21 | 22 |
| EaDHAsyn4 | E. anabaena | 23 | 24 |

Those skilled in the art will appreciate that EgDHAsyn1, EgDHAsyn2, EaDHAsyn1, EaDHAsyn2, EaDHAsyn3 and EaDHAsyn34 DHA synthase sequences can be codon-optimized for expression in a particular host organism. As is well known in the art, this can be a useful means to further optimize the expression of the enzyme in the alternate host, since use of host-preferred codons can substantially enhance the expression of the foreign gene encoding the polypeptide. EgDHAsyn1, for example, can be codon-optimized for expression in Yarrowia lipolytica, thereby yielding EgDHAsyn1S (as taught in U.S. Pat. No. 7,238,482 and U.S. Pat. No. 7,125,672).

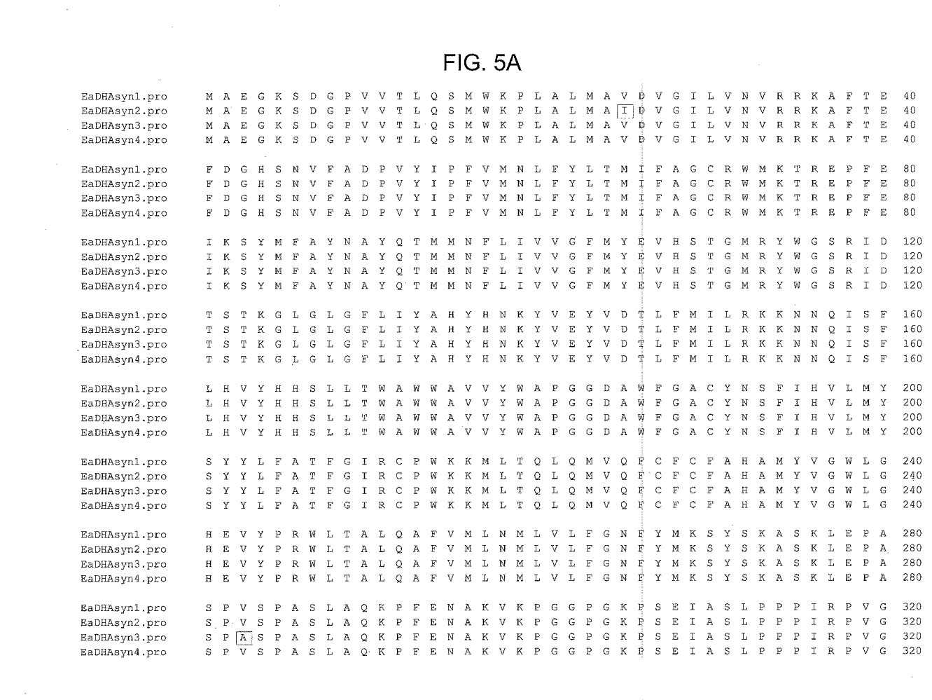

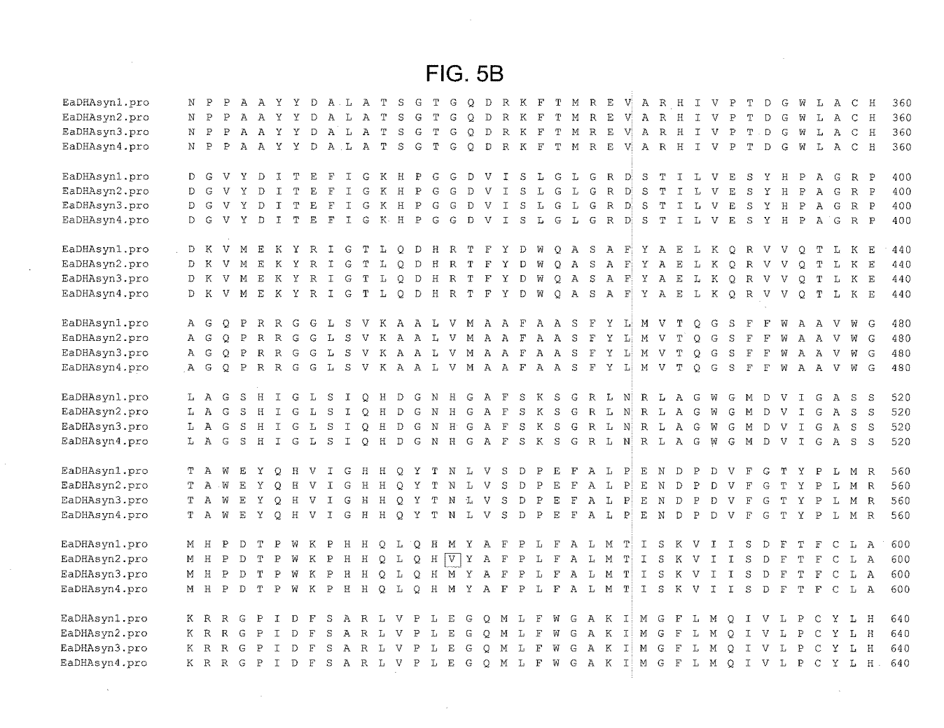

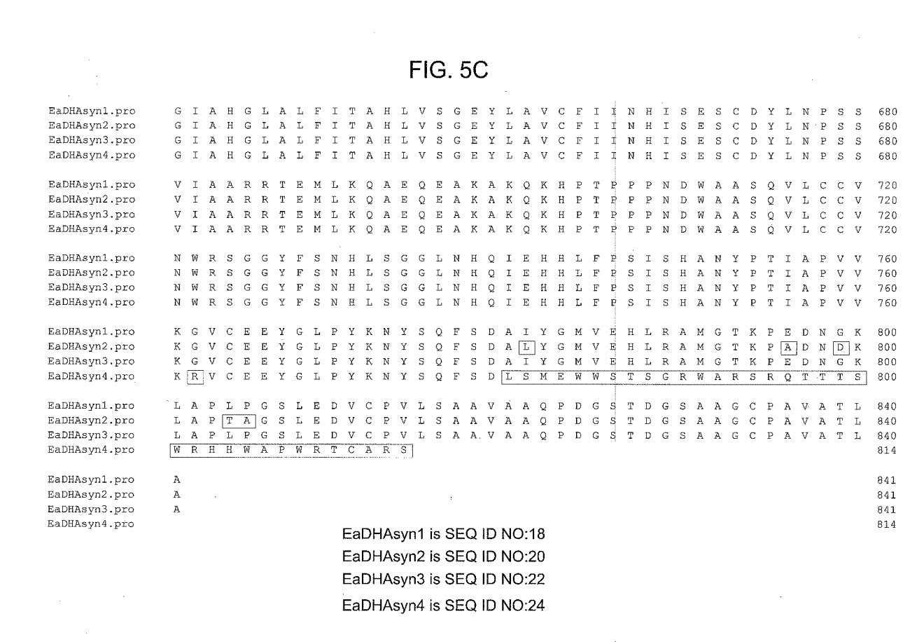

An alignment of the amino acid sequences for EaDHAsyn1 (SEQ ID NO:18), EaDHAsyn2 (SEQ ID NO:20), EaDHAsyn3 (SEQ ID NO:22) and EaDHAsyn4 (SEQ ID NO:24) using the Clustal W method is shown in FIGS. 5A, 5B and 5C.

When compared to the amino acid sequence of EgDHAsyn1 (SEQ ID NO:5) using BLASTP, the amino acid sequences of EaDHAsyn1 (SEQ ID NO:18), EaDHAsyn2 (SEQ ID NO:20), EaDHAsyn3 (SEQ ID NO:22) and EaDHAsyn4 (SEQ ID NO:24) were 70% (558/791), 70% (558/791), 70% (559/791) and 70% (548/775) identical, respectively.

As was the case for EgDHAsyn1 (SEQ ID NO:5) and EgDHAsyn2 (SEQ ID NO:8), all four EaDHAsyn sequences have a proline-rich linker region (from approximately P300 to T332 based on numbering for EaDHAsyn1). The linker appears to be slightly longer than that for EgDHAsyn1 (SEQ ID NO:5) or EgDHAsyn2 (SEQ ID NO:8). All four EaDHAsyn sequences also lack the NG repeat motif found upstream of the proline-rich motif of EgDHAsyn1 and EgDHAsyn2; but, this region, as was the case for EgDHAsyn1 and EgDHAsyn2, is also slightly proline-rich in all four EaDHAsyn sequences and may play a role in the linker function.

The nucleotide and amino acid sequences for the C20 elongase domains of EaDHAsyn1, EaDHAsyn2, EaDHAsyn3 and EaDHAsyn4 are described in Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety.

The nucleotide and amino acid sequence for the proline-rich linker of EaDHAsyn1 is set forth in SEQ ID NO:49 and SEQ ID NO:50, respectively. The nucleotide and amino acid sequences for the proline-rich linkers of EaDHAsyn2, EaDHAsyn3 and EaDHAsyn4 are identical to that for EaDHAsyn1.

One skilled in the art would be able to use the teachings herein to create various other codon-optimized DHA synthase proteins suitable for optimal expression in alternate hosts, based on the wildtype EgDHAsyn1, EgDHAsyn2, EaDHAsyn1, EaDHAsyn2 and/or EaDHAsyn3 sequences described above in Table 3.

It may be desirable to modify a portion of the codons encoding EgDHAsyn1, EgDHAsyn2, EaDHAsyn1, EaDHAsyn2 and/or EaDHAsyn3 to enhance expression of the gene in a host organism including, but not limited to, a plant or plant part.

Any of the DHA synthase sequences described herein (i.e., EgDHAsyn1, EgDHAsyn2, EaDHAsyn1, EaDHAsyn2 and EaDHAsyn3) or portions thereof may be used to search for DHA synthase homologs in the same or other bacterial, algal, fungal, euglenoid or plant species using sequence analysis software. In general, such computer software matches similar sequences by assigning degrees of homology to various substitutions, deletions, and other modifications.

Alternatively, any of the instant DHA synthase sequences or portions thereof may also be employed as hybridization reagents for the identification of DHA synthase homologs. The basic components of a nucleic acid hybridization test include a probe, a sample suspected of containing the gene or gene fragment of interest and a specific hybridization method. Probes of the present invention are typically single-stranded nucleic acid sequences that are complementary to the nucleic acid sequences to be detected. Probes are “hybridizable” to the nucleic acid sequence to be detected. Although the probe length can vary from 5 bases to tens of thousands of bases, typically a probe length of about 15 bases to about 30 bases is suitable. Only part of the probe molecule needs to be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions that will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally, a chaotropic agent may be added (e.g., guanidinium chloride, guanidinium thiocyanate, sodium thiocyanate, lithium tetrachloroacetate, sodium perchlorate, rubidium tetrachloroacetate, potassium iodide, cesium trifluoroacetate). If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kdal), polyvinylpyrrolidone (about 250-500 kdal), and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents that include a variety of polar water-soluble or swellable agents (e.g., polyethylene glycol), anionic polymers (e.g., polyacrylate or polymethylacrylate) and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it an immobilized nucleic acid probe that is unlabeled and complementary to one portion of the sequence.

Any of the DHA synthase nucleic acid fragments described herein (or any homologs identified thereof) may be used to isolate genes encoding homologous proteins from the same or other bacterial, algal, fungal, euglenoid or plant species. Isolation of homologous genes using sequence-dependent protocols is well known in the art. Examples of sequence-dependent protocols include, but are not limited to: (1) methods of nucleic acid hybridization; (2) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor et al., Proc. Acad. Sci. USA 82:1074 (1985); or strand displacement amplification (SDA), Walker et al., Proc. Natl. Acad. Sci. U.S.A., 89:392 (1992)]; and (3) methods of library construction and screening by complementation.

For example, genes encoding similar proteins or polypeptides to a multizyme or an individual domain thereof (such as the DHA synthases) described herein, could be isolated directly by using all or a portion of the instant nucleic acid fragments as DNA hybridization probes to screen libraries from e.g., any desired yeast or fungus using methodology well known to those skilled in the art.

Specific oligonucleotide probes based upon the instant nucleic acid sequences can be designed and synthesized by methods known in the art (Maniatis, supra). Moreover, the entire sequences can be used directly to synthesize DNA probes by methods known to the skilled artisan (e.g., random primers DNA labeling, nick translation or end-labeling techniques), or RNA probes using available in vitro transcription systems. In addition, specific primers can be designed and used to amplify a part of (or full-length of) the instant sequences. The resulting amplification products can be labeled directly during amplification reactions or labeled after amplification reactions, and used as probes to isolate full-length DNA fragments under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotide as specific hybridization probes in the Diagnosis of Genetic Disorders”, in Human Genetic Diseases: A Practical Approach, K. E. Davis Ed., (1986) pp 33-50, IRL: Herndon, V A; and Rychlik, W., In Methods in Molecular Biology, White, B. A. Ed., (1993) Vol. 15, pp 31-39, PCR Protocols: Current Methods and Applications. Humania: Totowa, N.J.).

Generally two short segments of the instant sequences may be used in PCR protocols to amplify longer nucleic acid fragments encoding homologous genes from DNA or RNA. PCR may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding eukaryotic genes.

Alternatively, the second primer sequence may be based upon sequences derived from the cloning vector. For example, the skilled artisan can follow the RACE protocol (Frohman et al., Proc. Natl Acad. Sci. U.S.A., 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (Gibco/BRL, Gaithersburg, Md.), specific 3′ or 5′ cDNA fragments can be isolated (Ohara et al., Proc. Natl Acad. Sci. U.S.A., 86:5673 (1989); Loh et al., Science 243:217 (1989)).

Any of the multizymes, DHA synthases, or individual domains described herein may be modified. As is well known to those skilled in the art, in vitro mutagenesis and selection, chemical mutagenesis, “gene shuffling” methods or other means can be employed to obtain mutations of naturally occurring genes. Alternatively, multizymes may be synthesized by domain swapping, wherein a functional domain from any enzyme may be exchanged with or added to a functional domain in an alternate enzyme to thereby result in a novel protein.

As was noted above, multizymes of the invention can be used in a variety of applications that extend beyond the biosynthesis of long chain polyunsaturated fatty acids.

A multizyme of the invention can be used to increase total flux of all combined reactions of the multizyme when compared with the total flux of the combined reactions when each reaction is produced separately when not linked together as the multizyme. A metabolic pathway can be constructed as a single multizyme and the total flux through the multizyme pathway will be greater than the individual reactions of the pathway expressed together but as separate activities. Such a multizyme pathway, with two or more of the activities of the pathway linked in a multizyme, can be engineered into a transgenic plant to increase the accumulation of a novel product when compared with a pathway engineered from individually expressed activities. A fusion of nitrite reductase and glutamine synthetase enzymes can result in an increased flux of nitrite to glutamate and ultimately lead to increased amino acid production. The importance of the nitrogen assimilating enzymes is well know in the art (see for example Heldt H. 1996. Plant Biochemistry and Molecular Biology. Oxforn University press. ISBN 019850180, chapter 10, page 247-277).

Another example can be found in fusing a sucrose:sucrose fructosyltransferase (SST) with a fructan:fructan fructosyltransrerase (FFT) which can result in an increased flux of sucrose (the substrate of SST through the SST-FFT pathway leading to increased inulin production. SST and FFT are the enzymes responsible for inulin biosynthesiused using the Euglenoid linker are more efficient at moving substrate through both reactions, and thus increasing the flux through the reactions, than enzymes expressed together as separate proteins.

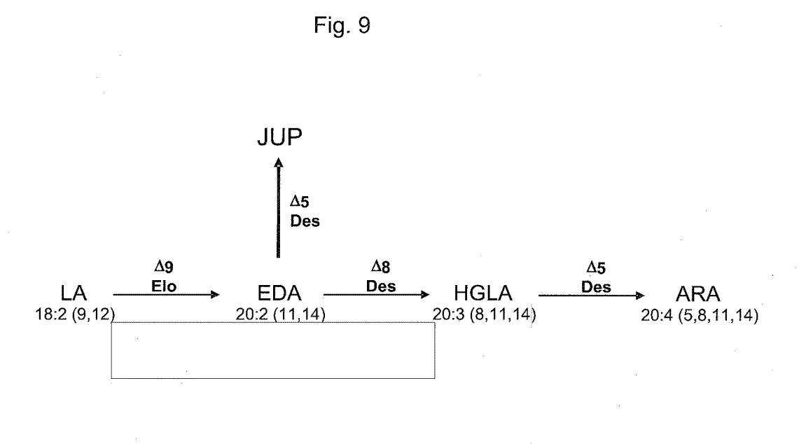

Linking a delta-9 elongase together with a delta-8 desaturase using a linker to form a multizyme (DGLA and/or ETA synthase), for example, could result in increased flux through these steps leading to reduced availability of the EDA/ERA intermediate fatty acids to delta-5 desaturase and thus reduced concentrations of SCI and JUP (FIG. 9). Evidence for an increased flux of all combined reactions using a multizyme involved in fatty acid metabolism is found in Example 61 of Applicants' Assignee's U.S. application Ser. No. 12/061,738 (filed Apr. 3, 2008, which published Oct. 16, 2008; Attorney Docket No. BB-1585 USNA), (triple fusion for ARA biosynthesis).

A multizyme of the invention can be used in a reaction sequence to lower an amount of at least one intermediate produced by the multizyme when compared to the amount of substantially the same intermediate produced by each individual enzymatic activity in a reaction sequence when not linked together as a multizyme.

A metabolic pathway can be constructed as a single multizyme or with two or more of the activities of the pathway linked in a multizyme and the intermediates and by-products (non target products) of the pathway will be eliminated or reduced when compared with a pathway engineered from individually expressed activities. For instance, linking a HMBDP reductase with an IPT in a multizyme using the Euglemoid linker, may lead to a decrease in the intermediates DMAPP (dimethylallyl diphosphate) and IPP (isopentenyl diphosphate) during production of iPRMP (isopentenyladenine riboside monophosphate).

Similarly, linking an SST and an FFT may reduce the buildup of short chain inulin polymers (product of SST).The specific genes included within a particular expression cassette will depend on the host cell, the availability of substrate and the desired end product(s).

A multizyme of the invention can be used in a reaction to reduce the extent of any feedback inhibition of at least one enzymatic activity of the multizyme when compared to the amount of feedback inhibition of a corresponding enzymatic activity that occurs when said individual enzymatic activity is not part of a multizyme. A multizyme can be used to engineer a pathway that is normally regulated by the feedback inhibition of a pathway product on one the activites in the pathway. If the regulated acticvitiy is part of a multizyme it will be resistant to inhibition by the pathway intermediate or product and thus the pathway will overproduce a final product.

A multizyme of the invention can be used in a reaction to protect an activity of the multizyme from competitive inhibition by a substrate other than that produced by a preceding enzymatic activity of the multizyme. Many enzyme inhibitors, such as herbicides and antibiotics, are enzyme substrate analogs that comptetively inhibit the enzyme. An enzyme resistant to competitive inhibition, and thus herbicide or antibiotic resistance, can be engineered by linking it in a multizyme to another activity that supplies the appropriate substrate. Thus a multizyme of the invention can be used such that at least two enzymatic activities which when linked together have resistance to an herbicide inhibitor of at least one of the individual activities. For example, the multizyme can comprise a 4-hydroxyphenyl pyruvate dioxygenase linked to an activity that produces 4-hydroxyphenylpyruvate or it can comprises a 4-hydroxyphenyl pyruvate dioxygenase linked to a prephenate dehydrogenase or a bifunctional chorismate PDH. A HPPD/PDH mutase/multizyme can be created by joining a HPPD with a prokaryotic or plant PDH or with a prokaryotic bifunctional chorismate mutase/PDH together using a linker derived from a sequence of a DHA synthase locus of a Euglenoid. Alternatively, a HPPD/tyrosine aminotransferase multizyme can be created by joiing a HPPD and a tyrosine aminotransferase. Fusing HPPD with a prokaryotic or plant PDH or with a prokaryotic bifunctional chorismate mutase/PDH can protect the HPPD from competitive-substrate inhibition and create a herbicide-resistant HPPD activity.

A multizyme of the invention can be used such that a product of a reaction of the multizyme is substantially used in a subsequent reaction of the multizyme.

Linking a HMBDP reductase with an IPT in a multizyme using a linker derived from a sequence of a DHA synthase locus of a Euglenoid can channel carbon into cytokinin by maximizing DMAPP production and reducing IPP formation. Such a multizyme can also serve to divert more DMAPP into the CK pathway rather than to isoprenenoids.

In a seventh embodiment, the multizyme of the invention can be used such that at least one independent and separable activity of the multizyme is prevented from associating with polypeptide complexes other than the multizyme of which it is a part. An activity may be regulated by its association with another polypeptide, or it may form homo- or hetero-dimers with similar acticities and this may regulate enzyme activity. Association of a modified protein with a native homolog of the protein may reduice the efficacy of the modification and this may be prevented by engineering multizyme homodimers that prevent the formation of heterodimers. For example a herbicide resistant acetolactate synthase (HRA) acivitiy may be linked to a second HRA activity to prevent association of the HRA subunit with a non-herbicide resistant ALS

In an eighth embodiment, the multizyme of the invention can be used such that a product of an enzymatic reaction of the multizyme is channeled into a transporter activity which is comprised by the multizyme. The efficiency of transport across a membrane and utilization of the transported molecule may be increased by linking the transporter to an activity that utilizes the transported molecule. For example, nitrogen utilization of a plant may be increased by linker a nitrate transporter to a nitrate reductase activity. A NR/NT multizyme can be created by joining a soluble nitrate reductase (NR) to a nitrate transporter (NT) using a linker derived from a sequence of a DHA synthase locus of a Euglenoid. The fusion of a nitrate transporter to a root nitrate reductase may increase the nitrate uptake from the root environment into the root cells and immediately convert the nitrate to nitrite which is then available for further assimilation of nitrogen compounds such as amides in the root. A similar NR/NT fusion can be produced for above ground processes resulting in an increased nitrate uptake from the xylem cells into the mesohyll cells of leaves coupled with a conversion of nitrate to nitrite which is then available for further assimilation of nitrogen compounds such as amino acids in vegetative tissues. The specification of expression of the multizyme will depend on the promoter used to drive the gene fusion as explained above.

A multizyme of the invention can be used such that at least one activity of the multizyme that is capable of multiple functions when expressed as an individual polypeptide is restricted to a single function when it is part of the multizyme. Many activities in secondary metabolism and fatty acid metabolism are capable of utilizing multiple substrates to produce multiple products. If the substrate presented to a target activity is limited to a single species, the product of a second activity linked in a multizyme, then the number of different products produced by the target activity will be limited to one, desired product. For example, a type II acyl-ACP thioesterase, which is normally active on palmitoyl-ACPI and stearoyl-ACP can be linked to a beta-ketoacyl synthase II such that the thioesterfase is only presented with stearoyl-ACP.

A multizyme of the invention can be used such that at least two enzymatic activities which when linked together have resistance to an herbicide inhibitor of at least one of the individual activities. For example, the multizyme can comprise a 4-hydroxyphenyl pyruvate dioxygenase linked to an activity that produces 4-hydroxyphenylpyruvate or it can comprises a 4-hydroxyphenyl pyruvate dioxygenase linked to a prephenate dehydrogenase or a bifunctional chorismate mutase/PDH.

Expression of a multizyme of the invention in a transgenic plant can confer an increase in seed yield or drought resistance of said plant. For example, increased cytokinin production may be engineered by linking an adenosine phosphate isopentenyltransferase activity to a source of dimethylallyl diphosphate (DMAPP) such as a 1-Hydroxy-2-methyl-2-(E)-butenyl 4-diphosphate reductase thus increasing flux into cytoklnin biosynthesis and protecting DMAPP from other activities such as cis prenyltransferase activity.

Other embodiments include the following:

a multizyme comprising a 1-hydroxy-2-methyl-2-(E)-butenyl4-diphosphate reductase linked to adenosine phosphate-isopentenyltransferase;

a multizyme comprising an herbicide resistant acetolactate synthase peptide linked to a second herbicide resistant acetolactate synthase peptide;

a multizyme comprising a soluble nitrate reductase linked to a nitrate transporter peptide; or

a multizyme comprising an acyl-ACP-thioesterase protein linked to a beta-ketoacyl-ACP synthetase II protein.

### Plant Expression Systems, Cassettes and Vectors, and Transformation

A recombinant DNA construct can be made that contain isolated polynucleotides encoding for the independent and separable activities of the multizyme wherein the independent and separable activities are joined together using a linker derived from a sequence of a DHA synthase locus of a Euglenoid, and wherein the isolated polynucleotides are operably linked to at least one regulatory sequence suitable for expression in a host cell such as a plant.

A promoter is a DNA sequence that directs cellular machinery of a plant to produce RNA from the contiguous coding sequence downstream (3′) of the promoter. The promoter region influences the rate, developmental stage, and cell type in which the RNA transcript of the gene is made. The RNA transcript is processed to produce mRNA which serves as a template for translation of the RNA sequence into the amino acid sequence of the encoded polypeptide. The 5′ non-translated leader sequence is a region of the mRNA upstream of the protein coding region that may play a role in initiation and translation of the mRNA. The 3′ transcription termination/polyadenylation signal is a non-translated region downstream of the protein coding region that functions in the plant cell to cause termination of the RNA transcript and the addition of polyadenylate nucleotides to the 3′ end of the RNA.

The origin of the promoter chosen to drive expression of the multizyme coding sequence is not important as long as it has sufficient transcriptional activity to accomplish the invention by expressing translatable mRNA for the desired nucleic acid fragments in the desired host tissue at the right time. Either heterologous or non-heterologous (i.e., endogenous) promoters can be used to practice the invention. For example, suitable promoters in plants include, but are not limited to:

the alpha prime subunit of beta conglycinin promoter, the Kunitz trypsin inhibitor 3 promoter, the annexin promoter, the glycinin Gy1 promoter, the beta subunit of beta conglycinin promoter, the P34/Gly Bd m 30K promoter, the albumin promoter, the Leg A1 promoter and the Leg A2 promoter.

The annexin, or P34, promoter is described in PCT Publication No. WO 2004/071178 (published Aug. 26, 2004). The level of activity of the annexin promoter is comparable to that of many known strong promoters, such as: (1) the CaMV 35S promoter (Atanassova et al., Plant Mol. Biol. 37:275-285 (1998); Battraw and Hall, Plant Mol. Biol. 15:527-538 (1990); Holtorf et al., Plant Mol. Biol. 29:637-646 (1995); Jefferson et al., EMBO J. 6:3901-3907 (1987); Wilmink et al., Plant Mol. Biol. 28:949-955 (1995)); (2) the Arabidopsis oleosin promoters (Plant et al., Plant Mol. Biol. 25:193-205 (1994); Li, Texas A&M University Ph.D. dissertation, pp. 107-128 (1997)); (3) the Arabidopsis ubiquitin extension protein promoters (Callis et al., J Biol. Chem. 265(21):12486-93 (1990)); (4) a tomato ubiquitin gene promoter (Rollfinke et al., Gene. 211(2):267-76 (1998)); (5) a soybean heat shock protein promoter (Schoffl et al., Mol Gen Genet. 217(2-3):246-53 (1989)); and, (6) a maize H3 histone gene promoter (Atanassova et al., Plant Mol Biol. 37(2):275-85 (1989)).

Another useful feature of the annexin promoter is its expression profile in developing seeds. The annexin promoter is most active in developing seeds at early stages (before 10 days after pollination) and is largely quiescent in later stages. The expression profile of the annexin promoter is different from that of many seed-specific promoters, e.g., seed storage protein promoters, which often provide highest activity in later stages of development (Chen et al., Dev. Genet. 10:112-122 (1989); Ellerstrom et al., Plant Mol. Biol. 32:1019-1027 (1996); Keddie et al., Plant Mol. Biol. 24:327-340 (1994); Plant et al., (supra); Li, (supra)). The annexin promoter has a more conventional expression profile but remains distinct from other known seed specific promoters. Thus, the annexin promoter will be a very attractive candidate when overexpression, or suppression, of a gene in embryos is desired at an early developing stage. For example, it may be desirable to overexpress a gene regulating early embryo development or a gene involved in the metabolism prior to seed maturation.

Following identification of an appropriate promoter suitable for expression of at least two independent and separable activities of the multizyme, the promoter is then operably linked in a sense orientation using conventional means well known to those skilled in the art.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”). For example, a fusion gene can be constructed by linking at least two DNA fragments in frame so as not to introduce a stop codon (in-frame fusion). The resulting fusion gene will be such that each DNA fragment encodes for at least one independent and separable enzymatic activity.

Once the recombinant construct has been made, it may then be introduced into a plant cell of choice by methods well known to those of ordinary skill in the art (e.g., transfection, transformation and electroporation). Expression in a plant cell may be accomplished in a transient or stable fashion as is described above. Methods for transforming dicots (primarily by use of Agrobacterium tumefaciens) and obtaining transgenic plants have been published, among others, for: cotton (U.S. Pat. No. 5,004,863; U.S. Pat. No. 5,159,135); soybean (U.S. Pat. No. 5,569,834; U.S. Pat. No. 5,416,011); Brassica (U.S. Pat. No. 5,463,174); peanut (Cheng et al. Plant Cell Rep. 15:653-657 (1996); McKently et al. Plant Cell Rep. 14:699-703 (1995)); papaya (Ling, K. et al. Bio/technology 9:752-758 (1991)); and pea (Grant et al. Plant Cell Rep. 15:254-258 (1995)). For a review of other commonly used methods of plant transformation see Newell, C. A. (Mol. Biotechnol. 16:53-65 (2000)). One of these methods of transformation uses Agrobacterium rhizogenes (Tepfler, M. and Casse-Delbart, F. Microbiol. Sci. 4:24-28 (1987)). Transformation of soybeans using direct delivery of DNA has been published using PEG fusion (PCT Publication No. WO 92/17598), electroporation (Chowrira, G. M. et al., Mol. Biotechnol. 3:17-23 (1995); Christou, P. et al., Proc. Natl. Acad. Sci. U.S.A. 84:3962-3966 (1987)), microinjection and particle bombardement (McCabe, D. E. et. al., Bio/Technology 6:923 (1988); Christou et al., Plant Physiol. 87:671-674 (1988)). The choice of transformation protocols used for generating transgenic plants and plant cells can vary depending on the type of plant or plant cell, i.e., monocot or dicot, targeted for transformation. Examples of transformation protocols particularly suited for a particular plant type include those for: potato (Tu et al., 1998, Plant Molecular Biology 37:829-838; Chong et al., 2000, Transgenic Research 9:71-78); soybean (Christou et al., 1988, Plant Physiol. 87:671-674; McCabe et al., 1988, BioTechnology 6:923-926; Finer and McMullen, 1991, In Vitro Cell Dev. Biol. 27P:175-182; Singh et al., 1998, Theor. Appl. Genet. 96:319-324); maize (Klein et al., 1988, Proc. Natl. Acad. Sci. 85:4305-4309; Klein et al., 1988, Biotechnology 6:559-563; Klein et al., 1988, Plant Physiol. 91:440-444; Fromm et al., 1990, Biotechnology 8:833-839; Tomes et al., 1995, “Direct DNA Transfer into Intact Plant Cells via Microprojectile Bombardment,” in Plant Cell, Tissue, and Organ Culture: Fundamental Methods, ed. Gamborg (Springer-Verlag, Berlin)); cereals (Hooykaas-Van Slogteren et al., 1984, Nature 311:763-764; U.S. Pat. No. 5,736,369).

The transformed plant cell is then cultured and regenerated under suitable conditions permitting expression multizyme which is then optionally recovered and purified.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, In: Methods for Plant Molecular Biology, (Eds.), Academic: San Diego, Calif. (1988)). This regeneration and growth process typically includes the steps of selection of transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

In addition to the above discussed procedures, practitioners are familiar with the standard resource materials which describe specific conditions and procedures for: the construction, manipulation and isolation of macromolecules (e.g., DNA molecules, plasmids, etc.); the generation of recombinant DNA fragments and recombinant expression constructs; and, the screening and isolating of clones. See, for example: Sambrook et al., Molecular Cloning: A Laboratory Manual, Cold Spring Harbor: N.Y. (1989); Maliga et al., Methods in Plant Molecular Biology, Cold Spring Harbor: N.Y. (1995); Birren et al., Genome Analysis: Detecting Genes, Vol. 1, Cold Spring Harbor: N.Y. (1998); Birren et al., Genome Analysis: Analyzing DNA, Vol. 2, Cold Spring Harbor: N.Y. (1998); Plant Molecular Biology: A Laboratory Manual, eds. Clark, Springer: N.Y. (1997).

Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in plant or in a plant organ, tissue or cell culture.

The term “plant organ” refers to plant tissue or a group of tissues that constitute a morphologically and functionally distinct part of a plant. The term “genome” refers to the following: (1) the entire complement of genetic material (genes and non-coding sequences) that is present in each cell of an organism, or virus or organelle; and/or (2) a complete set of chromosomes inherited as a (haploid) unit from one parent.

Examples of oilseed plants include, but are not limited to: soybean, Brassica species, sunflower, maize, cotton, flax and safflower.

Irrespective of the host selected for expression of a multizyme of the invention, multiple transformants must be screened in order to obtain a strain displaying the desired expression level and pattern. Such screening may be accomplished by Southern analysis of DNA blots (Southern, J. Mol. Biol., 98:503 (1975)), Northern analysis of mRNA expression (Kroczek, J. Chromatogr. Biomed. Appl., 618(1-2):133-145 (1993)), Western and/or Elisa analyses of protein expression, phenotypic analysis, or GC analysis of the PUFA products.

### Examples

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is hereby incorporated by reference in its entirety.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kB” means kilobase(s).

### General Methods:

### Nomenclature for Expression Cassettes:

The structure of an expression cassette will be represented by a simple notation system of “X::Y::Z”, wherein X describes the promoter fragment, Y describes the gene coding region fragment, and Z describes the terminator fragment, which are all operably linked to one another.

The identification of DHA synthase 1 (EgDHAsyn1) and DHA synthase 2 (EgDHAsyn2) from Euglena gracilis cDNA Library eeg1c is described in Applicants' Assignee's Application No. 12/061,738 (filed Apr. 3, 2008, which published Oct. 16, 2008; Attorney Docket No. BB-1585 USNA), the subject matter of which is hereby incorporated by reference in its entirety.

### Example 1

### Primary Structure Analysis of EqC20elo1, EqDHAsyn1 and EqDHAsyn2

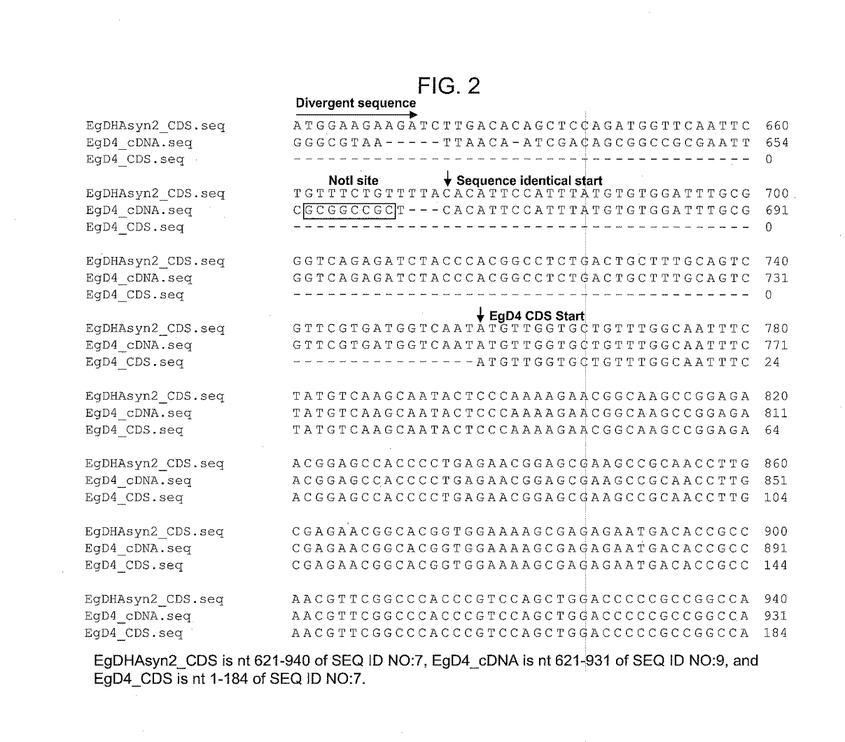

Given the 100% amino acid identity between the C-terminus of EgDHAsyn2 (SEQ ID NO:8) and the Euglena gracilis delta-4 desaturase (SEQ ID NO:6), a nucleotide sequence alignment was carried out between the coding sequence of EgDHAsyn2 (SEQ ID NO:7), the cDNA sequence of the Euglena gracilis delta-4 desaturase (SEQ ID NO:9) (NCBI Accession No. AY278558 (GI 33466345), locus AY278558, Meyer et al., Biochemistry 42(32):9779-9788 (2003)) and the coding sequence of the Euglena gracilis delta-4 desaturase (SEQ ID NO:10) (Meyer et al., supra). Sequence alignment was performed by the Clustal W method (using the MegAlign™ v6.1 program of the LASERGENE bioinformatics computing suite (DNASTAR Inc.) with the default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). The alignment is shown in FIG. 2. The Euglena gracilis delta-4 desaturase coding sequence is named EgD4_CDS (SEQ ID NO:10), the Euglena gracilis delta-4 desaturase cDNA sequence is named EgD4_cDNA (SEQ ID NO:9) and the Euglena gracilis DHA synthase 2 coding sequence is named EgDHAsyn2_CDS (SEQ ID NO:7).

The 5′ end (where the sequences are divergent) and the 3′ end (where the sequences are identical) of the alignment are truncated in order to fit the alignment on one page. FIG. 2 illustrates that the sequences are highly divergent from the start of the Euglena gracilis delta-4 desaturase cDNA to 83 by upstream of the coding sequence (CDS) start site. It is clear from the alignment that the nucleotide sequences for EgD4_cDNA and EgDHAsyn2_CDS are identical from 83 by upstream of the CDS start site of the Euglena gracilis delta-4 desaturase cDNA sequence (SEQ ID NO:9), which is equivalent to nucleotide 674 of the EgDHAsyn2_CDS (SEQ ID NO:7), through to the end of the sequences. At the exact point of divergence, a NotI site can be found in the Euglena gracilis cDNA sequence (nucleotides 656-663 of SEQ ID NO:9) and since NotI linkers were used in the original cloning of the Euglena gracilis delta-4 desaturase cDNA (see Meyer et al., supra) it is likely that what was cloned was an incomplete, not full-length, transcript for EgDHAsyn2.

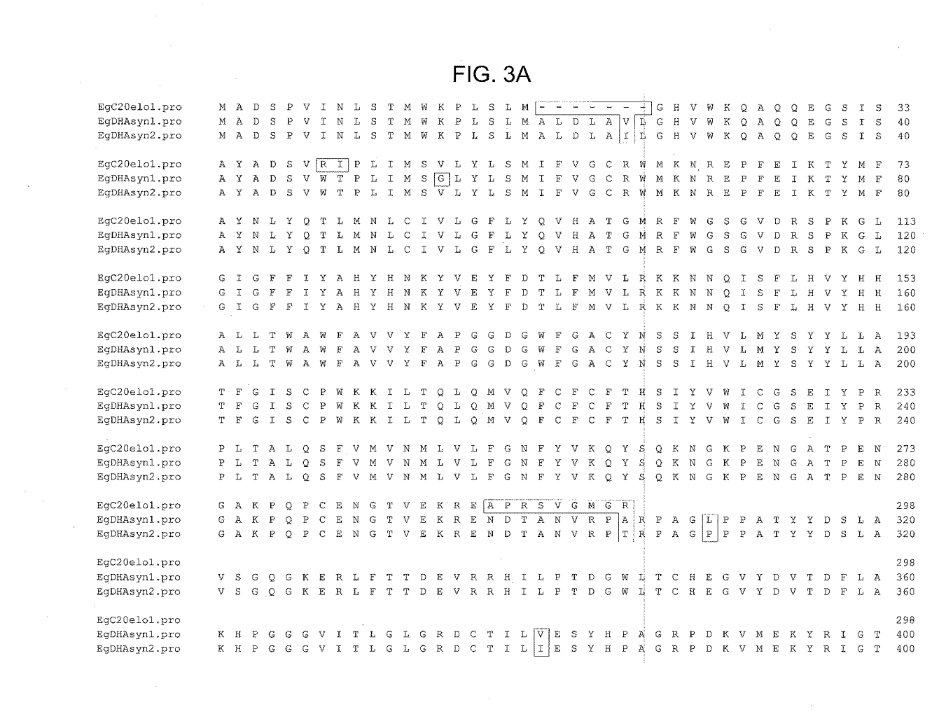

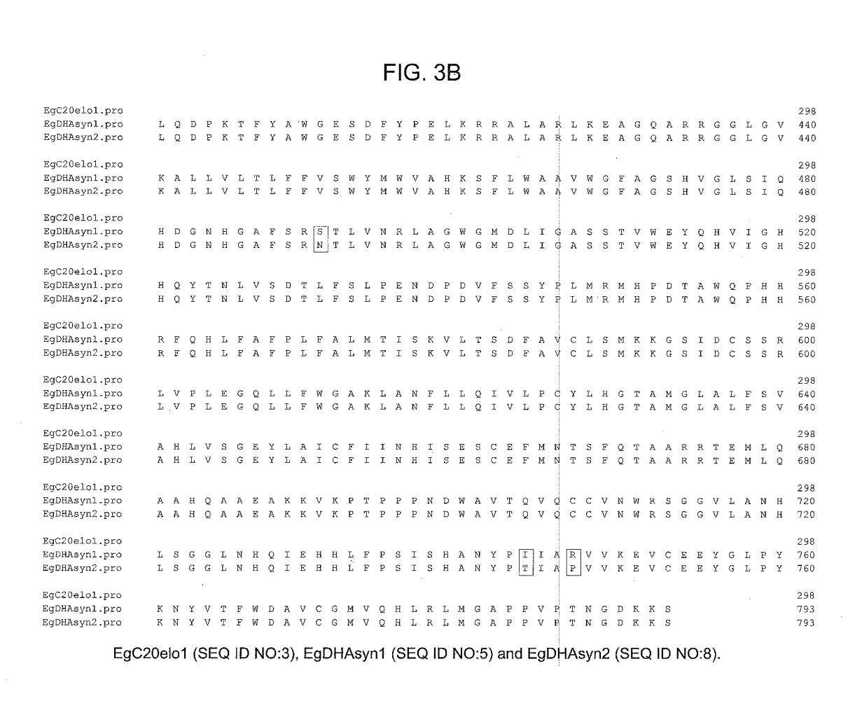

The amino acid sequence EgDHAsyn1 (SEQ ID NO:5) was compared to EgDHAsyn2 (SEQ ID NO:8) and EgC20elo1 (SEQ ID NO:3) using the Clustal W method as described above and the alignment is shown in FIGS. 3A and 3B. Compared to EgDHAsyn1 and EgDHAsyn2, EgC20elo1 has a deletion of 7 amino acids (i.e., A L D L A [V/I] L) and 2 other amino acid substitutions (i.e., W47R, T48I; based on numbering for EgDHAsyn1) at the N-terminus. After amino acid 289 of EgC20elo1, the sequences are very different when compared to the DHA synthases with EgDHAsyn1 and EgDHAsyn2 having an additional 498 amino acids at the C-terminus with homology to delta-4 fatty acid desaturases while EgC20elo1 ends after only 9 additional amino acids. The amino acid sequences of EgDHAsyn1 (SEQ ID NO:5) and EgDHAsyn2 (SEQ ID NO:8) have 8 amino acid differences between the 2 sequences (i.e., V25I, G54V, A305T, L310P, V380I, S491N, I744T, R747P; based on numbering for EgDHAsyn1). The last four differences occur in the delta-4 desaturase domain.

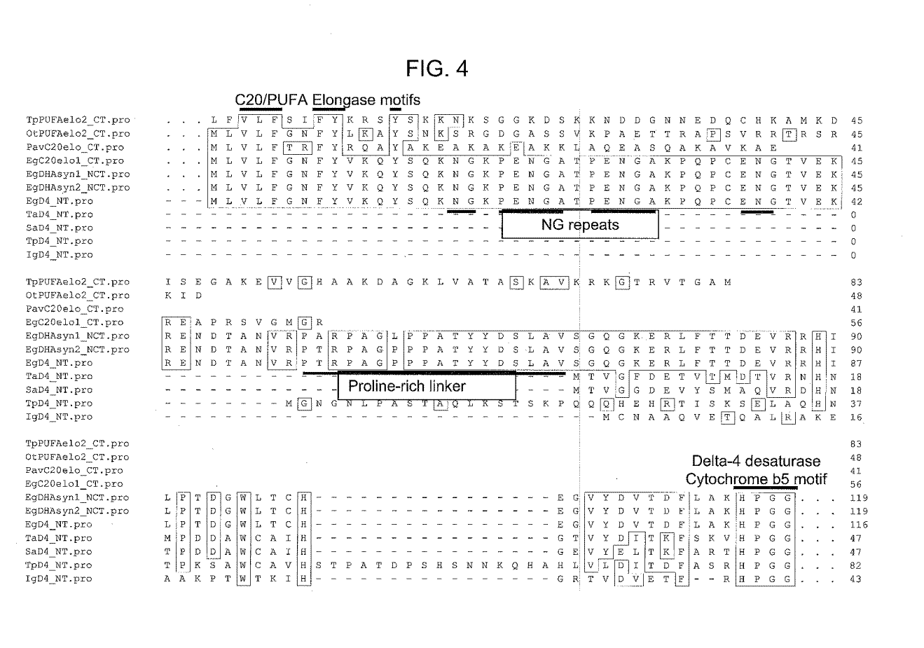

FIG. 4 shows an alignment of an interior fragment of EgDHAsyn1 (labeled as “EgDHAsyn1_ NCT.pro”; amino acids 253-365 of SEQ ID NO:5) and EgDHAsyn2 (labeled as “EgDHAsyn2_NCT.pro”; amino acids 253-365 of SEQ ID NO:8) spanning both the C20 elongase region and the delta-4 desaturase domain (based on homology) with the C-termini of C20 elongases (EgC20elo1_CT.pro, amino acids 246-298 of SEQ ID NO:3; PavC20elo_CT.pro, amino acids 240-277 of SEQ ID NO:2; OtPUFAelo2_CT.pro, amino acids 256-300 of SEQ ID NO:11; TpPUFAelo2_CT.pro, amino acids 279-358 of SEQ ID NO:12) and the N-termini of delta-4 desaturases (EgD4_NT.pro, amino acids 1-116 of SEQ ID NO:6; TaD4_NT.pro, amino acids 1-47 of SEQ ID NO:13; SaD4_NT.pro, amino acids 1-47 of SEQ ID NO:14; TpD4_NT.pro, amino acids 1-82 of SEQ ID NO:15; IgD4_NT.pro, amino acids 1-43 of SEQ ID NO:16) is shown. A conserved motif at the C-terminus of all the C20 elongase domains (i.e., VLFXXFYXXXY (SEQ ID NO:29)) is also present at the N-terminus of EgD4 and further supports EgD4 being an incomplete DHA synthase.

At the C-terminus of the C20 elongase domain of EgDHAsyn1, EgDHAsyn2 and EgC20elo1, there is a repeated sequence containing an NG motif (i.e., KNGK (SEQ ID NO:30), PENGA (SEQ ID NO:31), PCENGTV (SEQ ID NO:32); called NG repeats and indicated in FIG. 4 with lines under the sequence). Although the pattern occurs with a high probability of occurrence, a scan of the NG repeated region using Prosite shows the last NG motif (i.e., NGTV) in this region as a potential N-glycosylation site. After the NG repeat region, both EgDHAsyn1 and EgDHAsyn2 contain a proline-rich region (labeled “Proline-rich linker” in FIG. 4), which may act as a linker between the C20 elongase and delta-4 desaturase domains. The linker may play a role in keeping the C20 elongase and delta-4 desaturase domains in the proper structural orientation to allow efficient conversion of EPA to DHA. Although the proline-rich linker is shown in FIG. 4 as extending from P304 to V321 (based on numbering for EgDHAsyn1), the NG repeat region is also somewhat proline-rich and may also play a role in this linker function.

The nucleotide and corresponding amino acid sequences for the proline-rich linker of EgDHAsyn1, as defined in FIG. 4, are set forth in SEQ ID NO:33 and SEQ ID NO:34, respectively. The nucleotide and corresponding amino acid sequences for the proline-rich linker of EgDHAsyn2, as defined in FIG. 4, are set forth in SEQ ID NO:35 and SEQ ID NO:36, respectively.

The nucleotide and corresponding amino acid sequences for the EgDHAsyn1 C20 elongase domain from EgDHAsyn1 are set forth in SEQ ID NO:201 and SEQ ID NO:202, respectively. The nucleotide and corresponding amino acid sequences for the EgDHAsyn2 C20 elongase domain are set forth in SEQ ID NO:203 and SEQ ID NO:204, respectively.

### Example 2

### Production and Model System Transformation of Somatic Soybean Embryo Cultures with Soybean Expression Vectors

### Culture Conditions:

Soybean embryogenic suspension cultures (cv. Jack) are maintained in 35 mL liquid medium SB196 (infra) on a rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 mL of fresh liquid SB196 (the preferred subculture interval is every 7 days).

Soybean embryogenic suspension cultures are transformed with the soybean expression plasmids by the method of particle gun bombardment (Klein et al., Nature 327:70 (1987)) using a DuPont Biolistic PDS1000/HE instrument (helium retrofit) for all transformations.

### Soybean Embryogenic Suspension Culture Initiation:

Soybean cultures are initiated twice each month with 5-7 days between each initiation. Pods with immature seeds from available soybean plants are picked 45-55 days after planting. Seeds are removed from the pods and placed into a sterilized magenta box. The soybean seeds are sterilized by shaking them for 15 min in a 5% Clorox solution with 1 drop of Ivory soap (i.e., 95 mL of autoclaved distilled water plus 5 mL Clorox and 1 drop of soap, mixed well). Seeds are rinsed using 2 1-liter bottles of sterile distilled water and those less than 4 mm are placed on individual microscope slides. The small end of the seed is cut and the cotyledons are pressed out of the seed coat. When cultures are being prepared for production transformation, cotyledons are transferred to plates containing SB1 medium (25-30 cotyledons per plate). Plates are wrapped with fiber tape and are maintained at 26° C. with cool white fluorescent lights on 16:8 h day/night photoperiod at light intensity of 60-80 μE/m2/s for eight weeks, with a media change after 4 weeks. When cultures are being prepared for model system experiments, cotyledons are transferred to plates containing SB199 medium (25-30 cotyledons per plate) for 2 weeks, and then transferred to SB1 for 2-4 weeks. Light and temperature conditions are the same as described above. After incubation on SB1 medium, secondary embryos are cut and placed into SB196 liquid media for 7 days.

### Preparation of DNA for Bombardment:

Either an intact plasmid or a DNA plasmid fragment containing the genes of interest and the selectable marker gene are used for bombardment. Fragments from soybean expression plasmids, the construction of which is described herein, are obtained by gel isolation of digested plasmids. In each case, 100 μg of plasmid DNA is used in 0.5 mL of the specific enzyme mix described below. Plasmids are digested with AscI (100 units) in NEBuffer 4 (20 mM Tris-acetate, 10 mM magnesium acetate, 50 mM potassium acetate, 1 mM dithiothreitol, pH 7.9), 100 μg/mL BSA, and 5 mM beta-mercaptoethanol at 37° C. for 1.5 hr. The resulting DNA fragments are separated by gel electrophoresis on 1% SeaPlaque GTG agarose (BioWhitaker Molecular Applications) and the DNA fragments containing gene cassettes are cut from the agarose gel. DNA is purified from the agarose using the GELase digesting enzyme following the manufacturer's protocol.

A 50 μL aliquot of sterile distilled water containing 3 mg of gold particles (3 mg gold) is added to 30 μL of a 10 ng/μL DNA solution (either intact plasmid or DNA fragment prepared as described herein), 25 μL 5M CaCl₂ and 20 μL of 0.1 M spermidine. The mixture is shaken 3 min on level 3 of a vortex shaker and spun for 10 sec in a bench microfuge. The supernatant is removed, followed by a wash with 400 μL 100% ethanol and another brief centrifugation. The 400 ul ethanol is removed and the pellet is resuspended in 40 μL of 100% ethanol. Five μL of DNA suspension is dispensed to each flying disk of the Biolistic PDS1000/HE instrument disk. Each 5 μL aliquot contained approximately 0.375 mg gold per bombardment (e.g., per disk).

For model system transformations, the protocol is identical except for a few minor changes (i.e., 1 mg of gold particles is added to 5 μL of a 1 μg/μL DNA solution, 50 μL of a 2.5M CaCl₂ is used and the pellet is ultimately resuspended in 85 μL of 100% ethanol thus providing 0.058 mg of gold particles per bombardment).

Tissue Preparation and Bombardment with DNA:

Approximately 150-200 mg of seven day old embryogenic suspension cultures is placed in an empty, sterile 60×15 mm petri dish and the dish is covered with plastic mesh. The chamber is evacuated to a vacuum of 27-28 inches of mercury, and tissue is bombarded one or two shots per plate with membrane rupture pressure set at 1100 PSI. Tissue is placed approximately 3.5 inches from the retaining/stopping screen. Model system transformation conditions are identical except 100-150 mg of embryogenic tissue is used, rupture pressure is set at 650 PSI and tissue is place approximately 2.5 inches from the retaining screen.

### Selection of Transformed Embryos:

Transformed embryos are selected either using hygromycin (when the hygromycin B phosphotransferase (HPT) gene is used as the selectable marker) or chlorsulfuron (when the acetolactate synthase (ALS) gene is used as the selectable marker).

Following bombardment, the tissue is placed into fresh SB196 media and cultured as described above. Six to eight days post-bombardment, the SB196 is exchanged with fresh SB196 containing either 30 mg/L hygromycin or 100 ng/mL chlorsulfuron, depending on the selectable marker used. The selection media is refreshed weekly. Four to six weeks post-selection, green, transformed tissue is observed growing from untransformed, necrotic embryogenic clusters.

### Embryo Maturation:

For production transformations, isolated, green tissue is removed and inoculated into multiwell plates to generate new, clonally propagated, transformed embryogenic suspension cultures. Transformed embryogenic clusters are cultured for four-six weeks in multiwell plates at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 μE/m²s. After this time embryo clusters are removed to a solid agar media, SB166, for one-two weeks and then subcultured to SB103 medium for 3-4 weeks to mature embryos. After maturation on plates in SB103, individual embryos are removed from the clusters, dried and screened for alterations in their fatty acid compositions as described supra.

For model system transformations, embryos are matured in soybean histodifferentiation and maturation liquid medium (SHaM liquid media; Schmidt et al., Cell Biology and Morphogenesis 24:393 (2005)) using a modified procedure. Briefly, after 4 weeks of selection in SB196 as described above, embryo clusters are removed to 35 mL of SB228 (SHaM liquid media) in a 250 mL Erlenmeyer flask. Tissue is maintained in SHaM liquid media on a rotary shaker at 130 rpm and 26° C. with cool white fluorescent lights on a 16:8 hr day/night photoperiod at a light intensity of 60-85 μE/m2/s for 2 weeks as embryos matured. Embryos grown for 2 weeks in SHaM liquid media are equivalent in size and fatty acid content to embryos cultured on SB166/SB103 for 5-8 weeks.

After maturation in SHaM liquid media, individual embryos are removed from the clusters, dried and screened for alterations in their fatty acid compositions as described supra.

### Media Recipes:

SB 196 - FN Lite Liquid Proliferation Medium (per liter)

| | | |
|---|---|---|
| MS FeEDTA - 100x Stock 1 | 10 | mL |
| MS Sulfate - 100x Stock 2 | 10 | mL |
| FN Lite Halides - 100x Stock 3 | 10 | mL |
| FN Lite P, B, Mo - 100x Stock 4 | 10 | mL |
| B5 vitamins (1 mL/L) | 1.0 | mL |
| 2,4-D (10 mg/L final concentration) | 1.0 | mL |
| KNO₃ | 2.83 | gm |
| (NH₄)₂SO₄ | 0.463 | gm |
| asparagine | 1.0 | gm |
| sucrose (1%) | 10 | gm |
| pH 5.8 | | |

FN Lite Stock Solutions

| Stock | | | | | |
|---|---|---|---|---|---|
| Number | | 1000 mL | | 500 mL | |
| 1 | MS Fe EDTA 100x Stock | | | | |
| | Na₂ EDTA* | 3.724 | g | 1.862 | g |
| | FeSO₄—7H₂O | 2.784 | g | 1.392 | g |
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100x Stock | | | | |
| | CaCl₂—2H₂O | 30.0 | g | 15.0 | g |
| | KI | 0.083 | g | 0.0715 | g |
| | CoCl₂—6H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

*Add first, dissolve in dark bottle while stirring

### SB1 Solid Medium (Per Liter)

1 package MS salts (Gibco/BRL—Cat. No. 11117-066)

1 mL B5 vitamins 1000× stock

31.5 g glucose

2 mL 2,4-D (20 mg/L final concentration)

pH 5.7

8 g TC agar

### SB199 Solid Medium (Per Liter)

1 package MS salts (Gibco/BRL—Cat. No. 11117-066)

1 mL B5 vitamins 1000× stock

30g Sucrose

4 ml 2,4-D (40 mg/L final concentration)

pH 7.0

2 gm Gelrite

### SB 166 Solid Medium (Per Liter)

1 package MS salts (Gibco/BRL—Cat. No. 11117-066)

1 mL B5 vitamins 1000× stock

60 g maltose

750 mg MgCl₂ hexahydrate

5 g activated charcoal

pH 5.7

2 g gelrite

### SB 103 Solid Medium (Per Liter)

1 package MS salts (Gibco/BRL—Cat. No. 11117-066)

1 mL B5 vitamins 1000× stock

60 g maltose

750 mg MgCl₂ hexahydrate

pH 5.7

2 g gelrite

### SB 71-4 Solid Medium (Per Liter)

1 bottle Gamborg's B5 salts w/sucrose (Gibco/BRL—Cat. No. 21153-036)

pH 5.7

5 g TC agar

### 2,4-D Stock

Obtain premade from Phytotech Cat. No. D 295—concentration 1 mg/mL

### B5 Vitamins Stock (Per 100 mL)

Store aliquots at −20° C.
10 g myo-inositol
100 mg nicotinic acid
100 mg pyridoxine HCI
1 g thiamine

If the solution does not dissolve quickly enough, apply a low level of heat via the hot stir plate.

SB 228- Soybean Histodifferentiation & Maturation (SHaM) (per liter)

| | | |
|---|---|---|
| DDI H₂O | 600 | mL |
| FN-Lite Macro Salts for SHaM 10X | 100 | mL |
| MS Micro Salts 1000x | 1 | mL |
| MS FeEDTA 100x | 10 | mL |
| CaCl 100x | 6.82 | mL |
| B5 Vitamins 1000x | 1 | mL |
| L-Methionine | 0.149 | g |
| Sucrose | 30 | g |
| Sorbitol | 30 | g |

Adjust volume to 900 mL

pH 5.8

Autoclave

Add to cooled media (≦30° C.):

| | |
|---|---|
| *Glutamine (final concentration 30 mM) 4% | 110 mL |

*Note: Final volume will be 1010 mL after glutamine addition.
Since glutamine degrades relatively rapidly, it may be preferable to add immediately prior to using media. Expiration 2 weeks after glutamine is added; base media can be kept longer without glutamine.

FN-lite Macro for SHAM 10X- Stock #1 (per liter)

| | |
|---|---|
| (NH₄)₂SO₄ (ammonium sulfate) | 4.63 g |
| KNO₃ (potassium nitrate) | 28.3 g |
| MgSO₄*7H₂0 (magnesium sulfate heptahydrate) | 3.7 g |
| KH₂PO₄ (potassium phosphate, monobasic) | 1.85 g |

Bring to volume

Autoclave

MS Micro 1000X- Stock #2 (per 1 liter)

| | | |
|---|---|---|
| H₃BO₃ (boric acid) | 6.2 | g |
| MnSO₄*H₂O (manganese sulfate monohydrate) | 16.9 | g |
| ZnSO4*7H₂0 (zinc sulfate heptahydrate) | 8.6 | g |
| Na₂MoO₄*2H₂0 (sodium molybdate dihydrate) | 0.25 | g |
| CuSO₄*5H₂0 (copper sulfate pentahydrate) | 0.025 | g |
| CoCl₂*6H₂0 (cobalt chloride hexahydrate) | 0.025 | g |
| KI (potassium iodide) | 0.8300 | g |

Bring to volume

Autoclave

FeEDTA 100X- Stock #3 (per liter)

| | |
|---|---|
| Na₂EDTA* (sodium EDTA) | 3.73 g |
| FeSO₄*7H₂0 (iron sulfate heptahydrate) | 2.78 g |

*EDTA must be completely dissolved before adding iron.

Bring to Volume

Solution is photosensitive. Bottle(s) should be wrapped in foil to omit light.

Autoclave

Ca 100X- Stock #4 (per liter)

| | |
|---|---|
| CaCl₂*2H₂0 (calcium chloride dihydrate) | 44 g |

Bring to Volume

Autoclave

B5 Vitamin 1000X- Stock #5 (per liter)

| | |
|---|---|
| Thiamine*HCl | 10 g |
| Nicotinic Acid | 1 g |
| Pyridoxine*HCl | 1 g |
| Myo-Inositol | 100 g |

Bring to Volume

Store frozen

4% Glutamine- Stock #6 (per liter)

| | |
|---|---|
| DDI water heated to 30° C. | 900 mL |
| L-Glutamine | 40 g |

Gradually add while stirring and applying low heat.

Do not exceed 35° C.

Bring to Volume

Filter Sterilize

Store frozen*

*Note: Warm thawed stock in 31° C. bath to fully dissolve crystals.

### Example 3

### Chlorsulfuron Selection (ALS) and Plant Regeneration

### Chlorsulfuron (ALS) Selection:

Following bombardment, the tissue is divided between 2 flasks with fresh SB196 media and cultured as described in Example 2. Six to seven days post-bombardment, the SB196 is exchanged with fresh SB196 containing selection agent of 100 ng/mL chlorsulfuron (chlorsulfuron stock is 1 mg/mL in 0.01 N ammonium hydroxide). The selection media is refreshed weekly. Four to six weeks post selection, green, transformed tissue may be observed growing from untransformed, necrotic embryogenic clusters. Isolated, green tissue is removed and inoculated into multiwell plates containing SB196 and embryos are matured as described in Example 2.

Regeneration of Soybean Somatic Embryos into Plants:

In order to obtain whole plants from embryogenic suspension cultures, the tissue must be regenerated. Embryos are matured as described in Example 2. After subculturing on medium SB103 for 3 weeks, individual embryos can be removed from the clusters and screened for alterations in their fatty acid compositions as described herein. It should be noted that any detectable phenotype, resulting from the expression of the genes of interest, could be screened at this stage. This would include, but not be limited to, alterations in fatty acid profile, protein profile and content, carbohydrate content, growth rate, viability, or the ability to develop normally into a soybean plant.

Matured individual embryos are desiccated by placing them into an empty, small petri dish (35×10 mm) for approximately 4 to 7 days. The plates are sealed with fiber tape (creating a small humidity chamber). Desiccated embryos are planted into SB71-4 medium where they are left to germinate under the same culture conditions described above. Germinated plantlets are removed from germination medium and rinsed thoroughly with water and then are planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for trait of interest.

### Example 4

### Construction of Soybean Expression Vector pKR1183 for Expression of a Euglena anabaena Delta-9 Elongase-Tetruetreptia pomquetensis CCMP1491 Delta-8 Desaturase Fusion Gene (Hybrid1-HGLA Synthase)

An in-frame fusion between the Euglena anabaena delta-9 elongase (EaD9Elo1; SEQ ID NO:11), the Euglena gracilis DHA synthase 1 proline-rich linker (EgDHAsyn1Link; SEQ ID NO:33) and the Tetruetreptia pomquetensis CCMP1491 delta-8 desaturase (TpomD8; SEQ ID NO:26;) was constructed using the conditions described below (see also Applicants' Assignee's application Ser. No. 12/061,738 (filed Apr. 3, 2008, which published Oct. 16, 2008; Attorney Docket No. BB-1585 USNA).

An initial in-frame fusion between the EaD9Elo1 and the EgDHAsyn1Link (EaD9elo-EgDHAsyn1Link) was made, flanked by an NcoI site at the 5′end and a NotI site at the 3′ end, by PCR amplification. EaD9Elo1 (SEQ ID NO:39) was amplified from pLF121-1 (SEQ ID NO:38) with oligonucleotides EaD9-5Bbs (SEQ ID NO:41) and EaD9-3fusion (SEQ ID NO:42), using the Phusion™ High-Fidelity DNA Polymerase (Cat. No. F553S, Finnzymes Oy, Finland) following the manufacturer's protocol. EgDHAsyn1Link (SEQ ID NO:33) was amplified in a similar way from pKR1049 (described in Applicants' Assignee's application having U.S. application Ser. No. 12/061,738 (filed Apr. 3, 2008, which published Oct. 16, 2008; Attorney Docket No. BB-1585 USNA)), with oligonucleotides EgDHAsyn1Link-5fusion (SEQ ID NO:43) and MWG511 (SEQ ID NO:28). The two resulting PCR products were combined and re-amplified using EaD9-5Bbs (SEQ ID NO:41) and MWG511 (SEQ ID NO:28) to form EaD9Elo1-EgDHAsyn1Link. The sequence of the EaD9Elo1-EgDHAsyn1Link is shown in SEQ ID NO:44. EaD9Elo1-EgDHAsyn1Link does not contain an in-frame stop codon upstream of the NotI site at the 3′ end and therefore, a DNA fragment cloned into the NotI site can give rise to an in-frame fusion with the EgD9elo1-EgDHAsyn1Link if the correct frame is chosen. EaD9Elo1-EgDHAsyn1Link was cloned into the pCR-Blunt® cloning vector using the Zero Blunt® PCR Cloning Kit (Invitrogen Corporation), following the manufacturer's protocol, to produce pLF124 (SEQ ID NO:45).

The BbsI/NotI DNA fragment of pLF124 (SEQ ID NO:45), containing EaD9Elo1-EgDHAsyn1Link, was cloned into the NcoI/NotI DNA fragment from KS366 containing the promoter for the α′ subunit of β-conglycinin, to produce pKR1177 (SEQ ID NO:46).

The BamHI DNA fragment of pKR1177 (SEQ ID NO:46), containing EaD9Elo1-EgDHAsyn1Link, was cloned into the BamHI DNA fragment of pKR325, previously described in PCT Publication No. WO 2006/012325 (the contents of which are hereby incorporated by reference) to produce pKR1179 (SEQ ID NO:47).

The NotI fragment from pLF114-10 (SEQ ID NO:27), containing TpomD8 was cloned into the NotI fragment of pKR1179 (SEQ ID NO:47) to produce pKR1183 (SEQ ID NO:48).

### Example 5

### Expression of Chimeric Genes in Monocot Cells

A chimeric gene comprising a fusion gene encoding the instant multizyme in sense orientation with respect to the maize 27 kD zein promoter that is located 5′ to the fusion gene, and the 10 kD zein 3′ end that is located 3′ to the fusion gene, can be constructed. The fusion gene may be generated by polymerase chain reaction (PCR) of cDNA clones using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue; Stratagene). Bacterial transformants can be screened by restriction enzyme digestion of plasmid DNA and limited nucleotide sequence analysis using the dideoxy chain termination method (Sequenase DNA Sequencing Kit; U.S. Biochemical). The resulting plasmid construct would comprise a chimeric gene encoding, in the 5′ to 3′ direction, the maize 27 kD zein promoter, a fusion gene encoding the instant polypeptides, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 6

### Synthesis of Multizymes for Herbicide Resistance

This example describes the synthesis of multizymes wherein the use of the multizyme in a reaction protects an activity of the multizyme from competitive inhibition by a substrate other than that produced by a preceding enzymatic activity of the multizyme.

HPPD (4-hydroxyphenylpyruvate dioxygenase) is a known herbicide target and inhibition of HPPD activity leads to plant bleaching and death (Mayonado et al. (1989) Pestic. Biochem. Physiol. 35:138-145; Schultz et al. (1993) FEBS lett. 318:162-166; Secor (1994) Plant Phys. 106:1429-1433). HPPD inhibitors such as diketonitrile are competitive with p-hydroxyphenylpyruvate, the substrate of HPPD. The level of tolerance to different HPPD-inhibitors may vary depending on the HPPD enzyme. HPPD catalyzes the second step in the pathway for the catabolism of tyrosine, that is common to essentially all aerobic forms of life (Moran G R, Archives of biochemistry and biophysics 2005 433:117-28). In plants, HPPD is essential in the biosynthesis of tocopherols (vitamin E) and plastoquinones, which are involved in electron transport during photosynthesis.

In order to produce plants containing HPPD herbicide resistance, it is desirable to reduce the susceptibility of HPPD to herbicides. Fusing HPPD with a prokaryotic prephenate dehydrogenase (PDH) or bifunctional chorismate mutase/prephenate dehydrogenase or a plant prephenate dehydrogenase (PDH) (or another suitable source of hydroxyphenylpyruvate such as tyrosine aminotransferase) can protect the HPPD from competitive-substrate inhibition and create a herbicide-resistant HPPD activity.

An HPPD/PDH multizyme is created by joining HPPD with a prokaryotic or plant PDH or with a prokaryotic bifunctional chorismate mutase/PDH. An HPPD/tyrosine aminotransferase multizyme is created by joining an HPPD and a plant or microbial tyrosine aminotransferase.

HPPD genes from many organism have been identified to date and are well known in the art. Some examples include, but are not limited to HPPDs from carrots (NCBI GI:2231615), barley (NCBI GI::2695710), and Arabidopsis thaliana (Garcia, I., Rodgers et al. 1999 Plant Physiol. 119:1507-1516) with at least two different variants existing in this last plant; HPPD cDNA's from rice, soybean, Vernonia, Catalpa described in U.S. Pat. No. 7,226,745 (SEQ ID NOs: 11 & 13, 15, 17, 19, 31 of U.S. Pat. No. 7,226,745 respectively) the content of which is incorporated by reference); Mycosphaerella graminicola (Keon J and Hargreaves J. FEMS Microbiol Lett. 1998 Apr. 15; 161(2):337-43.), Human (Awata H, Endo F, Matsuda I. Genomics. 1994 October; 23(3):534-9 and Coccidiodes immitis. (Wyckoff E E, Pishko E J, Kirkland T N, Cole G T. Gene. 1995 Aug. 8; 161(1):107-11.) carrots (NCBI GI:2231615), barley (NCBI GI::2695710), and Arabidopsis thaliana (Garcia, I., Rodgers et al. 1999 Plant Physiol. 119:1507-1516) maize (US 20030066102); cotton, tomato, canola (US 20050289664); wheat, oat, sorghum and other grasses (US 20040058427); Pseudomonas (U.S. Pat. No. 6,245,986). Microbial Prephenate dehydrogenase genes include, but are not limited to, Candida albicans (NCBI GI:3635639), Saccharomyces cerevisiae (NCBI GI: 852464), Frankia sp. EAN1pec(NCBI GI: 5673396), Bacillus licheniformis ATCC 14580 (NCBI GI: 3099695), Bacillus cereus (NCBI GI: 1205287) and Aquifex aeolicus (Bonvin J, et al. Protein Sci. 2006 15(6):1417-32). Plant genes that have been annotated as coding for prephenate dehydrogenase include those in Arabidopsis thaliana (NCBI GI:15218283) and white spruce (NCBI, GI:1350504), but the corresponding enzyme activities have not been demonstrated. Prephenate dehydrogenase activity has not been observed in non-legumes (Siehl, D L, Biosynthesis of Tryptophan, Tyrosine and Phenylalanine from Chorismate, in Plant Amino Acids: Biochemistry and Biotechnology, Marcel Dekker, 1999). Instead, non-legume genes annotated as coding for prephenate dehydrogenase likely are arogenate dehydrogenases. Extracts of soybean leaves have prephenate dehydrogenase activity that can be chromatographically resolved from arogenate dehydrogenase (Siehl, D L, ibid) The soybean genes coding for these activities have not been identified, A tyrosine aminotransferase gene in Arabidopsis thaliana is present in NCBI (GI: 817022).

The independent and separate activities of the HPPD/PDH multizyme or the HPPD/tyrosine aminotransferase multizyme (e.g, HPPD, PDH, bifunctional chorismate mutase/PDH or tyrosine aminotransferase) are joined together using a linker derived from a sequence of a DHA synthase locus of a Euglenoid. An example of such a linker is the EgDHAsyn1 proline-rich linker (SEQ ID NO:34). The gene fusion encoding the multizyme may be produced as described in Example 4, or as described by Examples 15-24 of Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety. Furthermore, any number of methods known to those skilled in the art may be used to produce the gene fusion. Additional nucleotides can be added to the 3′ end of the EgDHAsyn1 proline-rich linker sequence to enable cloning when making the fusions for all constructs. Thus, an additional 4 amino acids can be included between the end of the EgDHAsyn1 proline-rich linker (SEQ ID NO:34; PARPAGLPPATYYDSLAV) and the start of the bifunctional chorismate mutase/PDH, PDH or tyrosine aminotransferase used.

For comparison, constructs that co-expressed the independent and separate activities of the multizyme (HPPD, bifunctional chorismate mutase/PDH, PDH and tryisone aminotransferase) are also created. The multizyme may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the multiple enzymes.

The multizyme fusion gene can be expressed in either microbial cells as described in General Methods, monocot cells as described in EXAMPLE 5 or dicot cells as described in Example 2 and 3, or expression in eukaryotic cell culture, in planta, and using viral expression systems in suitably infected organisms or cell lines. Purification of the instant multizymes, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. The purification protocol may include the use of an affinity resin containing ligands which are specific for the independent and separate activities of the multizyme.

Crude, partially purified or purified enzyme may be utilized in assays for the evaluation of compounds for their ability to inhibit enzymatic activation of the instant multizymes disclosed herein. Assays may be conducted under well known experimental conditions which permit optimal enzymatic activity. For example, assays for HPPD are presented by Norris et al. (1995) Plant Cell 7: 2139-2149; assays for chorismate mutase by Kim S K, et al. 2006 (J Bacteriol. 2006, 188(24):8638-48) and for prephenate dehydrogenase assays by Xu S, et al. 2006. Protein Expr Purif. 49(2):151-8.

### Example 7

### Synthesis of Multizymes for Increased Cytokinin Production

This example describes the synthesis of multizymes having at least two independent and separable activities wherein the independent and separable activities are joined together using a linker derived from a sequence of a DHA synthase locus of a Euglenoid and wherein the product of a first reaction of the multizyme is substantially used in a subsequent reaction of the multizyme.

Cytokinins (CKs) are plant hormones that play a role in cell division, cell differentiaition, release of lateral buds from apical dominance, and delay of senescence. (Mok, D. W., and Mok, M. C. (2001) Annu. Rev. Plant Physiol. Plant Mol. Biol. 52, 89-1181). Most natural CKs, including isopentenyladenine (iP) and trans-zeatin (tZ) are derivatives of N⁶-prenylated adenine. (Mok, D. W., and Mok, M. C. (2001) Annu. Rev. Plant Physiol. Plant Mol. Biol. 52, 89-1181). In CK biosynthesis, the prenylation of AMP, ADP or ATP is catalyzed by adenosine phosphate isopentenyltransferase (adenosine phosphate-IPT; EC 2.5.1.27), which utilizes dimethylallyl diphosphate (DMAPP) as a substrate. This enzyme activity leads to the formation of isopentenyladenine riboside monophosphate (iPRMP) (Taya, Y., Tanaka, Y., and Nishimura, S. (1978) Nature 271, 545-547). Subsequent conversion of iPRMP to iP is catalyzed by 5′-nucleotidase and adenosine nucleosidase (Chen, C.-M., and Kristopeit, S. M. (1981) Plant Physiol. 67, 494-498).

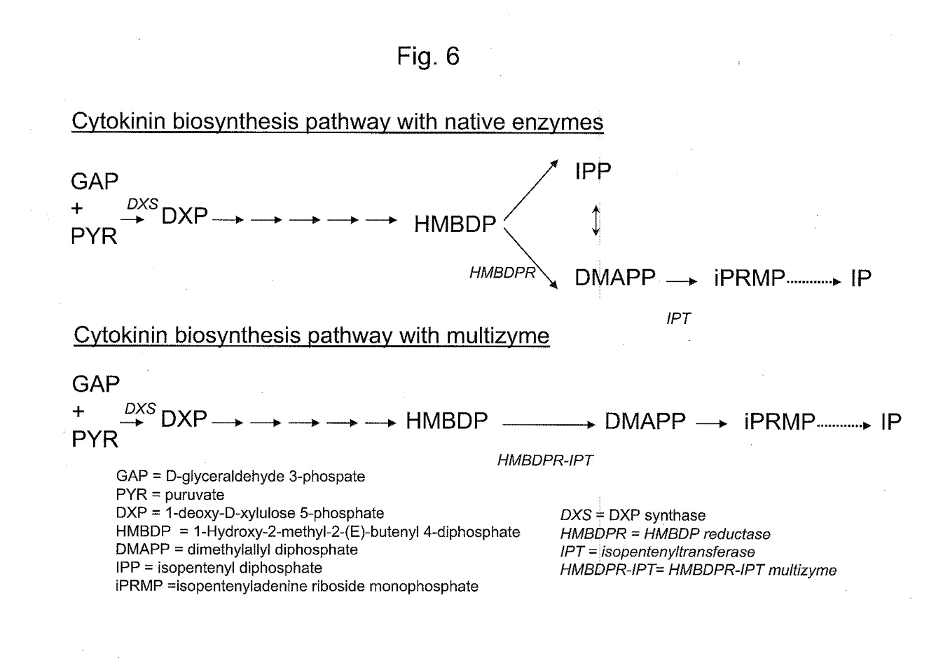

Isoprenoids are synthesized through the condensation of five-carbon intermediates, isopentenyl diphosphate (IPP) and dimethylallyl diphosphate (DMAPP), derived form the 2-C-methyl-D-erythritol 4-phosphate (MEP) pathway in plastids and the mevalonate (MVA) pathway in the cytosol. (FIG. 6) Kasahara et al 2004 (J. Biol. Chem. 2004; 279:14049-14054) demonstrated a critical contribution of the plastid-located MEP pathway in providing the prenyl precursor to tZ- and iP-type CKs in Arabidopsis seedlings. In the MEP pathway, 1-Hydroxy-2-methyl-2-(E)-butenyl 4-diphosphate (HMBDP) is reduced by HMBDP reductase (IDS; EC 1.17.1.2) to produce both IPP and DMAPP (Altincicek B, Kollas A, Eberl M, Wiesner J, Sanderbrand S, Hintz M, Beck E, Jomaa H (2001) LytB, a novel gene of the 2-C-methyl-D-erythritol 4-phosphate pathway of isoprenoid biosynthesis in Escherichia coli. FEBS Lett 499:37-40; Hecht S, Eisenreich W, Adam P, Amslinger S, Kis K, Bacher A, Arigoni D, Rohdich F (2001) Studies on the nonmevalonate pathway to terpenes: the role of the GcpE (IspG) protein. Proc Natl Acad Sci USA 98:14837-14842).

A fusion with HMBDP reductase and IPT can channel carbon into cytokinin by maximizing DMAPP production and reducing IPP formation (FIG. 6). Such a multizyme can also serve to divert more DMAPP into the CK pathway rather than to isoprenenoids. Furthermore, the use of cell-cycle specific promoters (as described in US2006/0010515A1) to drive the expression of the HMBDP-IPT fusion gene may result a yield increase

A HMBDP/IPT multizyme is created by joining an HMBDP reductase and IPT. IPT genes from several organisms have been identified to date (Takei, et al T. (2001) J. Biol. Chem. 276, 26405-26410, Kakimoto, (2001) Plant Cell Physiol. 42, 677-685; Sakamoto et al Plant Physiol 2006 Sep;142(1):54-62; Sugawara et al Proc Natl Acead Sci USA 2008 Feb. 19; 105(7):2734-9). Plant and microbial HMBDP reductase IPT genes have also been identified (Campos et al Biochem J 2001 Jan. 1; 353(Pt 1):59-67; Lu et al Mol Biol Rep 2007 2007 May 26 [Epub ahead of print]; Kim et al Planta 2008 January; 227(2):287-98)

The HMBDP reductase and IPT are joined together using a linker derived from a sequence of a DHA synthase locus of a Euglenoid. An example of such a linker is the EgDHAsyn1 proline-rich linker (SEQ ID NO:34). The gene fusion encoding the multizyme may be produced as described in Example 4, or as described by Examples 15-24 of Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety. Furthermore, any number of methods known to those skilled in the art may be used to produce the gene fusion. Additional nucleotides can be added to the 3′ end of the EgDHAsyn1 proline-rich linker sequence to enable cloning when making the fusions for all constructs. Thus, an additional 4 amino acids can be included between the end of the EgDHAsyn1 proline-rich linker (SEQ ID NO:34; PARPAGLPPATYYDSLAV) and the start of HMBDP reductase or the IPT used.

For comparison, constructs that co-expressed the independent and separate activities of the multizyme (HMBDP reductase and IPT) are also created. The multizymes may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the multiple enzymes.

The multizyme fusion gene can be expressed in either microbial cells as described in General Methods, monocot cells as in Example 5 or dicot cells as described in Example 2 and 3, or expression in eukaryotic cell culture, in planta, and using viral expression systems in suitably infected organisms or cell lines. Purification of the instant multizymes, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. The purification protocol may include the use of an affinity resin containing ligands which are specific for the independent and separate activities of the multizyme.

Crude, partially purified or purified enzyme may be utilized in assays for the evaluation of compounds for their ability to inhibit enzymatic activation of the instant multizymes disclosed herein. Assays may be conducted under well known experimental conditions which permit optimal enzymatic activity. For example, assays for HMBDP reductase (also referred to as lytB or IDS or isopentenyl diphosphate/dimethylalyl diphosphate synthase) are presented by Rohdich F, Hecht S, Gärtner K, Adam P, Krieger C, Amslinger S, Arigoni D, Bacher A, Eisenreich W (2002) Studies on the nonmevalonate terpene biosynthetic pathway: metabolic role of lspH (LytB) protein. Proc Natl Acad Sci USA 99: 1158-1163) and assays for IPT by by Kakimoto, T. (2001) Plant Cell Physiol. 42, 677-685 and Takei, et al. (2003) J. Plant Res. 116, 265-269.

Hence, this example describes the synthesis of multizymes having at least two independent and separable activities (HMBDP reductase and IPT) wherein the independent and separable activities are joined together using a linker derived from a sequence of a DHA synthase locus of a Euglenoid and wherein the product of a first reaction of the multizyme (DMAPP) is substantially used in a subsequent reaction (IPT) of the multizyme. In this manner, the producition of IPP can be reduced to a minimum due to the unique properties of the multizyme.

### Example 8

### Synthesis of Multizymes wherein at Least One Independent and Separable Activity of the Multizyme is Prevented from Associating with Polypeptide Complexes Other than the Multizyme of which it is a Part

This example describes the synthesis of a multizyme wherein at least one independent and separable activity of the multizyme is prevented from associating with polypeptide complexes other than the multizyme of which it is a part.

The availability of crops that have an efficient and effective resistance to herbicides continue to be of great importance to growers. Developing more effective resistance mechanism can allow for better weed management. A highly herbicide-resistant acetolactate synthase (HRA) has been described (Bedbrook J. R., Chaleff R. S., Falco S. C., Mazur B. J., Somerville C. R., and Yadev N. S. 1995). Nucleic acid fragment encoding herbicide resistant plant acetolactate synthase. U.S. Pat. No. 5,378,824.; Lee K. Y., Townsend J., Tepperman J., Black M., Chui C. F., Mazur B., Dunsmuir P., and Bedbrook J. 1988. The resistance mechanism to ALS-inhibiting herbicides is a double-mutant, highly resistant ALS (HRA) that is insensitive to all five classes of ALS herbicides (J. Green. 2006 Weed technology 21:547-558). Plants such as soybean produce a native acetolactate synthase (ALS) that can form a heterodimer with HRA in plant expressing the HRA monomer. In order to increase the herbicide resistance of HRA, it is desirable to prevent the formation of the HRA and ALS heterodimer and instead stimulate the formation of a HRA-HRA homodimer.

A HRA/HRA multizyme is made by joining an herbicide resistant acetolactate (HRA) synthase peptide to a second acetolactate synthase peptide. The acetolactate synthases are joined together using a linker derived from a sequence of a DHA synthase locus of a Euglenoid. An example of such a linker is the EgDHAsyn1 proline-rich linker (SEQ ID NO:34). The gene fusion encoding the multizyme may be produced as described in Example 4, described herein, or as described by Examples 15-24 of Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety. Furthermore, any number of methods known to those skilled in the art may be used to produce the gene fusion. Additional nucleotides can be added to the 3′ end of the EgDHAsyn1 proline-rich linker sequence to enable cloning when making the fusions for all constructs. Thus, an additional 4 amino acids can be included between the end of the EgDHAsyn1 proline-rich linker (SEQ ID NO:34; PARPAGLPPATYYDSLAV) and the start of the second HRA used. The HRA-EgDHAsyn1 proline-rich linker-HRA gene fusion encodes a homodimer that can prevent HRA peptides to form a heterodimer with a native acetolactase synthase (ALS).

The multizyme fusion gene can be expressed in either microbial cells as described in General Methods, monocot cells as described in Example 5 or dicot cells as described in Example 2 and 3, or expression in eukaryotic cell culture, in planta, and using viral expression systems in suitably infected organisms or cell lines. Purification of the instant multizymes, if desired, may utilize any number of separation technologies familiar to those skilled in the art of protein purification.

For comparison, a construct that expresses the monomer of HRA is also created using techniques well known in the art. Such a construct encodes a monomer of HRA that will form a heterodimer with the native ALS when expressed in a plant tissue that enables expression of native ALS.

Plants expressing the HRA-HRA homodimer versus the HRA-ALS heterodimer can be evaluated for altered herbicide resistance or altered agronomic properties. The multizyme can be evaluated by making transgenic soy plants and spraying them with sulfonyl ureas like rimsulfuron (Resolve) to see if they have improved tolerance using herbicide greenhouse tests well known to one skilled in the art.

### Example 9

### Synthesis of Multizymes for Increased Nitrogen Utilization

This example describes the synthesis of a multizyme wherein a product of an enzymatic reaction of the multizyme is channeled into a transporter activity which is comprised by the activity of the multizyme.

A NR/NT multizyme is created by joining a soluble nitrate reductase (NR) to a nitrate transporter (NT). The NR and the NT are joined together using a linker derived from a sequence of a DHA synthase locus of a Euglenoid. An example of such a linker is the EgDHAsyn1 proline-rich linker (SEQ ID NO:34). The gene fusion encoding the multizyme may be produced as described in Example 4, or as described by Examples 15-24 of Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety. Furthermore, any number of methods known to those skilled in the art may be used to produce the gene fusion. Additional nucleotides can be added to the 3′ end of the EgDHAsyn1 proline-rich linker sequence to enable cloning when making the fusions for all constructs. Thus, an additional 4 amino acids can be included between the end of the EgDHAsyn1 proline-rich linker (SEQ ID NO:34; PARPAGLPPATYYDSLAV) and the start of the NT used. The NR/NT multizyme can channel nitrogen through the transporter more effectively when compared to an individual and separate nitrate transported. Hence, the multizyme enables the product of the first protein (e. g. the nitrate reductase) to be channeled into a second protein which is a nitrate transporter resulting in an increased nitrogen uptake and utilization

The NR/NT fusion gene can be expressed in either microbial cells as described in General Methods, monocot cells as described in Example 5 or dicot cells as described in Example 2 and 3, or expression in eukaryotic cell culture, in planta, and using viral expression systems in suitably infected organisms or cell lines. Purification of the instant multizymes, if desired, may utilize any number of separation technologies familiar to those skilled in the art of protein purification.

For comparison, a construct that expresses the independent and separate activities of the multizyme (the NR and the nitrate transporter) are also created using techniques well known in the art. Plants expressing the NR/NT multizyme can be evaluated for altered nitrogen utilization using as described in, but not limited to, Example 5, or evaluated for altered agronomic traits using methods well known in the art.

### Example 10

### Transformation of Gaspe Flint Derived Maize Lines Containing the NR/NT Multizyme

Maize plants can be transformed as described in Example 5 to express the NR/NT gene fusion of Example 9. In addition to the promoters previously described other promoters such the S2B promoter, the maize ROOTMET2 promoter, the maize Cyclo, the CR1BIO, the CRWAQ81 and the maize ZRP2.4447 may be useful in expressing genes in maize. Furthermore, a variety of terminators, such as, but not limited to the PINII terminator, can be used to achieve expression of the gene of interest in Gaspe Flint Derived Maize Lines.

Recipient Plants

Recipient plant cells can be from a uniform maize line having a short life cycle (“fast cycling”), a reduced size, and high transformation potential. Typical of these plant cells for maize are plant cells from any of the publicly available Gaspe Flint (GBF) line varieties. One possible candidate plant line variety is the F1 hybrid of GBF×QTM (Quick Turnaround Maize, a publicly available form of Gaspe Flint selected for growth under greenhouse conditions) disclosed in Tomes et al. U.S. Patent Application Publication No. 2003/0221212. Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line is a double haploid line of GS3 (a highly transformable line)×Gaspe Flint. Yet another suitable line is a transformable elite inbred line carrying a transgene which causes early flowering, reduced stature, or both.

Transformation Protocol

Any suitable method may be used to introduce the transgenes into the maize cells, including but not limited to inoculation type procedures using Agrobacterium based vectors. Transformation may be performed on immature embryos of the recipient (target) plant.

Precision Growth and Plant Tracking

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks, and each plant is randomly assigned a location with the block.

For a group of thirty plants, twenty-four transformed, experimental plants and six control plants (plants with a set phenotype) (collectively, a “replicate group”) are placed in pots which are arranged in an array (a.k.a. a replicate group or block) on a table located inside a greenhouse. Each plant, control or experimental, is randomly assigned to a location with the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified and tracked throughout the evaluation process, and the data gathered from that plant is automatically associated with that plant so that the gathered data can be associated with the transgene carried by the plant. For example, each plant container can have a machine readable label (such as a Universal Product Code (UPC) bar code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor. See U.S. Published Patent Application No. 2004/0122592, incorporated herein by reference.

Phenotypic Analysis Using Three-Dimensional Imaging

Each greenhouse plant in the T0 event population, including any control plants, is analyzed for agronomic characteristics of interest, and the agronomic data for each plant is recorded or stored in a manner so that it is associated with the identifying data (see above) for that plant. Confirmation of a phenotype (gene effect) can be accomplished in the T1 generation with a similar experimental design to that described above.

The T0 plants are analyzed at the phenotypic level using quantitative, non-destructive imaging technology throughout the plant's entire greenhouse life cycle to assess the traits of interest. Preferably, a digital imaging analyzer is used for automatic multi-dimensional analyzing of total plants. The imaging may be done inside the greenhouse. Two camera systems, located at the top and side, and an apparatus to rotate the plant, are used to view and image plants from all sides. Images are acquired from the top, front and side of each plant. All three images together provide sufficient information to evaluate the biomass, size and morphology of each plant.

Due to the change in size of the plants from the time the first leaf appears from the soil to the time the plants are at the end of their development, the early stages of plant development are best documented with a higher magnification from the top. This may be accomplished by using a motorized zoom lens system that is fully controlled by the imaging software.

In a single imaging analysis operation, the following events occur: (1) the plant is conveyed inside the analyzer area, rotated 360 degrees so its machine readable label can be read, and left at rest until its leaves stop moving; (2) the side image is taken and entered into a database; (3) the plant is rotated 90 degrees and again left at rest until its leaves stop moving, and (4) the plant is transported out of the analyzer.

Plants are allowed at least six hours of darkness per twenty four hour period in order to have a normal day/night cycle.

Imaging Instrumentation

Any suitable imaging instrumentation may be used, including but not limited to light spectrum digital imaging instrumentation commercially available from LemnaTec GmbH of Wurselen, Germany. The images are taken and analyzed with a LemnaTec Scanalyzer HTS LT-0001-2 having a ½″ IT Progressive Scan IEE CCD imaging device. The imaging cameras may be equipped with a motor zoom, motor aperture and motor focus. All camera settings may be made using LemnaTec software. Preferably, the instrumental variance of the imaging analyzer is less than about 5% for major components and less than about 10% for minor components.

Software

The imaging analysis system comprises a LemnaTec HTS Bonit software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems (e.g. Matlab, others) can be used for quantitative interpretation of the imaging data, and any of these software systems can be applied to the image data set.

Conveyor System

A conveyor system with a plant rotating device may be used to transport the plants to the imaging area and rotate them during imaging. For example, up to four plants, each with a maximum height of 1.5 m, are loaded onto cars that travel over the circulating conveyor system and through the imaging measurement area. In this case the total footprint of the unit (imaging analyzer and conveyor loop) is about 5 m×5 m.

The conveyor system can be enlarged to accommodate more plants at a time. The plants are transported along the conveyor loop to the imaging area and are analyzed for up to 50 seconds per plant. Three views of the plant are taken. The conveyor system, as well as the imaging equipment, should be capable of being used in greenhouse environmental conditions.

Illumination

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternaively, the illumination can be varied to cause excitation of either transgene (e.g., green fluorescent protein (GFP), red fluorescent protein (RFP)) or endogenous (e.g. Chlorophyll) fluorophores.

Biomass Estimation Based on Three-Dimensional Imaging

For best estimation of biomass the plant images should be taken from at least three axes, preferably the top and two side (sides 1 and 2) views. These images are then analyzed to separate the plant from the background, pot and pollen control bag (if applicable). The volume of the plant can be estimated by the calculation:

$$Volume ( voxels ) = \sqrt{TopArea ( pixels )} \times \sqrt{Side 1 Area ( pixels )} \times \sqrt{Side 2 Area ( pixels )}$$

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification

The imaging technology may also be used to determine plant color and to assign plant colors to various color classes. The assignment of image colors to color classes is an inherent feature of the LemnaTec software. With other image analysis software systems color classification may be determined by a variety of computational approaches.

For the determination of plant size and growth parameters, a useful classification scheme is to define a simple color scheme including two or three shades of green and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time, or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins), and that other color classification schemes can provide further measures of gene action in traits related to these responses.

Plant Architecture Analysis

Transgenes which modify plant architecture parameters may also be identified using the present invention, including such parameters as maximum height and width, internodal distances, angle between leaves and stem, number of leaves starting at nodes and leaf length. The LemnaTec system software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g. pollination date, first silk date) can be recorded by the personnel responsible for performing plant care. To maximize data integrity and process efficiency this data is tracked by utilizing the same barcodes utilized by the LemnaTec light spectrum digital analyzing device. A computer with a barcode reader, a palm device, or a notebook PC may be used for ease of data capture recording time of observation, plant identifier, and the operator who captured the data.

Orientation of the Plants

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant, and an additional rotating device is used to turn the plant to the appropriate orientation prior to starting the main image acquisition.

### Example 11

### Screening of Transgenic Maize Lines

### Expressing the NR/NT Multizyme Under Nitrogen Limiting Conditions

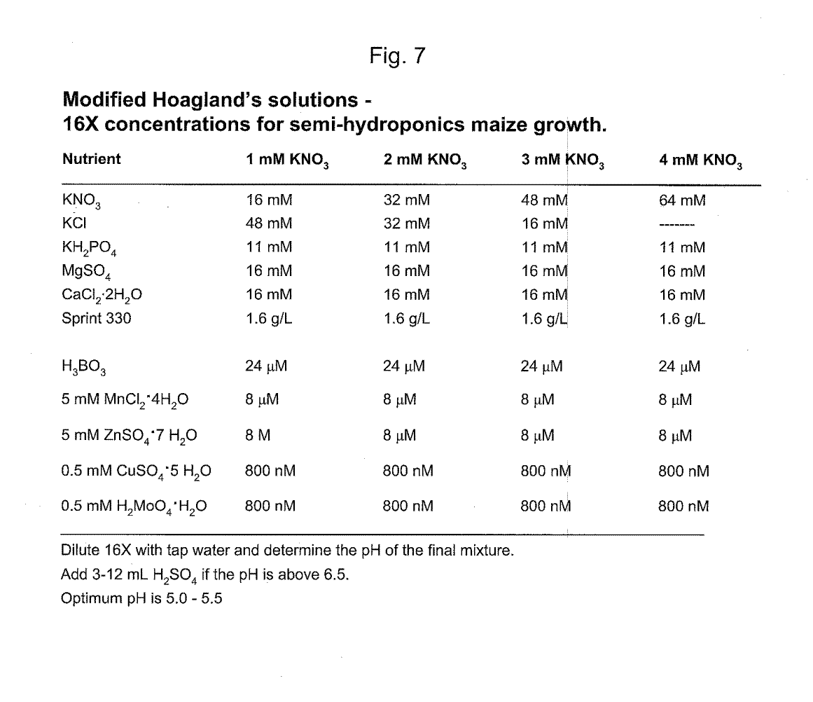

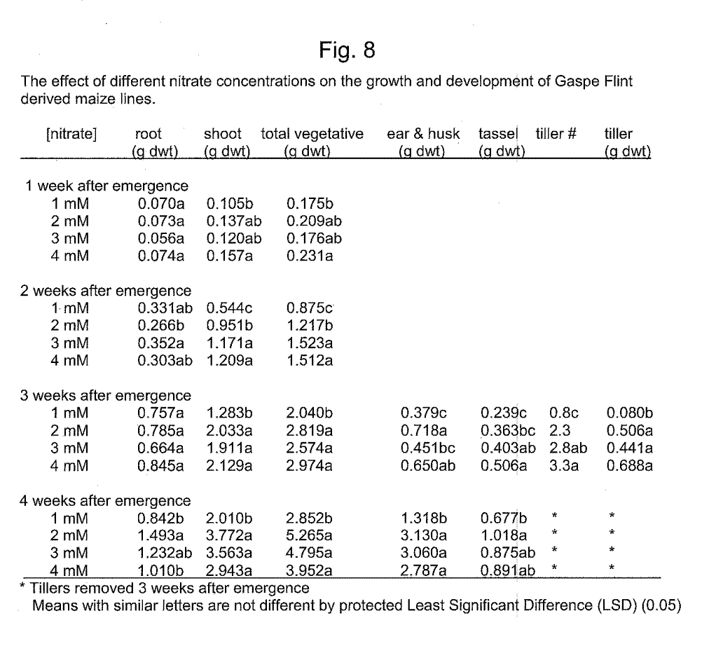

Transgenic plants containing the NR/NT multizyme are planted in Turface, a commercial potting medium, and watered four times each day with 1 mM KNO₃ growth medium and with 2 mM KNO₃, or higher, growth medium (see FIG. 7). Control plants grown in 1 mM KNO₃ medium will be less green, produce less biomass and have a smaller ear at anthesis (see FIG. 8 for an illustration of sample data).

Statistics are used to decide if differences seen between treatments are really different. FIG. 8 illustrates one method which places letters after the values. Those values in the same column that have the same letter (not group of letters) following them are not significantly different. Using this method, if there are no letters following the values in a column, then there are no significant differences between any of the values in that column or, in other words, all the values in that column are equal.

Expression of a transgene will result in plants with improved plant growth in 1 mM KNO₃ when compared to a transgenic null. Thus biomass and greenness (as described in Example 10) will be monitored during growth and compared to a transgenic null. Improvements in growth, greenness and ear size at anthesis will be indications of increased nitrogen tolerance.

### Example 12

### Creation of Multizymes for High Stearic-Low Palmitic Oilseed Plants

This example describes the creation of a multizyme wherein at least one activity of the multizyme that is capable of multiple functions when expressed as an individual polypeptide is restricted to a single function when it is part of the multizyme.

A multizyme is created by joining a type B acyl-ACP thioesterase (16:0/18:0 specific) with a beta-ketoacyl-ACP synthetase II protein (KAS II). The type B acyl-ACP thioesterase and KAS II are joined together using a linker derived from a sequence of a DHA synthase locus of a Euglenoid. An example of such a linker is the EgDHAsyn1 proline-rich linker (SEQ ID NO:34). The gene fusion encoding the multizyme may be produced as described in Example 4, or as described by Examples 15-24 of Applicants' Assignee's application having U.S. application Ser. No. 12/061,738 (filed Apr. 3, 2008, which published Oct. 16, 2008; Attorney Docket No. BB-1585 USNA), the subject matter of which is hereby incorporated by reference in its entirety. Furthermore, any number of methods known to those skilled in the art may be used to produce the gene fusion. Additional nucleotides can be added to the 3′ end of the EgDHAsyn1 proline-rich linker sequence to enable cloning when making the fusions for all constructs. Thus, an additional 4 amino acids can be included between the end of the EgDHAsyn1 proline-rich linker (SEQ ID NO:34; PARPAGLPPATYYDSLAV) and the start of the KASII used.

For comparison, constructs that co-expressed the independent and separate activities of the multizyme (type B acyl-ACP thioesterase and KASII) are also created. The multizymes may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the multiple enzymes.

The multizyme fusion gene can be expressed in either microbial cells as described in General Methods, monocot cells as described in Example 5 or dicot cells as described in Example 2 and 3, or expression in eukaryotic cell culture, in planta, and using viral expression systems in suitably infected organisms or cell lines. Purification of the instant multizymes, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor.

Crude, partially purified or purified enzyme may be utilized in assays for the evaluation of compounds for their ability to inhibit enzymatic activation of the instant multizymes disclosed herein. Assays may be conducted under well known experimental conditions which permit optimal enzymatic activity. For example, an assay for KAS II (beta-ketoacyl-ACP synthetase) is described in U.S. Pat. No. 5,500,361 issued on Mar. 19, 1996 and assays for thioesterase is described in US Reissued Patent RE37,317, reissued on Aug. 7, 2001 Plants expressing acyl-ACP thioesterase/KASII multizyme can be evaluated for their oil quality composition. The expression of an acyl-ACP thioesterase/KASII multizyme in an oilseed plastid can result in the channeling of 18:0-ACP in to the thioesterase so that it releases only 18:0 and not 16:0 during plastidal acyl-ACP synthesis (and also prevent the 18:0 being desaturated). This may lead to the creation of an oilseed containing a high stearic, low palmitic oil.

What is claimed is:

1. A multizyme comprising a single polypeptide having at least two independent and separable activities wherein the independent and separable activities are joined together using a linker derived from a sequence of a DHA synthase locus of a Euglenoid wherein said linker can be natural or synthetic provided that the multizyme does not comprise (i) a fatty acid desaturase linked to a fatty acid elongase or (ii) a fatty acid desaturase linked to another fatty acid desaturase.

2. The multizyme of claim 1 wherein use of the multizyme increases total flux of all combined reactions of the multizyme when compared with the total flux of the combined reactions when each reaction is produced separately when not linked together as the multizyme.

3. The multizyme of claim 1 wherein use of the multizyme in a reaction sequence lowers an amount of at least one intermediate produced by the multizyme when compared to the amount of substantially the same intermediate produced by each individual enzymatic activity in a reaction sequence when not linked together as a multizyme.

4. The multizyme of claim 1 wherein use of the multizyme in a reaction reduces a extent ofany feedback inhibition of at least one enzymatic activity of the multizyme when compared to the amount of feedback inhibition of a corresponding enzymatic activity that occurs when said individual enzymatic activity is not part of a multizyme.

5. The multizyme of claim 1 wherein use of the multizyme in a reaction protects an activity of the multizyme from competitive inhibition by a substrate other than that produced by a preceding enzymatic activity of the multizyme.

6. The multizyme of claim 1 wherein a product of a reaction of the multizyme is substantially used in a subsequent reaction of the multizyme.

7. The multizyme of claim 1 wherein at least one independent and separable activity of the multizyme is prevented from associating with polypeptide complexes other than the multizyme of which it is a part.

8. The multizyme of claim 1 wherein a product of an enzymatic reaction of the multizyme is channeled into a transporter activity which is comprised by the of the multizyme.

9. The multizyme of claim 1 wherein at least one activity of the multizyme that is capable of multiple functions when expressed as an individual polypeptide is restricted to a single function when it is part of the multizyme.

10. The multizyme of claim 5 wherein said multizyme comprisesat least two enzymatic activities which when linked together have resistance to an herbicide inhibitor of at least one of the individual activities

11. The multizyme of claim 5 wherein said multizyme comprises a 4-hydroxyphenyl pyruvate dioxygenase linked to an activity that produces hydroxyphenylpyruvate.

12. The multizyme of claim 5 wherein said multizyme comprises a 4-hydroxyphenyl pyruvate dioxygenase linked to a prephenate dehydrogenase or a bifunctional chorismate mutase.

13. The multizyme of claim 6 wherein expression of said multizyme in a transgenic plant confers an increase in seed yield or drought resistance of said plant.

14. The multizyme of claim 6 wherein said multizyme comprises a 1-hydroxy-2-methyl-2-(E)-butenyl 4-diphosphate reductase linked to adenosine phosphate-isopentenyltransferase.

15. The multizyme of claim 7 wherein said multizyme comprises an herbicide resistant acetolactate synthase peptide linked to a second herbicide resistant acetolactate synthase peptide.

16. The multizyme of claim 8 wherein said multizyme comprises a soluble nitrate reductase linked to a nitrate transporter peptide.

17. The multizyme of claim 9 wherein said multizyme comprises an acyl-ACP-thioesterase protein linked to a beta-ketoacyl-ACP synthetase II protein.
